(19) United States
(12) Patent Application Publication
(10) Pub. No.: US 2026/0232581 A1
(43) Pub. Date: Aug. 13, 2026

(54) RESPIRATORY DRY POWDER DELIVERY

(71) Applicant: BOARD OF REGENTS, THE UNIVERSITY OF TEXAS SYSTEM, Austin, TX (US)

(72) Inventors: Zhengrong CUI, Austin, TX (US); Yu-Sheng YU, Austin, TX (US); Khaled ABOULFOTOUH, Austin, TX (US); Robert O. WILLIAMS, III, Austin, TX (US)

(21) Appl. No.: 19/116,236

(22) PCT Filed: Sep. 28, 2023
(86) PCT No.: PCT/US2023/075402

§ 371 Date: Mar. 27, 2025

Related U.S. Application Data
(60) Provisional application No. 63/377,384, filed on Sep. 28, 2022.

(51) Int. Cl. A61K 9/00; A61K 9/50; A61K 31/7105; A61K 39/00; A61K 39/112

(52) U.S. Cl. CPC A61K 9/0073; A61K 9/5068; A61K 31/7105; A61K 39/0275; A61K 2039/543; A61K 2039/55555; A61K 2039/55583

(57) ABSTRACT

The present disclosure provides pharmaceutical compositions for use in delivering therapeutics, such as vaccines or biologic material, to the nasal cavity or the deep lungs. In particular, the pharmaceutical compositions may preferentially deposit on the lower or middle turbinate region and the nasopharynx region of the nasal cavity. In other aspects the pharmaceutical compositions may be used to generate powders that penetrate the deep lungs. Also, provided herein are methods of preferential delivery to these regions.

The present application claims the benefit of priority to U.S. Provisional Application No. 63/377,384, filed on Sep. 28, 2022, the entire contents of which are hereby incorporated by reference.

### BACKGROUND

### I. Field

The disclosure generally relates to delivery of dry powders to the nose or to the lungs. More particularly, the disclosure relates to dry thin film compositions and pharmaceutical formulations that may include either polypeptides, polynucleotides, or lipids.

### II. Related Art

Data from previous studies have demonstrated that delivering vaccines by the intranasal route could induce systematic and mucosal immune responses (Birkhoff et al., 2009). Several nasal vaccines have been approved for human use around the world, including the Flumist Quadrivalent in the US and a nasal COVID-19 vaccine recently in India. Those nasal vaccines are virus-based and are presented as a liquid suspension or in a freeze-dried powder for reconstitution. The liquid vaccine is then administered intranasally using an intranasal sprayer. Intranasal administration of vaccines directly in a dry powder form has advantages, including storage and distribution, resident time in the nasal cavity, and the dose of the vaccine that can be administered.

Many viral infections, such as seasonal flu, respiratory syncytial virus (RSV) infection, the coronavirus disease 2019 (COVID-19), initiate in the upper respiratory tract, including the nasal cavity (Jain et al., 2022; Petersen et al., 2020; Pormohammad et al., 2021; van Riel et al., 2010). Monoclonal antibodies (mAbs) that neutralize the viruses can be used to treat infections. For example, several mAb products have been authorized by regulatory agencies for COVID-19 treatment or pre-exposure prevention, including GlaxoSmithKline and Vir Biotechnology's Sotrovimab (Heo, 2022), and AstraZeneca's Tixagevimab-Cilgavimab (Keam, 2022). These mAbs are administered by needle-based intravenous (IV) infusion or intramuscular (IM) injection (Kelley et al., 2022). Intranasal administration of the mAbs, alone or in combination with systemic injection of them, may improve patient outcomes, and data from pre-clinical and clinical studies support the feasibility of delivering such mAb products intranasally to neutralize SARS-CoV-2 in a mouse model or to reduce lung inflammation and blood inflammatory biomarkers in mild to moderate COVID-19 patients (Halwe et al., 2021; Moreira et al., 2021). However, intranasal delivery of mAbs in liquid formulations has drawbacks such as the limited volume that can be administered (Li et al., 2000) and the short residence time of the liquid in the nasal cavity (Filipović-Grčić & Hafner). Intranasal administration of mAbs as dry powders may have advantages, including a prolonged residence time in the nasal cavity, benefits in storage and distribution, and the ability to modify the dissolution of the mAbs from the dry powders (Djupesland, 2013; Filipović-&Grčić & Hafner; Nižić Nodilo et al., 2021).

Unfortunately, converting mAbs from liquid formulations to aerosolizable dry powders for intranasal delivery can be challenging. Conventional (shelf) freeze-drying of mAbs in the presence of various lyoprotectants such as disaccharides has proven feasible (Cleland et al., 2001; Haeuser et al., 2020), but shelf freeze-dried products are generally not suitable for aerosolization. Spray drying and spray freeze-drying may produce dry powders with desirable aerosol properties, but they are inherently associated with shear stress and high air-liquid interfacial surface area during the atomization process and the heat stress unique to spray drying (Emami et al., 2018), which can potentially damage the mAbs (Pabari et al., 2011). Thin-film freeze-drying is a technology that can be applied to engineer aerosolizable dry powders while avoiding shear and heat stresses (Zhang et al., 2021). Compared to conventional shelf freeze-drying, thin-film freeze-drying provides higher freezing rate (i.e., 100-1000 K/s vs. 0.5-1 K/min) and potentially higher drying rate (Engstrom et al., 2008; Wang et al., 2023). Importantly, the highly porous, brittle matrix structures and the low bulk density of the thin-film freeze-dried powders often have desirable aerosol properties (Praphawatvet et al., 2022). Previously, it has been demonstrated that it is feasible to apply thin-film freeze-drying to prepare dry powders of mAbs for pulmonary delivery by oral inhalation (Emig et al., 2021; Hufnagel et al., 2022; Sahakijpijarn et al., 2020). For example, AUG-3387, a human-derived mAb that neutralizes SARS-CoV-2, was successfully thin-film freeze-dried into aerosolizable dry powders for pulmonary delivery (Emig et al., 2021). However, a method to transform the mAbs from liquid to dry powders suitable for intranasal administration is needed to fully realize the potentials of the mAbs.

Synthetic oligodeoxynucleotides (ODNs) that contain unmethylated cytosine-phosphate-guanine (CpG) dinucleotide motifs possess potent immunostimulatory effects (Zhang & Gao, 2017). CpG ODNs can activate innate and adaptive immune responses through activation of Toll-like receptor (TLR) 9 (Perry et al., 2020). Binding of CpG ODNs to TLR9 receptors initiates a cascade of innate and adaptive immune responses that eventually result in the secretion of proinflammatory cytokines and chemokines, activation of natural killer cells and expansion of T cell population (Perry et al., 2020). Therefore, CpG ODNs hold a great promise as vaccine adjuvants and anticancer agents. In fact, CpG 1018 ODN is an adjuvant in HEPLISAV-B®, an FDA-approved hepatitis B vaccine. Additionally, CpG ODNs have been evaluated in clinical trials for controlling viral infections such as severe acute respiratory syndrome coronavirus 2 (e.g., NCT04962893 and NCT04818281). However, the immunotherapeutic application of CpG ODNs is limited by their poor cellular penetration and the degradation of natural CpG ODNs by nucleases (Perry et al., 2020). Local delivery of CpG ODNs or CpG ODN-containing vaccines to the nasal cavity or into the lungs can potentially alleviate the systemic side effects of CpG ODNs, and pulmonary as well as intranasally administered vaccines can potentially induced both systemic and mucosal specific immune responses.

Messenger RNA (mRNA) is an evolving class of nucleic acid-based therapeutics. The mRNA therapeutics have several advantages. For instance, mRNA is translated to proteins in the cytoplasm without the need for it to penetrate the nucleus or integrate into the host genome (Weng et al., 2020), and it can be synthesized by in vitro transcription (Schoenmaker et al., 2021). Therefore, mRNA is very promising candidate for gene therapy, cancer therapy and prevention of infectious diseases. Cellular internalization and gene expression are essential requirements for effective mRNA therapies. Lipid nanoparticles (LNPs) are the leading delivery platform for facilitating the cellular delivery of the large (i.e., 300-5,000 kDa), negatively charged, and chemically unstable mRNA (Weng et al., 2020). Spikevax™ and Comirnaty™ are the first in class FDA-approved mRNA therapeutics for the prevention of SARS-CoV-2 infection. Both vaccines are administered intramuscularly. For mRNA vaccines designed to prevent respiratory infections, there is an interest in eliciting protective mucosal immune responses in the respiratory tract (Kim & Jang, 2017). Intranasally administered vaccines are known to have the potential to induce specific immune responses not only in the systemic circulation, but also in the mucosal secretions of the respiratory tract (e.g., nasal mucosal secretion and lung mucosal secretion) (Cahn et al., 2023; Igyarto et al., 2021). Vaccines based on mRNA-LNPs may be administered intranasally as a liquid but converting these vaccines from a liquid suspension to aerosolizable dry powders for intranasal or pulmonary administration has advantages (e.g., avoid the need for extra cold freezing temperatures for storage, control the dissolution or release of the mRNA-LNPs after dosing).

Unfortunately, dry powders with the proper aerosol properties for desired deposition in the respiratory tract (i.e., nasal cavity and/or lungs) and a method to prepare the dry powders remain needed.

### SUMMARY

In an aspect there is provided methods of delivering a pharmaceutical composition to either the deep lung tissue or alternatively, to the lower or middle turbinate and the nasopharynx regions of the nose. These methods may be employed by using a powder that is prepared using a method that comprises a low concentration, such as less than 3% solids in feedstock, of the pharmaceutical composition in the feedstock to prepare a composition that may be used for deep lung delivery. Alternative, if a high concentration, such as greater than 3% solids in the feedstock, is used, then the composition may be useful for delivery intranasally. These compositions may be formulated for intranasal delivery and into a device capable of such delivery.

In some aspects, the present disclosure provides pharmaceutical compositions comprising:
(A) a therapeutic composition comprising:
   (i) an active pharmaceutical ingredient;
   (ii) an excipient, wherein the excipient is selected from a sugar, sugar alcohol, or an amino acid;
   (iii) a mucoadhesive excipient, and
(B) a nasal delivery device, wherein the therapeutic composition is loaded in the nasal device or formulated into a container that can be attached to the nasal delivery device; and the nasal delivery device produces an initial velocity upon delivering the therapeutic composition of at least 200 cm/s.

In some embodiments, the active pharmaceutical ingredient is a vaccine formulation. In some embodiments, the active pharmaceutical ingredient is formulated as a liposome or comprises a liposome. In some embodiments, the active pharmaceutical ingredient further comprises an adjuvant. In some embodiments, the adjuvant is an inorganic adjuvant. In other embodiments, the adjuvant is an organic adjuvant. In some embodiments, the adjuvant further comprises two adjuvants. In some embodiments, the adjuvant comprises a first adjuvant selected from a lipid. In some embodiments, the first adjuvant is Lipid A. In some embodiments, the adjuvant comprises a second adjuvant selected from a natural product. In some embodiments, the second adjuvant is a saponin. In some embodiments, the second adjuvant is QS-21.

In some embodiments, the active pharmaceutical ingredient is an antigen. In some embodiments, the antigen is an antigen for an infection. In some embodiments, the infection is a viral infection or a bacterial infection. In some embodiments, the infection is a viral infection. In other embodiments, the infection is a bacterial infection. In some embodiments, the antigen is a protein antigen. In other embodiments, the antigen is an attenuated antigen. In other embodiments, the antigen is an inactivated antigen. In other embodiments, the antigen is a subunit antigen. In other embodiments, the antigen is a virus-like particle.

In some embodiments, the active pharmaceutical ingredient is formulated into or admixed a liposome with at least one lipid and one sterol. In some embodiments, the liposome comprises a phospholipid such as DOPC. In some embodiments, the lipid comprises cholesterol.

In some embodiments, the excipient is a sugar. In some embodiments, the sugar is disaccharide. In some embodiments, the sugar is sucrose, lactose, maltose, or trehalose. In some embodiments, the sugar is sucrose.

In some embodiments, the mucoadhesive polymer is a cellulosic polymer. In some embodiments, the mucoadhesive polymer is a charged cellulosic polymer. In some embodiments, the charged cellulosic polymer is a negatively charged cellulosic polymer. In some embodiments, the charged cellulosic polymer is carboxymethyl cellulose. In some embodiments, the mucoadhesive excipient comprises an amount from about 0.1% to about 16% w/w of the composition relative to the total weight of the components. In some embodiments, the amount of mucoadhesive excipient is from about 0.5% to about 12.5% w/w. In some embodiments, the amount of the mucoadhesive excipient is from about 1% to about 10% w/w.

In some embodiments, the active pharmaceutical ingredient comprises an amount from about 10 μg to about 1 mg. In some embodiments, the amount of the active pharmaceutical ingredient is from about 25 μg to about 500 μg. In some embodiments, the amount of the active pharmaceutical ingredient is from about 40 μg to about 100 μg.

In some embodiments, the pharmaceutical composition further comprises an adjuvant in an amount from about 10 μg to about 1 mg. In some embodiments, the amount of the active pharmaceutical ingredient is from about 25 μg to about 500 μg. In some embodiments, the amount of the active pharmaceutical ingredient is from about 40 μg to about 100 μg.

In some embodiments, the pharmaceutical composition comprises a first and second adjuvant with a ratio of the first and second adjuvant of 5:1 to about 1:5. In some embodiments, the ratio is from about 2:1 to about 1:2. In some embodiments, the ratio is about 1:1.

In some embodiments, the pharmaceutical compositions further comprise a further excipient. In some embodiments, the further excipient is a buffer. In some embodiments, the further excipient is a phosphate buffer. In some embodiments, the further excipient is a salt. In some embodiments, the salt is sodium chloride. In some embodiments, the further excipient is phosphate buffered saline.

In some embodiments, the pharmaceutical composition comprises one or more drug particles in which each of the particles comprise the active pharmaceutical ingredient, the mucoadhesive excipient, and the excipient. In some embodiments, the drug particles have an average particle size from about 25 nm to about 250 nm. In some embodiments, the average particle size is from about 50 nm to about 200 nm. In some embodiments, the average particle size is from about 75 nm to about 150 nm. In some embodiments, the active pharmaceutical ingredient, the mucoadhesive excipient, or an excipient are in the amorphous form. In some embodiments, the pharmaceutical composition comprises a further excipient in the crystalline form.

In some embodiments, the initial velocity is greater than 250 cm/s. In some embodiments, the initial velocity is greater than 300 cm/s. In some embodiments, the initial velocity is greater than 400 cm/s.

In some aspects, the present disclosure provides methods of treating a disease or disorder in a patient comprising administering to the nasal cavity of the patient a therapeutically effective amount of a pharmaceutical composition described herein.

In other aspects, the present disclosure provides methods of preventing a disease or disorder in a patient comprising administering to the naval cavity of the patient a therapeutically effective amount of a pharmaceutical composition described herein.

In some aspects, the present disclosure provides compositions for use in the preparation of a medicament for the treatment of a disease or disorder in a patient comprising a therapeutically effective amount of a pharmaceutical composition described herein.

In other aspects, the present disclosure provides compositions for use in the preparation of a medicament for the prevention of a disease or disorder in a patient comprising a therapeutically effective amount of a pharmaceutical composition described herein.

In some aspects, the present disclosure provides uses of a pharmaceutical composition described herein in the preparation of a medicament for the treatment of a disease or disorder.

In other aspects, the present disclosure provides uses of a pharmaceutical composition described herein in the preparation of a medicament for the prevention of a disease or disorder.

In some embodiments, the disease or disorder is an infection. In some embodiments, the disease or disorder is an infection of a bacteria. In other embodiments, the disease or disorder is an infection of a virus.

In still yet another aspect, the present disclosure provides methods of preferentially delivering an active pharmaceutical ingredient to either the lower turbinate, middle turbinate, or the nasopharynx region of the nasal cavity comprising administering to the nasal cavity of the person a pharmaceutical composition described herein, wherein at least 45% of the active pharmaceutical ingredient is delivered to the lower turbinate, middle turbinate, or the nasopharynx region.

In some embodiments, at least 50% of the active pharmaceutical ingredient is delivered to the lower turbinate, middle turbinate, or the nasopharynx region. In some embodiments, at least 60% of the active pharmaceutical ingredient is delivered to the lower turbinate, middle turbinate, or the nasopharynx region. In some embodiments, at least 70% of the active pharmaceutical ingredient is delivered to the lower turbinate, middle turbinate, or the nasopharynx region.

In some embodiments, at least 20% of the active pharmaceutical ingredient is delivered to the lower turbinate region. In some embodiments, at least 25% of the active pharmaceutical ingredient is delivered to the lower turbinate region. In some embodiments, at least 30% of the active pharmaceutical ingredient is delivered to the lower turbinate region. In some embodiments, at least 15% of the active pharmaceutical ingredient is delivered to the middle turbinate region. In some embodiments, at least 20% of the active pharmaceutical ingredient is delivered to the middle turbinate region.

In some embodiments, at least 2.5% of the active pharmaceutical ingredient is delivered to the nasopharynx region. In some embodiments, at least 5% of the active pharmaceutical ingredient is delivered to the nasopharynx region. In some embodiments, at least 7.5% of the active pharmaceutical ingredient is delivered to the nasopharynx region.

In some embodiments, the methods result in less than 15% of the active pharmaceutical ingredient in the upper turbinate region. In some embodiments, the methods result in less than 10% of the active pharmaceutical ingredient in the upper turbinate region. In some embodiments, the methods result in less than 7.5% of the active pharmaceutical ingredient in the upper turbinate region. In some embodiments, the methods result in less than 30% of the active pharmaceutical ingredient in the anterior region. In some embodiments, the methods result in less than 25% of the active pharmaceutical ingredient in the anterior region. In some embodiments, the methods result in less than 20% of the active pharmaceutical ingredient in the anterior region.

In still another aspect, the present disclosure provides methods of delivering a biologic active agent to the nasal cavity of a patient comprising:
(A) obtaining a pharmaceutical composition comprising:
   (1) a biologic active agent;
   (II) a sugar or sugar alcohol; and
   (III) a buffer;
   wherein the pharmaceutical composition comprises a plurality of drug particles having an average diameter from about 50 μm to about 1 cm;
(B) delivering the biologic active agent to the nasal cavity by ejecting the powder from a device into the nostril.

In some embodiments, the biologic active agent is an antibody or a fragment of an antibody. In some embodiments, the biologic active agent is an antibody such as a monoclonal antibody. In some embodiments, the antibody is an antibody against a virus. In some embodiments, the virus is a respiratory virus. In some embodiments, the virus is a coronavirus. In some embodiments, the biologic active agent is an antibody against the SARS-CoV2 virus. In other embodiments, the biologic active agent is a protein. In other embodiments, the biologic active agent is a peptide.

In some embodiments, the pharmaceutical composition comprises a sugar alcohol. In some embodiments, the sugar alcohol is a 5 or 6 carbon sugar alcohol. In some embodiments, the sugar alcohol is mannitol. In some embodiments, the pharmaceutical compositions comprise a weight ratio of the sugar or sugar alcohol to the biologic active agent is greater than 5:1. In some embodiments, the weight ratio is greater than 10:1. In some embodiments, the buffer is a buffer with a pH range around 6-8. In some embodiments, the buffer is Tris, phosphate, or histidine. In some embodiments, the buffer is histidine.

In some embodiments, the pharmaceutical compositions further comprise one or more excipients. In some embodiments, the excipient is an amino acid. In some embodiments, the amino acid is a non-ionizable amino acid. In some embodiments, the excipient is leucine.

In some embodiments, the pharmaceutical compositions further comprise a surfactant. In some embodiments, the surfactant is a non-ionic surfactant. In some embodiments, the surfactant is a PEGylated sorbitol derivative. In some embodiments, the PEGylated sorbitol derivative further comprises a fatty acid tail. In some embodiments, the fatty acid tail is a fatty acid with from 12 to 18 carbon atoms. In some embodiments, the fatty acid tail is lauric acid. In some embodiments, the PEGylated sorbitol derivative comprises a polyethylene oxide group with a combined number of repeating units from about 10 to about 100. In some embodiments, the combined number of repeating units is from about 10 to about 30. In some embodiments, the combined number of repeating units is about 20.

In some embodiments, the pharmaceutical compositions comprise:
(A) a biologic active agent, wherein the biologic active agent is an antibody or antibody fragment;
(B) a sugar or sugar alcohol; wherein the ratio of the sugar or sugar alcohol to the biologic active agent is greater than 5:1;
(C) a buffer, wherein the buffer has a pH range is from about pH 6 to 8;
(D) an excipient, wherein the excipient is an amino acid; and
(E) a surfactant, wherein the surfactant is a non-ionic surfactant.

In some embodiments, the composition further comprises a polynucleotide. In some embodiments, the polynucleotide is a therapeutic polynucleotide. In some embodiments, the therapeutic polynucleotide is a deoxynucleotide. In some embodiments, the polynucleotide is a dinucleotide. In other embodiments, the polynucleotide is an oligonucleotide. In some embodiments, the polynucleotide is a class A oligonucleotide. In other embodiments, the polynucleotide is a class B oligonucleotide. In other embodiments, the polynucleotide is a class C oligonucleotide. In some embodiments, the oligonucleotide contain unmethylated cytosine-phosphate-guanine (CpG) motifs.

In some embodiments, the sugar or sugar alcohol is a sugar. In some embodiments, the sugar is trehalose, mannitol, lactose, sucrose, or maltose. In some embodiments, the sugar is lactose.

In some embodiments, the pharmaceutical compositions further comprise an excipient. In some embodiments, the excipient is an amino acid. In some embodiments, the amino acid is a non-ionizable amino acid. In some embodiments, the amino acid is leucine.

In some embodiments, the buffer is a buffer with a pH range around 6-9. In some embodiments, the buffer is Tris, phosphate, or histidine. In some embodiments, the buffer is Tris. In some embodiments, the buffer further comprises a chelating compound. In some embodiments, the chelating compound is polycarboxylic acid such as EDTA.

In some embodiments, the pharmaceutical compositions further comprise a second therapeutic agent. In some embodiments, the second therapeutic agent is an antigen. In some embodiments, the weight ratio of the sugar or sugar alcohol and the excipient is from about 5:1 to about 1:3. In some embodiments, the weight ratio is from about 3:1 to about 1:1. In some embodiments, the weight ratio is about 3:2. In some embodiments, the weight ratio of the sugar or sugar alcohol and the polynucleotide is from about 40:1 to about 250:1.

In some embodiments, the biologic active agent is a polynucleotide. In some embodiments, the polynucleotide is encapsulated in a lipid nanoparticle. In some embodiments, the polynucleotide is a therapeutic nucleotide. In some embodiments, the polynucleotide is a siRNA, a mRNA, an anti-sense oligonucleotide, a DNA or RNA aptamer, RNAi, miRNA, tRNA, or circular RNA. In some embodiments, the polynucleotide is an siRNA, mRNA, miRNA, or an anti-sense oligonucleotide. In some embodiments, the polynucleotide is an mRNA.

In some embodiments, the sugar or sugar alcohol is a sugar. In some embodiments, the sugar is trehalose, lactose, sucrose, or maltose. In some embodiments, the sugar is trehalose. In some embodiments, the buffer is a buffer with a pH range around 6-9. In some embodiments, the buffer is Tris, phosphate, or histidine. In some embodiments, the buffer is Tris.

In some embodiments, the lipid nanoparticle comprises one or more of a lipid, a sterol, a polymer conjugated lipid, and a phospholipid. In some embodiments, the lipid is a cationic lipid. In some embodiments, the lipid is a zwitterionic lipid. In some embodiments, the lipid is an anionic lipid. In some embodiments, the sterol is cholesterol. In some embodiments, the polymer conjugated lipid is a PEGylated lipid.

In some embodiments, the pharmaceutical composition comprises from about 50% to about 95% by weight of the sugar of sugar alcohol. In some embodiments, the weight of the sugar or sugar alcohol is from about 2% to about 25%. In some embodiments, the weight of the sugar or sugar alcohol is from about 2.5% to about 20%. In some embodiments, the methods result in delivery of the biologic active agent to the middle turbinate of at least 10%. In some embodiments, at least 15% of the biologic active agent is delivered to the middle turbinate. In some embodiments, at least 20% of the biologic active agent is delivered to the middle turbinate.

In some embodiments, the methods result in delivery of the biologic active agent to the lower turbinate of at least 10%. In some embodiments, at least 15% of the biologic active agent is delivered to the lower turbinate. In some embodiments, at least 20% of the biologic active agent is delivered to the lower turbinate.

In some embodiments, the methods result in delivery of the biologic active agent to the nasopharynx of at least 10%. In some embodiments, at least 15% of the biologic active agent is delivered to the nasopharynx. In some embodiments, at least 20% of the biologic active agent is delivered to the nasopharynx.

In some embodiments, the methods result in delivery of the biologic active agent of at least 25% to the middle turbinate, lower turbinate, and the nasopharynx. In some embodiments, at least 40% of the biologic active agent is delivered to the middle turbinate, lower turbinate, and the nasopharynx. In some embodiments, at least 50% of the biologic active agent is delivered to the middle turbinate, lower turbinate, and the nasopharynx.

In still yet another aspect, the present disclosure provides methods of preparing a pharmaceutical composition comprising:
(A) dissolving a precursor solution in a solvent to obtain a pharmaceutical mixture; wherein the precursor solution comprises:
   (1) a biologic active agent;
   (II) a sugar or sugar alcohol; and
   (III) a buffer;
(B) applying the pharmaceutical mixture to a surface at a surface temperature below 0° C. to obtain a frozen pharmaceutical mixture; wherein the pharmaceutical mixture comprises at least about 3% by weight of the precursor solution; and
(C) collecting the frozen pharmaceutical mixture and drying the frozen pharmaceutical mixture to obtain a pharmaceutical composition.

In some embodiments, the solvent is water. In some embodiments, the solvent is water that is free from any nucleases. In some embodiments, the pharmaceutical mixture is admixed until the pharmaceutical mixture is clear. In some embodiments, the pharmaceutical mixture comprises a solid content from about 3% w/v to about 15% w/v of the precursor solution. In some embodiments, the solid content is from about 3% w/v to about 7.5% w/v of the precursor solution. In some embodiments, the solid content is from about 3% w/v to about 5% w/v of the precursor solution.

In some embodiments, the pharmaceutical mixture is applied with a nozzle such as a needle.

In some embodiments, the pharmaceutical mixture is applied from a height from about 2 cm to about 50 cm. In some embodiments, the height is from about 5 cm to about 20 cm. In some embodiments, the height is about 10 cm. In some embodiments, the surface temperature is from about 0° C. to −190° C. In some embodiments, the surface temperature is from about −25° C. to about −125° C. In some embodiments, the surface temperature is about −100° C.

In some embodiments, the surface is a rotating surface. In some embodiments, the surface is rotating at a speed from about 5 rpm to about 500 rpm. In some embodiments, the surface is rotating at a speed from about 100 rpm to about 400 rpm. In some embodiments, the surface is rotating at a speed of about 200 rpm.

In some embodiments, the frozen pharmaceutical composition is dried by lyophilization. In some embodiments, the frozen pharmaceutical composition is dried at a first reduced pressure. In some embodiments, the first reduced pressure is from about 10 mTorr to 500 mTorr. In some embodiments, the first reduced pressure is from about 50 mTorr to about 250 mTorr. In some embodiments, the first reduced pressure is about 80 mTorr. In some embodiments, the frozen pharmaceutical composition is dried at a first reduced temperature. In some embodiments, the first reduced temperature is from about 0° C. to −100° C. In some embodiments, the first reduced temperature is from about −20° C. to about −60° C. In some embodiments, the first reduced temperature is about −40° C.

In some embodiments, the frozen pharmaceutical composition is dried for a primary drying time period from about 3 hours to about 2 weeks. In some embodiments, the primary drying time period is from about 6 hours to about 36 hours. In some embodiments, the primary drying time period is about 20 hours. In some embodiments, the frozen pharmaceutical composition is dried a secondary drying time period. In some embodiments, the frozen pharmaceutical composition is dried a secondary drying time at a second reduced pressure. In some embodiments, the secondary drying time is at a reduced pressure is from about 10 mTorr to 500 mTorr. In some embodiments, the secondary drying time is at a reduced pressure is from about 50 mTorr to about 250 mTorr. In some embodiments, the secondary drying time is at a reduced pressure is about 100 mTorr. In some embodiments, the frozen pharmaceutical composition is dried a secondary drying time at a second temperature. In some embodiments, the second temperature is from about 0° C. to 30° C. In some embodiments, the second temperature is from about 10° C. to about 30° C. In some embodiments, the second temperature is about 25° C. In some embodiments, the frozen pharmaceutical composition is dried for a second time for a second time period from about 3 hours to about 2 weeks. In some embodiments, the second time period is from about 6 hours to about 36 hours. In some embodiments, the second time period is about 20 hours. In some embodiments, the temperature is changed from the first reduced temperature to the second reduced temperature over a ramping time period. In some embodiments, the ramping time period is from about 3 hours to about 2 weeks. In some embodiments, the ramping time period is from about 6 hours to about 36 hours.

In some embodiments, the ramping time period is about 20 hours.

In still another aspect, the present disclosure provides pharmaceutical compositions prepared using the methods described herein.

In yet another aspect, the present disclosure provides pharmaceutical compositions comprising:
(1) a biologic active agent; wherein the biologic active agent is an antibody;
(II) a sugar alcohol; wherein the sugar alcohol is mannitol; and
(III) a buffer; wherein the buffer is selected from Tris, phosphate, or histidine;
wherein the pharmaceutical composition comprises a plurality of drug particles having an average diameter from about 50 μm to about 1 cm.

In still another aspect, the present disclosure provides pharmaceutical compositions comprising:
(1) a biologic active agent; wherein the biologic active agent is an polynucleotide;
(II) a sugar; wherein the sugar is lactose;
(III) an excipient, wherein the excipient is an amino acid; and
(IV) a buffer; wherein the buffer is selected from Tris; and the buffer further comprise a chelating agent;
wherein the pharmaceutical composition comprises a plurality of drug particles having an average diameter from about 50 μm to about 1 cm.

In another aspect, the present disclosure provides pharmaceutical compositions comprising:
(1) a biologic active agent; wherein the biologic active agent is a polynucleotide encapsulated in a lipid nanoparticle; wherein the lipid nanoparticle comprises a lipid, a sterol, a phospholipid, or a polymer conjugated lipid;
(II) a sugar alcohol; wherein the sugar is trehalose; and
(III) a buffer; wherein the buffer is selected from Tris, phosphate, or histidine;
wherein the pharmaceutical composition comprises a plurality of drug particles having an average diameter from about 50 μm to about 1 cm.

In still yet another aspect, the present disclosure provides methods of delivering a polynucleotide or a peptide-based biologic to the lungs of a patient comprising:
(A) obtaining a pharmaceutical composition comprising:
   (1) a polynucleotide, an organism, or a peptide based biologic agent;
   (II) a sugar or sugar alcohol; and
   (III) a buffer;
(B) delivering the polynucleotide, organism, or peptide based biologic agent to the lung by ejecting the powder from a device into the lung.

In some embodiments, at least 40% of the polynucleotide or peptide-based biologic is delivered to the lungs. In some embodiments, at least 50% of the polynucleotide or peptide-based biologic is delivered to the lungs. In some embodiments, at least 60% of the polynucleotide or peptide-based biologic is delivered to the lungs.

In some embodiments, the peptide-based biologic is a peptide. In other embodiments, the peptide-based biologic is a protein. In other embodiments, the peptide-based biologic is a modified protein. In other embodiments, the peptide-based biologic is an enzyme. In other embodiments, the peptide-based biologic is a protein-drug conjugate. In some embodiments, the organism is a virus. In other embodiments, the organism is a bacterium. In other embodiments, the organism is a bacteriophage. In some embodiments, the polynucleotide is a therapeutic polynucleotide. In some embodiments, the therapeutic polynucleotide is an oligooxynucleotide. In some embodiments, the polynucleotide is an oligonucleotide. In some embodiments, the polynucleotide is a class A oligonucleotide. In other embodiments, the polynucleotide is a class B oligonucleotide. In other embodiments, the polynucleotide is a class C oligonucleotide. In some embodiments, the oligonucleotide contains cytosine-phosphate-guanine (CpG) motifs. In some embodiments, the polynucleotide is a polyuracil or a polyadenosine. In some embodiments, the polynucleotide is a polyuracil. In some embodiments, the polynucleotide is a polyadenoesine. In some embodiments, the polynucleotide is a combination of polyuracil and polyadenosine.

In some embodiments, the sugar or sugar alcohol is a sugar. In some embodiments, the sugar is trehalose, mannitol, lactose, sucrose, or maltose. In some embodiments, the sugar is lactose. In other embodiments, the sugar is trehalose. In some embodiments, the pharmaceutical composition further comprises an excipient. In some embodiments, the excipient is an amino acid. In some embodiments, the amino acid is a non-ionizable amino acid. In some embodiments, the amino acid is leucine.

In some embodiments, the buffer is a buffer with a pH range around 6-9. In some embodiments, the buffer is Tris, phosphate, or histidine. In some embodiments, the buffer is Tris. In some embodiments, the buffer further comprises a chelating compound. In some embodiments, the chelating compound is polycarboxylic acid such as EDTA.

In some embodiments, the pharmaceutical composition further comprises a second therapeutic agent. In some embodiments, the second therapeutic agent is an antigen.

In some embodiments, the weight ratio of the sugar or sugar alcohol and the excipient is from about 5:1 to about 1:3. In some embodiments, the weight ratio is from about 3:1 to about 1:1. In some embodiments, the weight ratio is about 3:2. In some embodiments, the weight ratio of the sugar or sugar alcohol and the polynucleotide is from about 20:1 to about 250:1.

In still yet another aspect, the present disclosure provides pharmaceutical compositions comprising:
(A) a polynucleotide; wherein the polynucleotide is a homopolymer of one nucleotide or an oligonucleotide;
(B) a sugar or sugar alcohol; and
(C) a buffer.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A & 1B. Particle size of AdjLMQ/OVA with different concentrations of mucoadhesive agents, (A) before and (B) after being subjected to TFFD.

FIGS. 2A & 2B. The particle size distribution and average particle size of the CMC containing AdjLMQ/OVA compositions before and after being subjected to TFFD.

FIG. 3. The SDS-PAGE results of AdjLMQ/OVA with 0%, 1.9% to 3.7% of CMC before and after TFFD.

FIGS. 4A & 4B. In vitro mucoadhesion test. (A) Experimental design. (B) Downward movement of TFF AdjLMQ/OVA vaccine powders with 0, 1.9 or 3.7% w/w CMC on 1.5% agar plus 2% porcine mucin gel (bars). Data are mean±S.D. (n=10). Data in B were analyzed using one-way ANOVA followed by Tukey's post hoc analysis. ***p<0.001.

FIGS. 5A & 5B. Representative SEM images of thin-film freeze-dried AdjLMQ/OVA (A) and AdjLMQ/OVA with 1.9% CMC (B).

FIGS. 6A & 6B. The deposition pattern of the TFFD AdjLMQ/OVA-FITC with 1.9% CMC in nasal casts based on the CT-scan of a male of 48 years of age (A) and a female of 7 years of age (B). Upper, middle, and lower mean upper, middle, and lower turbinates, respectively.

FIG. 7. The regions of the nasal cavity are shown including the anterior region, the upper, middle, and lower turbinate, and the nasopharynx region.

FIG. 8. SEC chromatograms of the AUG3387 mAbs in liquid or reconstituted from TFF mAb powders. The experiment was repeated three time with similar results.

FIGS. 9A-9D. The MFI results of the mAb formulations before being subjected to TFFD (A), and the reconstituted TFF mAb powders with different solid contents (B), (C), (D). The concentrations of the mAb in each sample were 0.3104 mg/mL. Data are mean±S.D. for all the panels.

FIG. 10. Calculated mass percentage of subvisible particles in different AUG3387 mAb formulations (i.e., with the E-V method). Data are mean±S.D. (n=3).

FIG. 11. The XRD diffractograms of the TFFD mAb powders and the simulated patterns of mannitol (S form) (CCDC: 224660), leucine (CCDC: 1206031), and L-histidine (COD: 5000009).

FIG. 12. A representative SEM image of TFF AUG-3387C powder.

FIGS. 13A-B. Deposition pattern of TFF AUG-3387C powder in the nasal replica cast based on the CT-scan of (A) a 48-year-old male and (B) a 7-year-old female. Data are mean with range (n=2).

FIGS. 14A-D. Deposition patterns of the CpG 1826 ODN/OVA model vaccine dry powder prepared by thin-film freeze-drying. (A) A photograph of nasal cast 3D-printed based on the CT-scan images of the nasal cavities of a 48-year-old male showing different regions of human nasal cavity. (B) Deposition pattern of CpG 1826 ODN/OVA dry powder in the nasal replica cast of a 48-year-old male. (C) A photograph of nasal cast 3D-printed based on the CT-scan images of the nasal cavities of a 7-year-old female showing different regions of human nasal cavity. (D) Deposition pattern of CpG 1826 ODN/OVA dry powder in the nasal replica cast of a 7-year-old female. Data are mean±S.D. (n=3).

FIGS. 15A & B. Characterization of polyadenylic acid-lipid nanoparticles [poly(A)-LNPs]. (A) Mean particle size of poly(A)-LNPs containing various concentration of trehalose and (B) encapsulation efficiency of poly(A) in the LNPs before TFFD and after TFFD and reconstitution. Formulations containing different trehalose concentrations showed similar poly(A) encapsulation efficiency in the LNPs before TFFD. Data are mean±S.D. (n=3). ns: no significant difference between the means of different groups, ****p<0.0001.

FIGS. 16A & B. Deposition patterns of the poly(A)-LNP dry powder prepared using thin-film freeze-drying in different regions of (A) a nasal replica cast of a 48-year-old male, and (B) a nasal replica cast of a 7-year-old female. Data are represented as mean with range (n=2).

FIGS. 17A & 17B: In vitro aerosol performance of CpG 1826 ODN dry powders prepared using TFFD. Shown are the dry powder compositions with the best aerosol performance characteristics for deposition in the lungs. (A) The relative deposition of dry powders in various stages of the NGI. (B) The aerosol performance parameters of different dry powder compositions. Data presented are mean±S.D. of three independent experiments.

FIG. 18: Agarose gel electrophoresis of CpG 1826 ODNs in liquid formulations (i.e., before TFFD) and reconstituted from dry powders (i.e., after TFFD). TFF and reconstitution did not affect the integrity of CpG 1826 ODNs. Following TFFD and reconstitution, CpG 1826 ODNs were analyzed on 2% w/v agarose gel electrophoresis in the presence of ethidium bromide. Original CpG 1826 ODNs in Tris-EDTA and CpG 1826 ODNs in liquid formulations before TFFD were also analyzed as controls.

FIGS. 19A-C: Immunostimulatory capacity of representative CpG 1826 ODN dry powder compositions in J774A.1 murine macrophages. Cells (1×10⁴ cells/well) were treated with Tris-EDTA (TE) buffer, lipopolysaccharide (LPS, 100 ng/mL), CpG 1826 ODNs, or compositions 15 or 16 in liquid or reconstituted from dry powders at 2 μM of CpG 1826 ODNs for 24 h. The concentrations of TL-6 (A), IL-12 (B) and TNF-α (C) in the culture supernatants of J774A.1 murine macrophages were determined. Data presented are mean±S.D. of three independent experiments. Statistical significance between different groups was ascertained using one-way ANOVA followed by Tukey's post-hoc test where *p<0.05; ns: non-significant.

FIG. 20: PXRD patterns of CpG 1826 dry powder compositions 15 and 16 comprising lactose or trehalose as a stabilizer, respectively, at a concentration of 0.48% w/w prepared using TFFD. Shown also the PXRD patterns of simulated leucine, simulated lactose, and simulated trehalose.

FIG. 21: Representative scanning electron micrographs of CpG 1826 dry powder compositions 15 and 16 prepared using lactose or trehalose as a stabilizer, respectively, at a concentration of 0.48% w/w. The bar represents 3 μm (magnification: 15,000×).

FIGS. 22A & 22B: In vitro aerosol performance of dry powder compositions comprising various TLR agonists prepared using TFFD. (A) The relative deposition of dry powders in various stages of the NGI. (B) The aerosol performance properties of different dry powder compositions. Data in A are mean with range of two independent experiments.

FIGS. 23A & 23B: In vitro aerosol performance of CpG 1826 ODN-adjuvanted OVA model vaccine dry powder compositions prepared using TFFD. (A) The relative deposition of dry powders in various stages of the NGI. (B) The aerosol performance properties of different dry powder compositions. Data presented are mean±S.D. of three independent experiments.

### DETAILED DESCRIPTION

Thin-film freeze-drying (TFFD) is a dry powder engineering technology. It involves the ultra-rapid freezing of a liquid (e.g., solution, suspension, or emulsion) on a cryogenically cooled solid surface. The liquid is dropped from a distance of about 1 cm to 10 cm above the cryogenically cooled solid surface (e.g., a rotating metal drum, the inner bottom of a precooled glass vial). The diameters of the droplets are about 2 mm. Upon impact on the cooled surface, the droplet rapidly spreads to a thin film, which is then frozen to a frozen thin film. Solvent such as water in the frozen thin film is removed by lyophilization. TFFD is advantageous over other dry powder engineering technology such as conventional shelf freeze-drying, spray drying, and spray freeze-drying in that it avoids or minimizes shear stress and heat stress, while the powders generated by TFFD are generally highly porous, brittle, and having large specific surface areas, making them potentially feasible for direct intranasal administration without reconstitution. Previously, the inventors have demonstrated that TFFD can be applied to prepare dry powders of various vaccines, including vaccines adjuvanted with aluminum salts, liposomes, or (nano)emulsions.

Herein, using a model vaccine comprised of ovalbumin (OVA) as a model antigen and the liposomal adjuvant formulation that contains MPLA and QS21 (AdjLMQ) as an adjuvant, the inventors disclose compositions of vaccine dry powders for direct intranasal administration and the method of preparing them. For direct intranasal delivery of the dry powders of the AdjLMQ-adjuvanted vaccine, the inventors tested adding mucoadhesive agents into the vaccine dry powders to render them mucoadhesive, with the intention of increasing the residence time of the vaccine in the nasal cavity upon intranasal administration. Mucoadhesive agents tested include chitosan, sodium alginate, gelatin, and sodium carboxymethylcellulose (CMC). Each of the mucoadhesive agents has its own unique mechanism(s) of interaction with the nasal mucosal surface (Sogias et al., 2008; Kesavan et al., 2010; Grabovac et al., 2005; Dekina et al., 2016). A challenge is that the mucoadhesive agents could interact with the AdjLMQ/OVA vaccine and cause a change in the structure of the AdjLMQ and/or the vaccine candidate, and thus affecting the efficacy of the AdjLMQ/OVA vaccine. Therefore, the inventors studied the effect of the mucoadhesive agents and their concentrations on the AdjLMQ/OVA vaccine integrity before and after being subjected to TFFD. After characterization of a selected dry powder, the inventors demonstrated that the new thin-film freeze-dried powder, when sprayed using a powder sprayer at an initial powder velocity of about 200 cm/s, showed a desirable deposition pattern in nasal casts 3D printed based on the CT-scan images of human noses.

Furthermore, the thin-film freeze-drying technology may be used to prepare dry powders of biologic compositions, such as monoclonal antibodies and nucleic acids suitable for intranasal delivery. More particularly, the inventors disclose dry powder compositions of biologics and nucleic acid-based products (e.g., monoclonal antibodies, CpG oligos (mixed with a protein antigen), mRNA-LNPs) prepared by thin-film freeze-drying. Besides the active ingredient(s) (i.e., a biologic or a nucleic acid-based product), the dry powders are comprised of a sugar or sugar alcohol, an amino acid, a protein, polymer, buffer and/or other excipients. The dry powders can be directly administered to the nasal cavity with a dry powder spraying device or the lung using a dry powder inhaler device, depending on the compositions. In the examples provided, the targeted region of the nasal cavity in the posterior nasal cavity. However, one may apply the powders to the upper turbinate region in the nasal cavity to target the olfactory region, if needed.

### I. Definitions

The abbreviations used herein have their conventional meaning within the chemical and biological arts. Description of compounds of the present disclosure is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions.

The terms “a” or “an,” as used in herein means one or more. In addition, the phrase “substituted with a[n],” as used herein, means the specified group may be substituted with one or more of any or all of the named substituents. The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination, neuropsychiatric exams, and/or a psychiatric evaluation. For example, the certain methods presented herein successfully treat a disease associated with (e.g., caused by) an infectious agent (e.g., bacterium or virus). The term “treating” and conjugations thereof, include prevention of an injury, pathology, condition, or disease. The term “preventing” or “prevention” refers to any indicia of success in protecting a subject or patient (e.g., a subject or patient at risk of developing a disease or condition) from developing, contracting, or having a disease or condition (e.g., an infectious disease or diseases associated with an infectious agent), including preventing one or more symptoms of a disease or condition or diminishing the occurrence, severity, or duration of any symptoms of a disease or condition following administration of a prophylactic or preventative composition as described herein.

An “effective amount” is an amount sufficient for a composition (e.g., compound, vector, drug) to accomplish a stated purpose relative to the absence of the composition (e.g., compound, vector, drug) (e.g., achieve the effect for which it is administered, treat a disease (e.g., reverse or prevent or reduce severity), reduce spread of an infectious disease or agent, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a composition is an amount of a composition that, when administered to a subject, will have the intended prophylactic effect, e.g., preventing or delaying the onset (or reoccurrence) of an injury, disease (e.g., infectious disease), pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses (e.g., prime-boost). Thus, a prophylactically effective amount may be administered in one or more administrations. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of infection or one or more symptoms of infection in the absence of a composition as described herein (including embodiments).

“Contacting” is used in accordance with its plain ordinary meaning and refers to the process of allowing at least two distinct species (e.g., compositions, vectors, bacterium, virus, biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a composition as described herein and a cell, virus, virus particle, protein, enzyme, or patient. In some embodiments contacting includes allowing a composition described herein to interact with a protein or enzyme that is involved in a signaling pathway. In some embodiments contacting includes allowing a composition described herein to interact with a component of a subject's immune system involved in developing immunity to a component of the composition.

As defined herein, the term “inhibition”, “inhibit”, “inhibiting” and the like in reference to a protein-inhibitor or interaction means negatively affecting (e.g., decreasing) the activity or function of the protein. In some embodiments, inhibition refers to reduction of a disease or symptoms of disease. In some embodiments, inhibition refers to reduction of the growth, proliferation, or spread of an infectious agent (e.g., bacterium or virus). In some embodiments, inhibition refers to preventing the infection of a subject by an infectious agent (e.g., bacterium or virus). In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or downregulating the signaling pathway or enzymatic activity or the amount of a protein.

The term “modulator” refers to a composition that increases or decreases the level of a target (e.g., molecule, cell, bacterium, virus particle, protein) or the function of a target or the physical state of the target. The term “modulate” is used in accordance with its plain ordinary meaning and refers to the act of changing or varying one or more properties. “Modulation” refers to the process of changing or varying one or more properties. For example, as applied to the effects of a modulator on a target, to modulate means to change by increasing or decreasing a property or function of the target or the amount of the target.

“Patient” or “subject in need thereof” refers to a living organism suffering from or prone to a disease or condition that can be treated by administration of a composition (e.g., pharmaceutical composition) as provided herein. Non-limiting examples include humans, other mammals, bovines, rats, mice, dogs, monkeys, goat, sheep, cows, deer, and other non-mammalian animals. In some embodiments, a patient is human. In some embodiments, a patient or subject in need thereof, refers to a living organism (e.g., human) at risk of developing, contracting, or having a disease or condition associated with an infectious agent (e.g., bacterium or virus).

“Disease” or “condition” refer to a state of being or health status of a patient or subject capable of being treated with the compositions or methods provided herein. In some embodiments, the disease is a disease related to (e.g., caused by) an infectious agent (e.g., bacterium or virus).

“Pharmaceutically acceptable excipient” and “pharmaceutically acceptable carrier” refer to a substance that aids the administration of an active agent to or absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure. In embodiments, an excipient is a salt, buffer, detergent, polymer, amino acid, or preservative. In embodiments, the excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benzyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

The term “preparation” is intended to include the formulation of the active compound with encapsulating material as a carrier providing a capsule in which the active component with or without other carriers, is surrounded by a carrier, which is thus in association with it.

As used herein, the term “administering” means respiratory administration. Such administration could be to the nasal cavity (intranasal administration) or to the lungs (pulmonary administration). By “co-administer” it is meant that a composition described herein is administered at the same time, just prior to, or just after the administration of one or more additional therapies, for example infection therapies such as antiviral drugs or antibiotics. The compositions of the disclosure can be administered alone or can be co-administered to the patient. Coadministration is meant to include simultaneous or sequential administration of the compounds individually or in combination (more than one composition). Thus, the preparations can also be combined, when desired, with other active substances. The compositions of the present disclosure can be delivered by transdermally, by a topical route, transcutaneously, formulated as solutions, suspensions, emulsions, gels, creams, ointments, pastes, jellies, paints, powders, and aerosols.

The terms “peptide,” “polypeptide,” and “protein” are used interchangeably herein to refer to a polymer of amino acid residues. The term “peptidyl” and “peptidyl moiety” means a monovalent peptide.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. An oligomer comprising amino acid mimetics is a peptidomimetic. A peptidomimetic moiety is a monovalent peptidomimetic.

The term “isolated” refers to a nucleic acid, polynucleotide, polypeptide, protein, or other component that is partially or completely separated from components with which it is normally associated (other proteins, nucleic acids, cells, etc.). In some embodiments, an isolated polypeptide or protein is a recombinant polypeptide or protein.

The terms “dose” and “dosage” are used interchangeably herein. A dose refers to the amount of active ingredient given to an individual at each administration. For the present methods and compositions provided herein, the dose may generally refer to the amount of disease treatment. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition and depends on the route of administration. For example, a dosage form can be in a liquid form for nebulization, e.g., for inhalants, in a tablet or liquid, e.g., for oral delivery, or a saline solution, e.g., for injection.

The terms “bind”, “bound”, “binding”, and other verb forms thereof are used in accordance with their plain ordinary meaning within Enzymology and Biochemistry and refer to the formation of one or more interactions or contacts between two compositions that may optionally interact. Binding may be intermolecular or intramolecular.

The term “adjuvant” is used in accordance with its plain ordinary meaning within Immunology and refers to a substance that is commonly used as a component of a vaccine. Adjuvants may increase an antigen specific immune response in a subject when administered to the subject with one or more specific antigens as part of a vaccine. In some embodiments, an adjuvant accelerates an immune response to an antigen. In some embodiments, an adjuvant prolongs an immune response to an antigen. In some embodiments, an adjuvant enhances an immune response to an antigen. In some embodiments, an adjuvant is a squalene-based adjuvant. In some embodiments, an adjuvant is a liposome formulation containing MPLA and QS21. In some embodiments, an adjuvant is a CpG oligo. In yet other embodiments, an adjuvant is a nanoparticle such as the lipid nanoparticles (LNPs).

The term “vaccine” is used according to its plain ordinary meaning within medicine and Immunology and refers to a composition including an antigenic component (e.g., antigenic protein) for administration to a subject (e.g., human), which elicits an immune response to the antigenic component (e.g., antigenic protein). In some embodiments a vaccine is a therapeutic. In some embodiments, a vaccine is prophylactic. In some embodiments a vaccine includes one or more adjuvants (e.g., squalene-based adjuvant). A liquid vaccine is a vaccine in liquid form, which may be for example a solution, suspension, emulsion, or dispersion or the antigenic component (e.g., antigenic protein) of the vaccine and may optionally include other components. A dry vaccine is a vaccine comprising 5% or less of water.

A vaccine is a preparation employed to improve immunity to a particular disease. Vaccines include an agent, which is used to induce a response from the immune system of the subject. Various agents that are typically used in a vaccine include, but are not limited to: killed, but previously virulent, micro-organisms; live, attenuated microorganisms; inactivated toxic compounds that are produced by microorganism that cause an illness; protein subunits of microorganisms; conjugates; and nucleic acid-based vaccines such as plasmid DNA vaccines and messenger RNA vaccines. Examples of vaccines that may be converted into a powder vaccine according to the methods described herein include, but are not limited to: influenza vaccine, cholera vaccine, bubonic plague vaccine, polio vaccine, Hepatitis A vaccine, rabies vaccine, yellow fever vaccine, measles vaccine, rubella vaccine, mumps vaccine, typhoid vaccine, tuberculosis vaccine, tetanus vaccine, diphtheria vaccine, diphtheria-tetanus-pertussis vaccine, Hepatitis B vaccine, human papillomavirus (HPV) vaccine, Pneumococcal conjugate vaccines, influenza vaccine, botulism vaccine, polio vaccine, anthrax vaccines, and Coronavirus vaccines.

The term “prime-boost” or “prime boost” as applied to a methodology of administering vaccines is used according to its plain ordinary meaning in Virology and Immunology and refers to a method of vaccine administration in which a first dose of a vaccine or vaccine component is administered to a subject or patient to begin the administration (prime) and at a later time (e.g., hours, days, weeks, months later) a second vaccine is administered to the same patient or subject (boost). The first and second vaccines may be the same or different but are intended to both elicit an immune response useful in treating or preventing the same disease or condition. In some embodiments the prime is one or more viral proteins or portions thereof and the boost is one or more viral proteins or portions thereof.

The term “associated” or “associated with” as used herein to describe a disease means that the disease is caused by, or a symptom of the disease is caused by, what is described as disease associated or what is described as associated with the disease. As used herein, what is described as being associated with a disease, if a causative agent, could be a target for treatment of the disease.

The term “initial velocity” refers to the speed by which the powder is actuating/flying out the delivery device. In some embodiments, the initial velocity is at least 200 cm/s, 225 cm/s, 250 cm/s, 275 cm/s, 300 cm/s, 325 cm/s, 350 cm/s, 375 cm/s, 400 cm/s, 450 cm/s, 500 cm/s, 600 cm/s, 700 cm/s, 800 cm/s, 900 cm/s, 1000 cm/s, 1250 cm/s, 1500 cm/s, 1750 cm/s, 2000 cm/s, 2250 cm/s, 2500 cm/s, 2750 cm/s, 3000 cm/s, 3250 cm/s, 3500 cm/s, 3750 cm/s, or 4000 cm/s.

The term “portion” refers to a subset of a whole, which may also be the whole. In some embodiments, a portion is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In some embodiments, a portion is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. Unless indicated otherwise, the term “about” in the context of a numeric value indicates the nominal value±10% of the nominal value. In some embodiments, “about” may be the nominal value.

### II. Vaccine Compositions

In one aspect there is provided a dry powder of a liposomal formulation. In some aspects, the formulations will contain an additional agent, such as a therapeutic or diagnostic agent. The therapeutic agent may be a biological or a pharmaceutical drug. A biological may be a protein, such as an antibody, a peptide, an aptamer, an oligonucleotide, a polynucleotide, or an expression construct, such as a viral expression construct. A pharmaceutical drug may be an anti-cancer drug, such as a chemotherapeutic, an anti-viral drug, an anti-fungal drug, an antibiotic/antibacterial drug, a drug that modulates one or more aspects of cardiovascular disease, a mental health disorder, diabetes, a pulmonary disease, kidney disease, or an autoimmune disease. In some aspects, the present disclosure relates to a vaccine or a vaccine formulation.

In embodiments, the dry formulation includes less than 5% water. In embodiments, the dry formulation includes less than 4% water. In embodiments, the dry formulation includes less than 3% water. In embodiments, the dry formulation includes less than 2% water. In embodiments, the dry formulation includes less than 1% water. In embodiments, the dry formulation includes less than 5% water (wt/wt). In embodiments, the dry formulation includes less than 4% water (wt/wt). In embodiments, the dry formulation includes less than 3% water (wt/wt). In embodiments, the dry formulation includes less than 2% water (wt/wt). In embodiments, the dry formulation includes less than 1% water (wt/wt). In embodiments, the dry formulation includes about 5% water. In embodiments, the dry formulation includes about 4% water. In embodiments, the dry formulation includes about 3% water. In embodiments, the dry formulation includes about 2% water. In embodiments, the dry formulation includes about 1% water. In embodiments, the dry formulation includes about 5% water (wt/wt). In embodiments, the dry formulation includes about 4% water (wt/wt). In embodiments, the dry formulation includes about 3% water (wt/wt). In embodiments, the dry formulation includes about 2% water (wt/wt). In embodiments, the dry formulation includes about 1% water (wt/wt). In embodiments, the dry formulation includes less than 5% water (v/v). In embodiments, the dry formulation includes less than 4% water (v/v). In embodiments, the dry formulation includes less than 3% water (v/v). In embodiments, the dry formulation includes less than 2% water (v/v). In embodiments, the dry formulation includes less than 1% water (v/v). In embodiments, the dry formulation includes about 5% water (v/v). In embodiments, the dry formulation includes about 4% water (v/v). In embodiments, the dry formulation includes about 3% water (v/v). In embodiments, the dry formulation includes about 2% water (v/v). In embodiments, the dry formulation includes about 1% water (v/v).

In embodiments, the dry formulation includes an excipient. In embodiments, the dry formulation includes a plurality of different excipients. In embodiments, the excipient is a salt, buffer, detergent, polymer, amino acid, or preservative. In embodiments, the excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. In embodiments, the excipient is trehalose. In embodiments, the dry formulation includes about 60% or above, wt/wt, of the excipient. In embodiments, the dry formulation includes less than 4% wt/wt of the excipient. In embodiments, the dry formulation includes less than 3% wt/wt of the excipient. In embodiments, the dry formulation includes less than 2% wt/wt of the excipient. In embodiments, the dry formulation includes less than 1% wt/wt of the excipient. In embodiments, the dry formulation includes less than 0.5% wt/wt of the excipient. In embodiments, the dry formulation includes about 5% wt/wt of the excipient. In embodiments, the dry formulation includes about 4% wt/wt of the excipient. In embodiments, the dry formulation includes about 3% wt/wt of the excipient. In embodiments, the dry formulation includes about 2% wt/wt of the excipient. In embodiments, the dry formulation includes about 1% wt/wt of the excipient. In embodiments, the dry formulation includes about 0.5% wt/wt of the excipient.

In embodiments, the dry formulation includes liposomal particles. In embodiments, the dry formulation is prepared from a liquid formulation.

In an embodiment, a powder (e.g., dry) formulation, which retains its efficacy, may be made from a liquid composition. The method includes obtaining a liquid (e.g., aqueous) composition. The liposomal composition may be frozen to obtain a frozen composition (e.g., thin film). Water is removed from the frozen composition to form a powder (e.g., dry) that includes the agent or compound.

A cryoprotectant may be added to the liposomal composition to protect the agents present in the composition (either live or dead) from damage during the freezing process. Examples of cryoprotectants include glycerol, monosaccharides, disaccharides, and polysaccharides (e.g., trehalose), polymers (e.g., PVP), amino acids (e.g., leucine), or proteins (e.g., human serum albumin). A cryoprotectant may be present in amounts up to about 90% by weight in the dry powder.

In another embodiment, an aqueous composition may be composed of an agent that forms particles having a particle size of less than about 500 nm (e.g., less than 50, 60, 70, 80, 90, 100, 110, 120, 130, 140, 150, 160, 170, 180, 190, 200, 225, 250, 275, 300, 350, 400, 450, or 500 nm). In some embodiments, particles having a diameter of less than 200 nm (e.g., less than 50, 60, 70, 80, 90, 100, 110, 120, 130, 140, 150, 160, 170, 180, 190, 200, 225, 250, 275, 300, 350, 400, 450, or 500 nm) may be used. In some embodiments, the aqueous composition can be converted to a powder, as described above, for storage, for use as an inhalant, or use in other delivery modes.

In embodiments, a dry formulation is the dry formulation described herein, including in embodiments, examples, tables, figures, and claims. In embodiments, a dry formulation is a dry formulation made by a method described herein, including in aspects, embodiments, examples, tables, figures, and claims. Provided herein is a reconstituted liquid formulation comprising a dry formulation as described herein (including in an aspect, embodiment, example, table, figure, or claim) or a dry formulation prepared using a method as described herein (including in an aspect, embodiment, example, table, figure, or claim) and a solvent (e.g., water, buffer, solution, liquid including an excipient).

Provided in another aspect is a pharmaceutical composition including a pharmaceutically acceptable excipient and any of the compositions described herein (including embodiments).

The compositions described herein (including embodiments and examples) can be administered alone or can be co-administered to the patient. Coadministration is meant to include simultaneous or sequential administration of the compositions individually or in combination (more than one composition). Thus, the preparations can also be combined, when desired, with other active substances.

Pharmaceutical compositions provided by the present disclosure include compositions wherein the active ingredient (e.g., compositions described herein, including embodiments) is contained in a therapeutically or prophylactically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., prevent infection, and/or reducing, eliminating, or slowing the progression of disease symptoms. Determination of a therapeutically or prophylactically effective amount of a composition of the disclosure is well within the capabilities of those skilled in the art, especially in light of the detailed disclosure herein.

### III. Biologics Compositions

The present disclosure provides compositions of biologically active antibodies that may be prepared using thin-film freezing (TFF). The TFF process is a cryogenic technology newly adapted to the pharmaceutical industry to engineer dry powders with good aerosol performance for pulmonary delivery. Previously, thin-film freezing (TFF) was successfully applied to prepare dry powders of proteins such as lysozyme and lactose dehydrogenase (LDH) while preserving their enzymatic activity, but the aerosol properties of the powders were unknown.

Provided herein are dry powder formulations of biologically active molecules that can be made by an ultra-rapid freezing (URF) process. The resulting dry powder formulations have a number of distinct advantages. For example, thin film freezing is an ultra-rapid freezing process (i.e., 100-1000 K/s) that can preserve particle size distribution via accelerating the nucleation rate and the formation of small ice crystals. Biologically active molecules, such as monoclonal antibodies and nucleic acids are dropped onto a cryogenically cooled surface to form frozen thin-films within, for example, 50 ms to 5 s. Exposure may comprise spraying or dripping droplets of said biologically active molecules. The freezing surface temperature may be about −190° C. to about 0° C., the diameters of the droplets are about 2-5 millimeters, and the droplets are dropped from a distance about 1 cm to 10 cm from the freezing surface. The method may comprise contacting the droplets with a freezing surface having a temperature differential of at least about 30° C. between the droplets and the surface. The freezing rate of said droplets may be between 10 K/second and 10³ K/second. The method may further comprise removing the solvent from the thin film to form a dry composition. Such as wherein said removing of the solvent comprises lyophilization/sublimation. Other high-speed freezing methods may also be employed. Technologies with slower freezing rate (e.g., conventional shelf freeze-drying) result in phase separation and large ice crystals and thus damage of proteins (e.g., denaturation and/or aggregation). It was shown that, by the use of URF, the compositions can be stabilized such that the mAbs are protected from excessive degradation and components retain substantial biological activity after formulation.

In some cases, formulations include a sugar, such as trehalose, to provide yet further stabilization. Furthermore, dry powders of the embodiments can comprise a wide variety of antibody-containing compositions. Moreover, it has been demonstrated that the powders of the embodiments can be used to directly administer therapeutic agents, e.g., to the lungs or the nasal cavity. Thus, the aspects of the present invention provide new pharmaceutical formulations, formulation methods and administration modalities that demonstrate significant advantages over previously compositions and methods that have been used.

In some embodiments, compositions of the present disclosure comprise mAbs, such as a mAb specific for SARS-CoV-2. It has been shown that mAbs processed into powders as detailed herein are able to retain substantial activity. See, for example, Hufnagel et al. 2021. Thus, methods and compositions provided herein can be used to stabilize mAbs, such as for storage and/or transportation. Likewise, mAb-containing powders can be directly administered to patients in need thereof (or reconstituted prior to administration).

A. Monoclonal Antibodies (mAbs) and Antibody Fragments

Methods and compositions of the embodiments concern biologically active antibodies. The term “antibody” refers to an intact immunoglobulin of any isotype or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, polyclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2 as either monomeric or as dimeric form. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), Chothia & Lesk, J. Mol. Biol., 196:901-917 (1987) or Chothia et al., Nature, 342:878-883 (1989).

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79: 315-321 (1990); Kostelny et al., J. Immunol., 148:1547-1553 (1992).

The term “antigen” refers to a substance capable of inducing adaptive immune responses. Specifically, an antigen is a substance which serves as a target for the receptors of an adaptive immune response. Typically, an antigen is a molecule that binds to antigen-specific receptors but cannot induce an immune response in the body by itself. Antigens are usually proteins and polysaccharides, less frequently also lipids. As used herein, antigens also include immunogens and haptens.

An “Fc” region comprises two heavy chain fragments comprising the CH1 and CH2 domains of an antibody. The two heavy chain fragments are held together by two or more disulfide bonds and by hydrophobic interactions of the CH3 domains.

The “Fv region” comprises the variable regions from both the heavy and light chains but lacks the constant regions.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The term “compete” when used in the context of antigen binding proteins (e.g., antibody or antigen-binding fragment thereof) that compete for the same epitope means competition between antigen binding proteins as determined by an assay in which the antigen binding protein (e.g., antibody or antigen-binding fragment thereof) being tested prevents or inhibits (e.g., reduces) specific binding of a reference antigen binding protein (e.g., a ligand, or a reference antibody) to a common antigen. Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

The term “epitope” as used herein refers to the specific group of atoms or amino acids on an antigen to which an antibody binds. The epitope can be either linear epitope or a conformational epitope. A linear epitope is formed by a continuous sequence of amino acids from the antigen and interacts with an antibody based on their primary structure. A conformational epitope, on the other hand, is composed of discontinuous sections of the antigen's amino acid sequence and interacts with the antibody based on the 3D structure of the antigen. In general, an epitope is approximately five or six amino acid in length. Two antibodies may bind the same epitope within an antigen if they exhibit competitive binding for the antigen.

In some embodiments, the antibody is a monoclonal antibody. In further embodiments, the antibody is an IgG antibody. In some embodiments, the antibody binds to SARS-CoV-2 spike protein. In some embodiments, the antibody is an anti-SARS-CoV-2 antibody.

In some aspects, the present disclosure provides pharmaceutical compositions comprising a dry powder comprising a plurality of drug particles; wherein each drug particle comprises:
(A) a biologic active agent; and
(B) a sugar or sugar alcohol.

In some embodiments, the pharmaceutical composition further comprises a buffer. In some embodiments, the buffer is a phosphate buffer, such as a phosphate-buffered saline. In some embodiments, the buffer is a histidine buffer. In some embodiments, the sugar is a disaccharide, such as lactose, trehalose, or sucrose. In some embodiments, the pharmaceutical composition further comprises an amino acid. In some embodiments, the amino acid is a canonical amino acid. In some embodiments, the amino acid is a non-polar amino acid, such as leucine. In some embodiments, the pharmaceutical composition further comprises a surface-active agent. In some embodiments, the surface active agent is Tween 20. In some embodiments, the surface active agent in Poloxomer 188. In some embodiments, the pharmaceutical composition comprises an antibody fragment, such as a nanobody or an Fab′. In some embodiments, the antibody is a monoclonal antibody. In some embodiments, the antibody is an IgG antibody. In some embodiments, the antibody binds to SARS-CoV-2 spike protein. In some embodiments, the antibody is an anti-SARS-CoV-2 antibody.

In some embodiments, the pharmaceutical composition comprises a weight ratio of the sugar or sugar alcohol to the amino acid from about 1:6 to about 20:1, from about 1:2 to about 8:1 of the sugar to the amino acid, from about 3:2 to about 3:1, or from about 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 to about 20:1, or any range derivable therein. In other embodiments, the pharmaceutical composition does not comprise an amino acid. In some embodiments, the pharmaceutical composition comprises a weight ratio from about 0.1% to about 80% of the antibody relative to the total excipients, from about 0.25% to about 2.5% of the antibody, from about 0.33% to about 1.5% of the antibody, or from about 0.1%, 0.15%, 0.2%, 0.25%, 0.5%, 1%, 1.5%, 2%, 2.5%, 5%, 10%, 15%, 20%, 30%, 40%, 50%, 60%, 70%, to about 80%, or any range derivable therein. In some embodiments, the weight ratio of the antibody is relative to the total excipients. In other embodiments, the weight ratio of the antibody is relative to the amount of sugar or sugar alcohol in the composition.

In some embodiments, at least 50% of the antibodies in the pharmaceutical composition is in the monomeric form after storage at a temperature for a time period. In some embodiments, the pharmaceutical composition comprises at least at 75% of the antibodies in monomeric form, at least at 80% of the antibodies in monomeric form, or from about 50%, 55%, 60%, 65%, 70%, 75%, 80%, 85%, 90%, 95%, to about 100%, or any range derivable therein. In some embodiments, the temperature is room temperature. In some embodiments, the temperature is from about −180° C. to about 20° C., from about −80° C. to about 10° C., from about −10° C. to about 5° C., from about 10° C. to about 50° C., from about 15° C. to about 45° C., from about 20° C. to about 40° C., or from about −180° C., −160° C., −140° C., −120° C., −100° C., −90° C., −80° C., −70° C., −60° C., −40° C., −30° C., −20° C., −10° C., −5° C., 0° C., 5° C., 10° C., 15° C., 20° C., 25° C., 30° C., 35° C., 40° C., 45° C., to about 50° C., or any range derivable therein. In some embodiments, the pharmaceutical composition has been dissolved in water. In some embodiments, the water is saline. In some embodiments, the water is phosphate buffered saline. In some embodiments, the water is a citrate buffer. In other embodiments, the water is a histidine buffer.

### B. Nucleic Acids

In some aspects of the present disclosure, the biologic compositions comprise one or more nucleic acids. In some embodiments, the biologic composition comprises one or more nucleic acids present in a weight ratio to the composition from about 5:1 to about 1:10,000. In some embodiments, the weight ratio of nucleic acid to composition is from about 5:1, 2.5:1, 1:1, 1:5, 1:10, 1:15, 1:20, 1:25, 1:30, 1:35, 1:40, 1:45, 1:50, 1:60, 1:70, 1:80, 1:90, 1:100, 1:250, 1:500, 1:750, 1:1000, 1:2500, 1:5000, 1:7500, 1:10000, or any range derivable therein. In addition, it should be clear that the present disclosure is not limited to the specific nucleic acids disclosed herein. The present disclosure is not limited in scope to any particular source, sequence, or type of nucleic acid, however, as one of ordinary skill in the art could readily identify related homologs in various other sources of the nucleic acid including nucleic acids from non-human species (e.g., mouse, rat, rabbit, dog, monkey, gibbon, chimp, ape, baboon, cow, pig, horse, sheep, cat and other species). It is contemplated that the nucleic acid used in the present disclosure can comprises a sequence based upon a naturally-occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally-occurring sequence. In another embodiment, the nucleic acid is a complementary sequence to a naturally occurring sequence, or complementary to 75%, 80%, 85%, 90%, 95% and 100%. Longer polynucleotides encoding 250, 500, 1000, 1212, 1500, 2000, 2500, 3000 or longer are contemplated herein.

The nucleic acid used herein may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present disclosure may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

The term messenger RNA (mRNA), as used herein, the term “messenger RNA (mRNA)” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some aspects, the polynucleotide may be a CpG or contain a CpG motif. A nucleic acid having a cytosine followed by a guanine linked by a phosphate bond in which the pyrimidine ring of the cytosine is unmethylated. The term “methylated CpG” refers to the methylation of the cytosine on the pyrimidine ring, usually occurring the 5-position of the pyrimidine ring. A CpG oligodeoxynucleotide is an oligodeoxynucleotide that is at least about ten nucleotides in length and includes an unmethylated CpG. CpG oligodeoxynucleotides include both D and K type oligodeoxynucleotides (see below) CpG oligodeoxynucleotides are single-stranded. The entire CpG oligodeoxynucleotide can be unmethylated or portions may be unmethylated. In one embodiment, at least the C of the 5′ CG 3′ is unmethylated.

In some embodiments, the nucleic acid comprises one or more antisense segments which inhibits expression of a gene or gene product. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

### 1. Modified Nucleobases

In some embodiments, the nucleic acids of the present disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties.

In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rₘ)(Rₙ), and O—CH₂—C(═O)—N(Rm)(Rn), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties (see, e.g., PCT International Application WO 2008/101157, for additional 5′,2′-bis substituted sugar moieties and nucleosides).

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂-2′, 4′-(CH₂)₂-2′, 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845); 4′-C(CH₃)(CH₃)—O-2′ and analogs thereof, (see, e.g., WO 2009/006478); 4′-CH₂—N(OCH₃)-2′ and analogs thereof (see, e.g., WO2008/150729); 4′-CH₂—O—N(CH₃)-2′ (see, e.g., US2004/0171570, published Sep. 2, 2004); 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ and analogs thereof (see, PCT International Application WO 2008/154401).

In some embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH2-N(R)-2′) BNA, (I) methyl carbocyclic (4′-CH₂—CH(CH₃)-2′) BNA, (J) propylene carbocyclic (4′-(CH₂)₃-2′) BNA, and (K) Methoxy(ethyleneoxy) (4′-CH(CH₂OMe)-O-2′) BNA (also referred to as constrained MOE or cMOE).

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 5561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US 2004/0171570, US 2007/0287831, and US 2008/0039618; U.S. Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In some embodiments, bicyclic sugar moieties and nucleosides incorporating such bicyclic sugar moieties are further defined by isomeric configuration. For example, a nucleoside comprising a 4′-2′ methylene-oxy bridge, may be in the .alpha.-L configuration or in the .beta.-D configuration. Previously, α-L-methyleneoxy (4′-CH₂—O-2′) bicyclic nucleosides have been incorporated into antisense oligonucleotides that showed antisense activity (Frieden et al., Nucleic Acids Research, 2003, 21, 6365-6372).

In some embodiments, substituted sugar moieties comprise one or more non-bridging sugar substituent and one or more bridging sugar substituent (e.g., 5′-substituted and 4′-2′ bridged sugars; PCT International Application WO 2007/134181, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfer, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position (see, e.g., published U.S. Patent Application US 2005/0130923) and/or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al., J. Org. Chem., 2006, 71, 7731-7740).

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA) (see Leumann, C J. Bioorg. & Med. Chem. (2002) 10:841-854), and fluoro HNA (F-HNA).

In some embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In some embodiments, at least one of q₁, q₂, q₃, q₄, q₅, q₆ and q₇ is methyl. In some embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds (see, e.g., review article: Leumann, J. C, Bioorganic & Medicinal Chemistry, 2002, 10, 841-854).

Combinations of modifications are also provided without limitation, such as 2′-F-5′-methyl substituted nucleosides (see PCT International Application WO 2008/101157 for other disclosed 5′,2′-bis substituted nucleosides) and replacement of the ribosyl ring oxygen atom with S and further substitution at the 2′-position (see U.S. Patent Publication US 2005/0130923) or alternatively 5′-substitution of a bicyclic nucleic acid (see PCT International Application WO 2007/134181 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., 2007).

In some embodiments, the present disclosure provides oligonucleotides comprising modified nucleosides. Those modified nucleotides may include modified sugars, modified nucleobases, and/or modified linkages. The specific modifications are selected such that the resulting oligonucleotides possess desirable characteristics. In some embodiments, oligonucleotides comprise one or more RNA-like nucleosides. In some embodiments, oligonucleotides comprise one or more DNA-like nucleotides.

In some embodiments, nucleosides of the present disclosure comprise one or more unmodified nucleobases. In certain embodiments, nucleosides of the present disclosure comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (²H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., 1991; and those disclosed by Sanghvi, Y. S., 1993.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, each of which is herein incorporated by reference in its entirety.

In some embodiments, the present disclosure provides oligonucleotides comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or (S), a or R such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present disclosure involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety (Letsinger et al., 1989), cholic acid (Manoharan et al., 1994), a thioether, e.g., hexyl-5-tritylthiol (Manoharan et al., 1992; Manoharan et al., 1993), a thiocholesterol (Oberhauser et al., 1992), an aliphatic chain, e.g., dodecandiol or undecyl residues (Saison-Behmoaras et al., 1991; Kabanov et al., 1990; Svinarchuk et al., 1993), a phospholipid, e.g., di-hexadecyl-rac-glycerol or triethylammonium 1,2-di-O-hexadecyl-rac-glycero-3-H-phosphonate (Manoharan et al., 1995; Shea et al., 1990), a polyamine or a polyethylene glycol chain (Manoharan et al., 1995), or adamantane acetic acid (Manoharan et al., 1995), a palmityl moiety (Mishra et al., 1995), or an octadecylamine or hexylamino-carbonyl-oxycholesterol moiety (Crooke et al., 1996).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

### C. Lipid Nanoparticles

In some aspects of the present disclosure, compositions that contain one or more types of lipid nanoparticles. The lipid nanoparticles may comprise a cationic lipid. In some embodiments, the cationic lipid may be an ionizable lipid. In some embodiments, the cationic lipids are mixed with 1, 2, 3, 4, or 5 different types of lipids. It is contemplated that the cationic lipids can be mixed with multiple different lipids of a single type. In some embodiments, the cationic lipids compositions comprise at least a steroid or a steroid derivative, a PEG lipid, and a phospholipid.

### 1. Cationic Lipids

The present disclosure contemplates the use of lipid nanoparticles. The present lipid nanoparticles may comprise one or more cationic lipids. Preferably, the cationic lipid carries a net positive charge at about physiological pH. The cationic lipid may comprise a permanent positive charge such that it bears that positive charge regardless of the solution pH.

The cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3 dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (D-Lin-DAC 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9412Z)-octadeca-9,12-dienyl)tetrahydro-3aH-cyclopenta[d][1,3]dioxol-5-amine, (649428431Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (649428431Z)-heptatriaconta-6,9,28,31-tetraen-19-yl 4-(dimethylamino) butanoate (DLin-M-C3-DMA), 3-((6Z,9Z,28Z,31Z-heptatriaconta-6,9,28,31-tetraen-19-yloxy)-N,N-dimethylpropan-1-amine (MC3 Ether), 4-((649428431 Z)-heptatriaconta-6,9,28,31-tetraen-19-yloxy)-N,N-dimethylbutan-1-amine (MC4 Ether), or any combination of any of die foregoing. Other cationic lipids include, but are not limited to, N,N-distearyl-N,N-dimethylammonium bromide (DDAB), 3P—(N—(N′,N′-dimethylaminoethane)-carbamoyl)cholesterol (DC-Chol), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC) Additionally, commercial preparations of cationic lipids can be used, such as, e.g., LIPOFECTIN (including DOTMA and DOPE, available from GIBCO/BRL.), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al, PNAS, 107(5), 1864-69, 2010. Other suitable amino lipids include those having alternative fatty acid groups and other dialkylamino groups, including those, in which the alkyl substituents are different (e.g., N-ethyl-N-methylamino- and N-propyl-N-ethylamino-).

In some embodiments, the molar percentage of the cationic lipid relative to the total lipid nanoparticles is from about 10 to about 70. In some embodiments, the molar percentage is 10, 12, 15, 16, 18, 20, 22, 24, 26, 28, 30, 32, 34, 36, 38, 40, 45, 50, 55, 60, 65, 70, or any range derivable therein.

### 2. Steroids and Steroid Derivatives

In some aspects of the present disclosure, the lipid nanoparticles are mixed with one or more steroid or a steroid derivative to create a lipid nanoparticle. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula below:

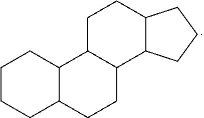 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

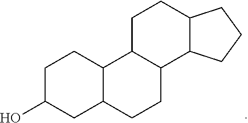 [SMILES: OC1CCC2C(CCC3C4CCCC4CCC23)C1]

In some embodiments of the present disclosure, the steroid or steroid derivative is a cholestane or cholestane derivative. In a cholestane, the ring structure is further defined by the formula:

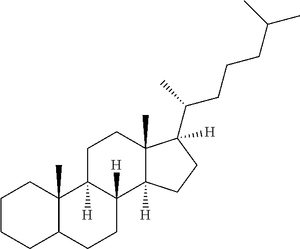

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof. In some embodiments, the compositions comprise cholesterol.

In some embodiments, the compositions may further comprise a molar percentage of the steroid to the total lipid nanoparticles from about 40 to about 46. In some embodiments, the molar percentage is from about 40, 41, 42, 43, 44, 45, to about 46 or any range derivable therein. In other embodiments, the molar percentage of the steroid relative to the total lipid nanoparticles is from about 15 to about 40. In some embodiments, the molar percentage is 15, 16, 18, 20, 22, 24, 26, 28, 30, 32, 34, 36, 38, or 40, or any range derivable therein.

### 3. PEG or PEGylated Lipid

In some aspects of the present disclosure, the lipid nanoparticles comprise one or more PEGylated lipids (or PEG lipid). In some embodiments, the present disclosure comprises using any lipid to which a PEG group has been attached. In some embodiments, the PEG lipid is a diglyceride which also comprises a PEG chain attached to the glycerol group. In other embodiments, the PEG lipid is a compound which contains one or more C6-C24 long chain alkyl or alkenyl group or a C6-C24 fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present disclosure are taught by U.S. Pat. No. 5,820,873, WO 2010/141069, or U.S. Pat. No. 8,450,298, which is incorporated herein by reference.

In another aspect, the PEG lipid has the formula:

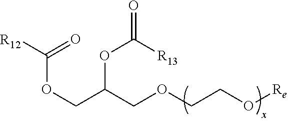

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In another aspect, the PEG lipid has the formula:

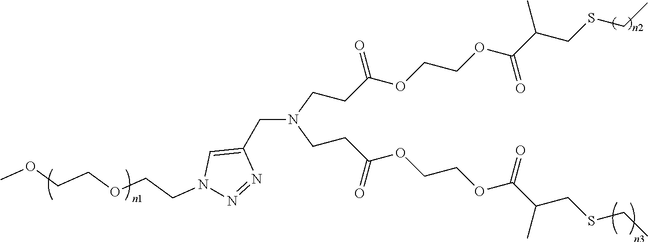

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments, the lipid nanoparticles may further comprise a molar percentage of the PEG lipid to the total lipid nanoparticles from about 4.0 to about 4.6. In some embodiments, the molar percentage is from about 4.0, 4.1, 4.2, 4.3, 4.4, 4.5, to about 4.6 or any range derivable therein. In other embodiments, the molar percentage is from about 1.5 to about 4.0. In some embodiments, the molar percentage is from about 1.5, 1.75, 2, 2.25, 2.5, 2.75, 3, 3.25, 3.5, 3.75, to about 4.0 or any range derivable therein.

### 4. Phospholipid

In some aspects of the present disclosure, the present composition may comprise one or more lipid nanoparticles containing a phospholipid. In some embodiments, any lipid which also comprises a phosphate group. In some embodiments, the phospholipid is a structure which contains one or two long chain C6-C24 alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine.

In some embodiments, the lipid nanoparticles may further comprise a molar percentage of the phospholipid to the total lipid nanoparticles from about 20 to about 23. In some embodiments, the molar percentage is from about 20, 20.5, 21, 21.5, 22, 22.5, to about 23 or any range derivable therein. In other embodiments, the molar percentage is from about 7.5 to about 20. In some embodiments, the molar percentage is from about 7.5, 8, 9, 10, 11, 12, 13, 14, 15, 16, 17, 18, 19, to about 20 or any range derivable therein.

### D. Excipients

In some aspects, the present disclosure comprises one or more excipients formulated into pharmaceutical compositions. The pharmaceutical compositions comprise one or more excipients such as a sugar or sugar alcohol or an amino acid. Furthermore, the compositions may further comprise one or more additional excipients such as a pharmaceutically acceptable polymer. In some embodiments, the weight ratio of the sugar to the amino acid is from about 1:6 to about 20:1, from about 1:2 to about 8:1, or from about 3:2 to about 3:1. In some embodiments, the weight ratio of the sugar to the amino acid is from about 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 to about 20:1, or any range derivable therein. In other embodiments, the pharmaceutical composition does not comprise an amino acid. The pharmaceutical composition may further comprise an amount of either one excipient or a group of excipients from about 20% to about 99.9%, 40% to about 99.5%, or from about 80% to about 99%. The amount of excipients in the pharmaceutical composition may be from about 20%, 25%, 30%, 35%, 40%, 45%, 50%, 55%, 60%, 65%, 70%, 75%, 80%, 82%, 84%, 85%, 85%, 88%, 90%, 92%, 94%, 95%, 96%, 97%, 98%, 99%, 99.2%, 99.4%, 99.5%, 99.6%, 99.8%, or 99.9%, or any range derivable therein.

i. Sugar or Sugar Alcohol

In some aspects, the present disclosure comprises one or more excipients formulated into pharmaceutical compositions. In some embodiments, the excipients used herein are water soluble excipients. These water-soluble excipients include sugars or sugar alcohols such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In other aspects, larger molecules like amino acids, peptides and proteins are incorporated to facilitate inhalation delivery, including leucin, trileucine, histidine and others.

ii. Amino Acids

In some aspects, the present disclosure provides pharmaceutical compositions comprise one or more amino acids, peptides, or proteins. The amino acids may be one of the canonical amino acids such as glycine, alanine, isoleucine, leucine, proline, valine, phenylalanine, tryptophan, tyrosine, aspartic acid, glutamic acid, arginine, histidine, lysine, serine, threonine, cysteine, methionine, asparagine, or glutamine. The amino acids may also be a non-natural amino acid or a modified amino acid such as a glycosylated or phosphorylated amino acid. The amino acids used herein may be in the form of a polypeptide of multiple amino acids or may be a polypeptide of the same amino acids. In particular, polypeptides of 2, 3, 4, 5, 6, 8, 10, 15, 20, or 25 amino acid residues may be used. In other aspects, larger molecules like amino acids, peptides and proteins are incorporated to facilitate inhalation delivery, including leucin, trileucine, histidine and others.

iii. Buffers

In some aspects, the present disclosure provides composition that comprise one or more buffers. The buffers that may be used in the pharmaceutical composition include a phosphate buffer, a succinate buffer, a citrate buffer, a histidine buffer, or an acetate buffer. The buffer may be used may in an aqueous solution. The aqueous solution may further comprise one or more salts such as a saline solution. The buffer may also further comprise one or more organic solvents in trace amounts.

iv. Other Excipients

In some aspects, the present disclosure provides compositions which may further comprise a pharmaceutically acceptable polymer. In some embodiments, the polymer has been approved for use in a pharmaceutical formulation and is known to undergo softening or increased pliability when raised above a specific temperature without substantially degrading. It is also contemplated that the present compositions may comprise one or more mucoadhesive polymers. Some non-limiting examples of mucoadhesive polymers include lectins, fimbrin, sodium alginate, sodium carboxymethylcellulose, guar gum, hydroxyethylcellulose, karya gum, methylcellulose, poly(ethylene glycol) (PEG), retene, polyacrylate, starch, chitosan, gellan, or tragacanth.

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups which are charged at a physiologically relevant pH. Some non-limiting examples of neutral non-cellulosic polymers include polyvinyl pyrrolidone, polyvinyl alcohol, copovidone, and poloxamer. Within this class, in some embodiments, pyrrolidone containing polymers are particularly useful. Some non-limiting examples of ionizable cellulosic polymers include cellulose acetate phthalate and hydroxypropyl methyl cellulose acetate succinate. Finally, some non-limiting examples of neutral cellulosic polymers include hydroxypropyl cellulose, hydroxypropyl methyl cellulose, hydroxyethyl cellulose, and hydroxymethyl cellulose.

Some specific pharmaceutically acceptable polymers which may be used include, for example, Eudragit™ RS PO, Eudragit™ S100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate-methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate-co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate-co-methyl methacrylate-co-trimethylammonioethyl methacrylate chloride 1:2:0.1), Eudragit L-30-D™ (MA-EA, 1:1), Eudragit L-100-55™ (MA-EA, 1:1), hydroxypropylmethylcellulose acetate succinate (HPMCAS), polyvinyl caprolactam-polyvinyl acetate-PEG graft copolymer, polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, and xanthan gum.

In some embodiments, the compositions described herein contain a pharmaceutically acceptable polymer selected from povidone, copovidone, polyvinyl pyrrolidone, polyvinyl acetate, and SOLUPLUS® (polyvinyl caprolactampolyvinyl acetate-polyethylene glycol graft co-polymer, commercially available from BASF). In particular, the pharmaceutical acceptable polymer may be a copolymer of polyvinyl pyrrolidone and polyvinyl acetate. In particular, the copolymer may comprise about 5-7 vinyl pyrrolidone units to about 3-5 units of vinyl acetate, in particular 6 units of vinyl pyrrolidone and 4 units of vinyl acetate. The number-average of the molecular weight of the polymer may be from about 15,000 to about 20,000 Dalton. The pharmaceutically acceptable polymer may be Kollidan® VA 64 (copovidone, vinylpyrrolidone-vinyl acetate) having a CAS Number of 25086-89-9.

In some embodiments, the excipient used herein is a pharmaceutically acceptable polymer, such as chitosan, alginate, gellan, starch, polyacrylate, polyvinylpyrrolidine, or cellulose. In some embodiments, the excipient is polyvinylpyrrolidone. In some embodiments, the excipient is polyvinylpyrrolidone with a molecular weight from about 10,000 Daltons to about 80,000 Daltons, or from about 10,000 Daltons, 20,000 Daltons, 30,000 Daltons, 40,000 Daltons, 50,000 Daltons, 60,000 Daltons, 70,000 Daltons, to about 80,000 Daltons, or any range derivable therein.

In some embodiments, one or more surface modifying excipients may be added. These surface modifying excipients include poloxomers and tweens, such as Poloxomer 188 and Tween 20.

In some aspects, the amount of the excipient in the pharmaceutical composition is from about 0.5% to about 20% w/w, from about 1% to about 10% w/w, from about 2% to about 8% w/w, or from about 2% to about 5% w/w. The amount of the excipient in the precursor solution comprises from about 0.5%, 0.75%, 1%, 1.25%, 1.5%, 1.75%, 2%, 2.5%, 3%, 3.5%, 4%, 4.5%, 5%, 6%, 7%, 8%, 9%, to about 10% w/w, or any range derivable therein.

### IV. Methods

In an aspect is provided a method for preparing a thin film including applying a liquid liposomal formulation to a freezing surface; allowing the liquid formulation to spread and freeze on the freezing surface thereby forming a thin film.

In certain aspects, the present disclosure provides pharmaceutical compositions which may be prepared using a URF process, such as thin-film freezing process. Such methods are described in U.S. Patent Application No. 2010/0221343 and Watts, et al., 2013, both of which are incorporated herein by reference. In some cases, the methods employ an ultra-rapid freezing rate of up to 10,000 K/sec, e.g., at least 100, 200, 500, 800, 1,000, 2,000, 5,000 or 8,000 K/sec. In some embodiments, these methods involve dissolving or dispersing the components of the pharmaceutical composition into a solvent to form a pharmaceutical mixture. The solvents may be either water or an organic solvent or a mixture of water and organic solvent. In some embodiments, the solvent is water. In some embodiments, the solvent is saline. In some embodiments, the solvent is phosphate buffered saline. In other embodiments, the solvent is citrate buffer, histidine buffer, or succinate buffer.

In some embodiments, an amino acid is further dissolved in the pharmaceutical mixture. In some embodiments, the amino acid is a canonical amino acid. In some embodiments, the amino acid is a non-polar amino acid, such as leucine. In some embodiments, the one or more therapeutic agents, sugar or sugar alcohol, and amino acid are dissolved at a dissolving temperature. In some embodiments, the dissolving temperature is from about −10° C. to about 40° C., from about −5° C. to about 25° C., from about 0° C. to about 10° C., or from about −10° C., −5° C., 0° C., 5° C., 10° C., 15° C., 20° C., 25° C., 30° C., 35° C., to about 40° C., or any range derivable therein. In some embodiments, the pharmaceutical mixture is admixed until the pharmaceutical mixture is clear.

In some embodiments, the pharmaceutical mixture is an aqueous solution that includes one or more therapeutic agent with one or more excipients. In some embodiments, the pharmaceutical mixture is an aqueous solution or dispersion that includes a nucleic acid-based product and a sugar or sugar alcohol. In some embodiments, the pharmaceutical mixture may contain a solid content from about 0.05% w/v to about 5% w/v, from about 0.1% w/v to about 2.5% w/v, from about 1.0% w/v to about 3.0% w/v, from about 0.15% w/v to about 1.5% w/v, from about 0.2% w/v to about 0.6% w/v, or from about 0.5% w/v to about 1.25% w/v of the pharmaceutical composition, or from about 2.50 w/v, 2.75 w/v, 3.00 w/v, 3.25 w/v, 3.50 w/v, 3.75 w/v, 4.00 w/v, 4.50 w/v, 5.00 w/v, 5.50 w/v, 6.00 w/v, 6.50 w/v, 7.00 w/v, 7.50 w/v, 8.00 w/v, 9.00 w/v, 10.00 w/v, 11.00 w/v, 12.00 w/v, 13.00 w/v, 14.00 w/v, 15.00 w/v, 16.00 w/v, 17.00 w/v, 18.00 w/v, 19.00 w/v, to about 20.00 w/v, or any range derivable therein.

This precursor solution may be deposited on a surface which is at a temperature that causes the pharmaceutical mixture to freeze. In some embodiments, this temperature may be below the freezing point of the solution at ambient pressure. In other embodiments, a reduced pressure may be applied to the surface causing the solution to freeze at a temperature below the ambient pressure's freezing point. In some embodiments, the surface temperature is below 0° C. The surface may also be rotating or moving on a moving conveyer-type system thus allowing the precursor solution to distribute evenly on the surface. Alternatively, the precursor solution may be applied to surface in such a manner to generate an even surface.

After the precursor solution has been applied to the surface, the solvent may be removed to obtain a pharmaceutical composition. Any appropriate method of removing the solvent may be applied including evaporation under reduced pressure or elevated temperature or lyophilization. In some embodiments, the lyophilization may comprise a first reduced pressure and/or a first reduced temperature. Such a first reduced temperature may be from 0° C. to about −100° C., from −20° C. to about −60° C., or from about 0° C., −10° C., −20° C., −30° C., −40° C., −50° C., −60° C., −70° C., −80° C., −90° C., to about −100° C., or any range derivable therein. Additionally, the solvent may be removed at a first reduced pressure of from about 10 mTorr to 500 mTorr, from about 50 mTorr to about 250 mTorr, or from about 10 mTorr, 20 mTorr, 30 mTorr, 40 mTorr, 50 mTorr, 100 mTorr, 150 mTorr, 200 mTorr, 250 mTorr, 300 mTorr, 400 mTorr, to about 500 mTorr, or any range derivable therein. In some embodiments, the frozen pharmaceutical composition is dried for a primary drying time period from about 3 hours to about 2 weeks, from about 6 hours to about 1 week, or from about 3 hours, 4 hours, 5 hours, 6 hours, 12 hours, 18 hours, 24 hours, 30 hours 36 hours, 2 days, 3 days, 4 days, 5 days, 6 days, 1 week, 1.5 weeks, 2 weeks, to about 3 weeks, or any range derivable therein.

In some embodiments, the frozen pharmaceutical composition is dried in a secondary drying time period. In some embodiments, the frozen pharmaceutical composition is dried a secondary drying time at a second reduced pressure. In some embodiments, the secondary drying time is at a reduced pressure is from about 10 mTorr to 500 mTorr, from about 50 mTorr to about 250 mTorr, or from about 10 mTorr to 500 mTorr, from about 50 mTorr to about 250 mTorr, or from about 10 mTorr, 20 mTorr, 30 mTorr, 40 mTorr, 50 mTorr, 100 mTorr, 150 mTorr, 200 mTorr, 250 mTorr, 300 mTorr, 400 mTorr, to about 500 mTorr, or any range derivable therein. In some embodiments, the frozen pharmaceutical composition is dried for a secondary drying time at a second reduced temperature. In some embodiments, the second reduced temperature is from about 0° C. to 30° C., from about 10° C. to about 30° C., or from about 0° C., 5° C., 10° C., 15° C., 20° C., 25° C., to about 30° C., or any range derivable therein. In some embodiments, the frozen pharmaceutical composition is dried for a second time period from about 3 hours to about 2 weeks, from about 6 hours to about 1 week, or from about 3 hours, 4 hours, 5 hours, 6 hours, 12 hours, 18 hours, 24 hours, 30 hours 36 hours, 2 days, 3 days, 4 days, 5 days, 6 days, 1 week, 1.5 weeks, 2 weeks, to about 3 weeks, or any range derivable therein. In some embodiments, the temperature is changed from the first reduced temperature to the second reduced temperature over a ramping time period. In some embodiments, the ramping time period is from about 3 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein.

In some embodiments, the pharmaceutical composition has a water content of less than 10%, less than 7.5%, or less than 5%, or any range derivable therein.

Such as composition prepared using these methods may exhibit a brittle nature such that the composition is easily sheared into smaller particles when processed through a device. These compositions have high surface areas as well as exhibit improved flowability of the composition. Such flowability may be measured, for example, by the Carr index or other similar measurements. In particular, the Carr index may be measured by comparing the bulk density of the powder with the tapped density of the powder. Such composition may exhibit a favorable Carr index and may result in the particles being better sheared to give smaller particles when the composition is processed through a secondary device to further process a powder composition. Such composition may exhibit a favorable Carr's index after it is processed. In some embodiments, the composition may be processed by micronization such as vortexing or ball milling.

In embodiments, the liquid formulation includes an excipient. In embodiments, the liquid formulation includes a plurality of different excipients. In embodiments, the excipient is a salt, buffer, detergent, polymer, amino acid, or preservative. In embodiments, the excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benzyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. In embodiments, the excipient is trehalose. In embodiments, the liquid formulation includes less than 5% wt/vol of the excipient/liquid formulation. In embodiments, the liquid formulation includes less than 4% wt/vol of the excipient/liquid formulation. In embodiments, the liquid formulation includes less than 3% wt/vol of the excipient/liquid formulation. In embodiments, the liquid formulation includes less than 2% wt/vol of the excipient/liquid formulation. In embodiments, the liquid formulation includes less than 1% wt/vol of the excipient/liquid formulation. In embodiments, the liquid formulation includes less than 0.5% wt/vol of the excipient/liquid formulation. In embodiments, the liquid formulation includes about 5% wt/vol of the excipient/liquid formulation. In embodiments, the liquid formulation includes about 4% wt/vol of the excipient/liquid formulation. In embodiments, the liquid formulation includes about 3% wt/vol of the excipient/liquid formulation. In embodiments, the liquid formulation includes about 2% wt/vol of the excipient/liquid formulation. In embodiments, the liquid formulation includes about 1% wt/vol of the excipient/liquid formulation. In embodiments, the liquid formulation includes about 0.5% wt/vol of the excipient/liquid formulation. In embodiments, the liquid formulation includes about 0.1, 0.2, 0.3, 0.4, 0.5, 0.6, 0.7, 0.8, 0.9, 1, 2, 3, 4, 5, 6, 7, 8, 9, or 10% (wt/vol) of the excipient/liquid formulation. In embodiments, the liquid formulation includes 0.1, 0.2, 0.3, 0.4, 0.5, 0.6, 0.7, 0.8, 0.9, 1, 2, 3, 4, 5, 6, 7, 8, 9, or 10% (wt/vol) of the excipient/liquid formulation. In embodiments, the liquid formulation includes less than 5% of the excipient. In embodiments, the liquid formulation includes less than 4% of the excipient. In embodiments, the liquid formulation includes less than 3% of the excipient. In embodiments, the liquid formulation includes less than 2% of the excipient. In embodiments, the liquid formulation includes less than 1% of the excipient. In embodiments, the liquid formulation includes less than 0.5% of the excipient. In embodiments, the liquid formulation includes about 5% of the excipient. In embodiments, the liquid formulation includes about 4% of the excipient. In embodiments, the liquid formulation includes about 3% of the excipient. In embodiments, the liquid formulation includes about 2% of the excipient. In embodiments, the liquid formulation includes about 1% of the excipient. In embodiments, the liquid formulation includes about 0.5% of the excipient. In embodiments, the liquid formulation includes about 0.1, 0.2, 0.3, 0.4, 0.5, 0.6, 0.7, 0.8, 0.9, 1, 2, 3, 4, 5, 6, 7, 8, 9, or 10% of the excipient. In embodiments, the liquid formulation includes 0.1, 0.2, 0.3, 0.4, 0.5, 0.6, 0.7, 0.8, 0.9, 1, 2, 3, 4, 5, 6, 7, 8, 9, or 10% of the excipient.

In embodiments, the applying includes spraying or dripping droplets of the liquid formulation. In embodiments, the vapor-liquid interface of the droplets is less than 500 cm⁻¹ area/volume. In embodiments, the vapor-liquid interface of the droplets is less than 400 cm⁻¹ area/volume. In embodiments, the vapor-liquid interface of the droplets is less than 300 cm⁻¹ area/volume. In embodiments, the vapor-liquid interface of the droplets is less than 200 cm⁻¹ area/volume. In embodiments, the vapor-liquid interface of the droplets is less than 100 cm⁻¹ area/volume. In embodiments, the vapor-liquid interface of the droplets is less than 50 cm⁻¹ area/volume. In embodiments, the vapor-liquid interface of the droplets is less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹ area/volume.

In embodiments, the method further includes contacting the droplets with a freezing surface having a temperature below the freezing temperature of the liquid formulation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C. below the freezing temperature). In embodiments, the method further includes contacting the droplets with a freezing surface having a temperature differential of at least 30° C. between the droplets and the surface. In embodiments, the temperature differential is at least 40° C. between the droplets and the surface. In embodiments, the temperature differential is at least 50° C. between the droplets and the surface. In embodiments, the temperature differential is at least 60° C. between the droplets and the surface. In embodiments, the temperature differential is at least 70° C. between the droplets and the surface. In embodiments, the temperature differential is at least 80° C. between the droplets and the surface. In embodiments, the temperature differential is at least 90° C. between the droplets and the surface. In embodiments, the temperature differential between the droplets and the surface is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 180 or 200° C.

In embodiments, the thin film has a thickness of about 5 mm, about 4 mm, about 3 mm, about 2 mm, about 1 mm, less than 3 mm, less than 2 mm, less than 1 mm, less than 500 micrometers. In embodiments, the thin film has a thickness of less than 400 micrometers. In embodiments, the thin film has a thickness of less than 300 micrometers. In embodiments, the thin film has a thickness of less than 200 micrometers. In embodiments, the thin film has a thickness of less than 100 micrometers. In embodiments, the thin film has a thickness of less than 50 micrometers. In embodiments, the thin film has a thickness of less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers. In embodiments, the thin film has a thickness of about 500 micrometers. In embodiments, the thin film has a thickness of about 400 micrometers. In embodiments, the thin film has a thickness of about 300 micrometers. In embodiments, the thin film has a thickness of about 200 micrometers. In embodiments, the thin film has a thickness of about 100 micrometers. In embodiments, the thin film has a thickness of about 50 micrometers. In embodiments, the thin film has a thickness of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers.

In embodiments, the thin film has a surface area to volume ratio of between about 5 and 500 cm⁻¹. In embodiments, the thin film has a surface area to volume ratio of between 25 and 400 cm⁻¹. In embodiments, the thin film has a surface area to volume ratio of between 25 and 300 cm⁻¹. In embodiments, the thin film has a surface area to volume ratio of between 25 and 200 cm⁻¹. In embodiments, the thin film has a surface area to volume ratio of between 25 and 100 cm⁻¹. In embodiments, the thin film has a surface area to volume ratio of between 100 and 500 cm⁻¹. In embodiments, the thin film has a surface area to volume ratio of between 200 and 500 cm⁻¹. In embodiments, the thin film has a surface area to volume ratio of between 300 and 500 cm⁻¹. In embodiments, the thin film has a surface area to volume ratio of between 400 and 500 cm⁻¹. In embodiments, the thin film has a surface area to volume ratio of between 100 and 400 cm⁻¹. In embodiments, the thin film has a surface area to volume ratio of between 200 and 300 cm⁻¹. In embodiments, the thin film has a surface area to volume ratio of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹. In embodiments, the thin film has a surface area to volume ratio of between about 25 and about 500 cm⁻¹. In embodiments, the thin film has a surface area to volume ratio of between about 25 and about 400 cm⁻¹. In embodiments, the thin film has a surface area to volume ratio of between about 25 and about 300 cm⁻¹. In embodiments, the thin film has a surface area to volume ratio of between about 25 and about 200 cm⁻¹. In embodiments, the thin film has a surface area to volume ratio of between about 25 and about 100 cm⁻¹. In embodiments, the thin film has a surface area to volume ratio of between about 100 and about 500 cm⁻¹. In embodiments, the thin film has a surface area to volume ratio of between about 200 and about 500 cm⁻¹. In embodiments, the thin film has a surface area to volume ratio of between about 300 and about 500 cm⁻¹. In embodiments, the thin film has a surface area to volume ratio of between about 400 and about 500 cm⁻¹. In embodiments, the thin film has a surface area to volume ratio of between about 100 and about 400 cm⁻¹. In embodiments, the thin film has a surface area to volume ratio of between about 200 and about 300 cm⁻¹.

In embodiments, the freezing rate of the droplets is between about 10 K/second and about 10³ K/second. In embodiments, the freezing rate of the droplets is between about 10² K/second and about 10³ K/second. In embodiments, the freezing rate of the droplets is between about 50 K/second and about 5×10² K/second. In embodiments, the freezing rate of the droplets is between 10 K/second and 10⁵ K/second. In embodiments, the freezing rate of the droplets is between 10 K/second and 10⁴ K/second. In embodiments, the freezing rate of the droplets is between 10 K/second and 10³ K/second. In embodiments, the freezing rate of the droplets is between 10² K/second and 10³ K/second. In embodiments, the freezing rate of the droplets is between 50 K/second and 5×10² K/second. In embodiments, the freezing rate of the droplets is about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, the freezing rate of the droplets is 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, each of the droplets freezes upon contact with the freezing surface in less than about 50, 75, 100, 125, 150, 175, 200, 250, 500, 1,000, or 2,000 milliseconds. In embodiments, each of the droplets freezes upon contact with the freezing surface in less than 50, 75, 100, 125, 150, 175, 200, 250, 500, 1,000, or 2,000 milliseconds.

In embodiments, the droplets have an average diameter between about 0.1 and about 5 mm at a temperature between about 2 and about 25° C. In embodiments, the droplets have an average diameter between about 2 and about 4 mm at a temperature between about 20 and about 25° C. In embodiments, the droplets have an average diameter between about 1 and about 4 mm at a temperature between about 2 and about 25° C. In embodiments, the droplets have an average diameter between about 2 and about 3 mm at a temperature between about 2 and about 25° C. In embodiments, the droplets have an average diameter between about 1 and about 3 mm at a temperature between about 2 and about 25° C. In embodiments, the droplets have an average diameter between about 1 and about 2 mm at a temperature between about 2 and about 25° C. In embodiments, the droplets have an average diameter between about 3 and about 4 mm at a temperature between about 2 and about 25° C. In embodiments, the droplets have an average diameter between 0.1 and 5 mm at a temperature between 2 and 25° C. In embodiments, the droplets have an average diameter between 2 and 4 mm at a temperature between 2 and 25° C. In embodiments, the droplets have an average diameter between 1 and 4 mm at a temperature between 2 and 25° C. In embodiments, the droplets have an average diameter between 2 and 3 mm at a temperature between 2 and 25° C. In embodiments, the droplets have an average diameter between 1 and 3 mm at a temperature between 2 and 25° C. In embodiments, the droplets have an average diameter between 1 and 2 mm at a temperature between 2 and 25° C. In embodiments, the droplets have an average diameter between 3 and 4 mm at a temperature between 2° and 25° C.

In embodiments, the method further includes removing the solvent (e.g., water or liquid) from the thin film to form a dry liposomal formulation.

In embodiments, is a method of making a dry formulation from a thin film (e.g., including a thin film made using a method as described herein), including removing the solvent (e.g., water or liquid) from the thin film to form a dry formulation. In embodiments of the methods described herein, the dry formulation is a dry formulation as described herein, including in an aspect, embodiment, example, table, figure, or claim. In embodiments, a method of making a thin film or a method of making dry formulation is used to make a dry formulation as described herein, including in an aspect, embodiment, example, table, figure, or claim.

In embodiments, the removing of the solvent includes lyophilization. In embodiments, the removing of the solvent includes lyophilization at temperatures of −20° C. or less. In embodiments, the removing of the solvent includes lyophilization at temperatures of −25° C. or less. In embodiments, the solvent includes lyophilization at temperatures of −40° C. or less. In embodiments, the removing of the solvent includes lyophilization at temperatures of −50° C. or less. In embodiments, the removing of the solvent includes lyophilization at temperatures of about −20° C. or less. In embodiments, the removing of the solvent includes lyophilization at temperatures of about −25° C. or less. In embodiments, the solvent includes lyophilization at temperatures of about −40° C. or less. In embodiments, the removing of the solvent includes lyophilization at temperatures of about −50° C. or less. Primary drying can be performed at −20° C. to −50° C., and secondary drying can be performed at 4-25° C.

In embodiments, the reconstituted liquid formulation includes particles of the same average diameter as the liquid formulation (prior to forming the dry formulation from the liquid formulation) particles. In embodiments, the reconstituted liquid formulation includes particles having an average diameter within 5% of the average diameter of particles in the liquid formulation (prior to forming the dry formulation from the liquid formulation). In embodiments, the reconstituted liquid formulation includes particles having an average diameter within 10% of the average diameter of particles in the liquid formulation (prior to forming the dry formulation from the liquid formulation). In embodiments, the reconstituted liquid formulation includes particles having an average diameter within 20% of the average diameter of particles in the liquid formulation (prior to forming the dry formulation from the liquid formulation). In embodiments, the reconstituted liquid formulation includes particles having an average diameter within 10%, 20%, 30% or 40% of the average diameter of particles in the liquid formulation (prior to forming the dry formulation from the liquid formulation). There may less than 5% or 10% of the particles aggregated (e.g., 8 particles can aggregate together to form a single particle with a diameter approximately twice that of the original smaller particles). In embodiments, the reconstituted liquid formulation includes particles having an average diameter within about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% of the average diameter of particles in the liquid formulation (prior to forming the dry formulation from the liquid formulation). In embodiments, the reconstituted liquid formulation includes particles having an average diameter within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% of the average diameter of particles in the liquid formulation (prior to forming the dry formulation from the liquid formulation).

In embodiments, the solvating, reconstituting or rehydrating of the dry formulation is at least one day after preparing the dry formulation from the liquid formulation (e.g., the dry formulation is stored for at least one day). In embodiments, the solvating of the dry formulation is at least two days after preparing the dry formulation from the liquid formulation (e.g., the dry formulation is stored for at least two days). In embodiments, the solvating of the dry formulation is at least three days after preparing the dry formulation from the liquid formulation (e.g., the dry formulation is stored for at least three days). In embodiments, the solvating of the dry formulation is at least one week after preparing the dry formulation from the liquid formulation (e.g., the dry formulation is stored for at least one week). In embodiments, the solvating of the dry formulation is at least two weeks after preparing the dry formulation from the liquid formulation (e.g., the dry formulation is stored for at least two weeks). In embodiments, the solvating of the dry formulation is at least one month after preparing the dry formulation from the liquid formulation (e.g., the dry formulation is stored for at least one month). In embodiments, the solvating of the dry formulation is at least two months after preparing the dry formulation from the liquid formulation (e.g., the dry formulation is stored for at least two months). In embodiments, the solvating of the dry formulation is at least three months after preparing the dry formulation from the liquid formulation (e.g., the dry formulation is stored for at least three months). In embodiments, the solvating of the dry formulation is at least six months after preparing the dry formulation from the liquid formulation (e.g., the dry formulation is stored for at least six months). In embodiments, the solvating of the dry formulation is at least one year after preparing the dry formulation from the liquid formulation (e.g., the dry formulation is stored for at least one year). In embodiments, the solvating of the dry formulation is at least two years after preparing the dry formulation from the liquid formulation (e.g., the dry formulation is stored for at least two years). In embodiments, the solvating of the dry formulation is at least three years after preparing the dry formulation from the liquid formulation (e.g., the dry formulation is stored for at least three years). In embodiments, the solvating of the dry formulation is at least five years after preparing the dry formulation from the liquid formulation (e.g., the dry formulation is stored for at least five years). In embodiments, the solvating of the dry formulation is at least ten years after preparing the dry formulation from the liquid formulation (e.g., the dry formulation is stored for at least ten years).

In embodiments, prior to the solvating, reconstituting or rehydrating of the dry formulation, the dry formulation is stored at about 4° C. for at least 99% of the time. In embodiments, prior to the solvating of the dry formulation, the dry formulation is stored at less than 4° C. for at least 99% of the time. In embodiments, prior to the solvating of the dry formulation, the dry formulation is stored at less than 0° C. for at least 99% of the time. In embodiments, prior to the solvating of the dry formulation, the dry formulation is stored at less than −20° C. for at least 99% of the time. In embodiments, prior to the solvating of the dry formulation, the dry formulation is stored at about −20° C. for at least 99% of the time. In embodiments, prior to the solvating of the dry formulation, the dry formulation is stored at less than −80° C. for at least 99% of the time. In embodiments, prior to the solvating of the dry formulation, the dry formulation is stored at about −80° C. for at least 99% of the time. In embodiments, prior to the solvating of the dry formulation, the dry formulation is stored at ambient temperatures (e.g., room temperature). In embodiments, prior to the solvating of the dry formulation, the dry formulation is stored at between 2° and 24° C. for at least 99% of the time. In embodiments, prior to the solvating of the dry formulation, the dry formulation is stored at between 4 and 24° C. for at least 99% of the time. In embodiments, prior to the solvating of the dry formulation, the dry formulation is stored at between 0 and 24° C. for at least 99% of the time. In embodiments, prior to the solvating of the dry formulation, the dry formulation is stored at between 4 and 40° C. for at least 99% of the time. In embodiments, prior to the solvating of the dry formulation, the dry formulation is stored at between 0 and 40° C. for at least 99% of the time. In embodiments, prior to the solvating of the dry formulation, the dry formulation is stored at about 4° C. for at least 90% of the time. In embodiments, prior to the solvating of the dry formulation, the dry formulation is stored at less than 4° C. for at least 90% of the time. In embodiments, prior to the solvating of the dry formulation, the dry formulation is stored at less than 0° C. for at least 90% of the time. In embodiments, prior to the solvating of the dry formulation, the dry formulation is stored at less than −20° C. for at least 90% of the time. In embodiments, prior to the solvating of the dry formulation, the dry formulation is stored at between 2° and 24° C. for at least 90% of the time. In embodiments, prior to the solvating of the dry formulation, the dry formulation is stored at between 4 and 24° C. for at least 90% of the time. In embodiments, prior to the solvating of the dry formulation, the dry formulation is stored at between 0 and 24° C. for at least 90% of the time. In embodiments, prior to the solvating of the dry formulation, the dry formulation is stored at between 4 and 40° C. for at least 90% of the time. In embodiments, prior to the solvating of the dry formulation, the dry formulation is stored at between 0 and 40° C. for at least 90% of the time.

In an aspect is provided a method of treating a disease in a patient in need of such treatment, the method including administering a therapeutically effective amount of a solvated dry formulation as described herein to the patient.

In an aspect is provided a method of treating a disease in a patient in need of such treatment, the method including administering a therapeutically effective amount of dry formulations described herein (e.g., in an aspect, embodiment, example, table, figure, or claims).

In embodiments, the dry formulation is administered by inhalation, intradermally, orally, or vaginally. In embodiments, the dry formulation is administered through the nasal mucosa.

In embodiments, the method is a method described herein, including in an aspect, embodiment, example, table, figure, or claim. Provided herein is a method of preparing a dry formulation including a method of preparing a thin film as described herein (including in an aspect, embodiment, example, table, figure, or claim) and a method of removing a solvent from a thin film as described herein (including in an aspect, embodiment, example, table, figure, or claim). Provided herein is a method of preparing a dry formulation including a method of preparing a dry formulation as described herein (including in an aspect, embodiment, example, table, figure, or claim), a method of preparing a thin film as described herein (including in an aspect, embodiment, example, table, figure, or claim) and a method of removing a solvent from a thin film as described herein (including in an aspect, embodiment, example, table, figure, or claim).

In embodiments, to form a powder, an aqueous liposomal composition is first frozen to form a frozen composition, then the frozen water is removed to form the powder. A fast-freezing process is used to form the frozen composition. A fast-freezing process, as used herein, is a process that can freeze a thin film of liquid (less than about 500 microns or 2-4 mm) in a time of less than or equal to about 3000 milliseconds. In the TFF process liquid droplets fall from a given height and impact, spread, and freeze on a cooled solid substrate. Typically, the substrate is a metal drum that is cooled to below 250° K, or below 200° K or below 150° K. On impact the droplets that are deformed into thin films freeze in a time of between about 70 ms and 3000 ms. The frozen thin films may be removed from the substrate by a stainless-steel blade mounted along the rotating drum surface. The frozen thin films are collected in liquid nitrogen to maintain in the frozen state. Further details regarding thin film freezing processes may be found in the paper to Engstrom et al. “Formation of Stable Submicron Protein Particles by Thin Film Freezing” Pharmaceutical Research, Vol. 25, No. 6, June 2008, 1334-1346, which is incorporated herein by reference.

Water (e.g., frozen water) is removed from the frozen composition to produce a dry powder. Water (e.g., frozen water) may be removed by a lyophilization process or a freeze-drying process. Water may also be removed by an atmospheric freeze-drying process.

The resulting powder can be readily reconstituted to form a stable dispersion without significant loss of stability or activity. The powder may be transported and stored in a wide range of temperatures without concern of accidental exposure to freezing conditions. In fact, it is generally less costly to transport dry solid powder than liquid.

Described herein are compositions and methods for preparing a thin film or a dry formulation by spraying or dripping droplets of a liquid formulation such that the formulation is exposed to an vapor-liquid interface of less than 500 cm⁻¹ area/volume, such as 25 to 500 cm⁻¹ (e.g., less than 50, 100, 150, 200, 250, 300, 400) and contacting the droplet with a freezing surface having a temperature lower than the freezing temperature of the liquid formulation (e.g., has a temperature differential of at least 30° C. between the droplet and the surface), wherein the surface freezes the droplet into a thin film with a thickness of less than 5 mm, such as about 2-4 mm, about 1 mm, about 500 micrometers (e.g., 450, 400, 350, 300, 250, 200, 150, 100, or 50 micrometers). In embodiments, the method may further include the step of removing the liquid (e.g., solvent, water) from the frozen material to form a dry formulation. In embodiments, the droplets freeze upon contact with the surface in less than 50, 75, 100, 125, 150, 175, 200, 250, 500, 1,000, 2,000, or 3000 milliseconds. In embodiments, the droplets freeze upon contact with the surface in less than 50 or 150 milliseconds. In embodiments, the droplet has a diameter between 2 and 5 mm at room temperature. In embodiments, the droplet forms a thin film on the freezing surface of between 50 micrometers and 5 mm, such as 2-4 mm in thickness. In embodiments, the droplets have a cooling rate of between 50-250 K/s. In embodiments, the particles of the dry formulation, after liquid (e.g., solvent or water) removal, have a surface area of at least 10, 15, 25, 50, 75, 100, 125, 150 or 200 m²/gr (e.g., surface area of 10, 15, 25, 50, 75, 100, 125, 150 or 200 m²/gr).

In embodiments, the droplets may be delivered to the cold or freezing surface in a variety of manners and configurations. In embodiments, the droplets may be delivered in parallel, in series, at the center, middle or periphery or a platen, platter, plate, roller, conveyor surface. In embodiments, the freezing or cold surface may be a roller, a belt, a solid surface, circular, cylindrical, conical, oval and the like that permit for the droplet to freeze. For a continuous process a belt, platen, plate or roller may be particularly useful. In embodiments, the frozen droplets may form beads, strings, films or lines of frozen liquid formulation. In embodiments, the effective ingredient is removed from the surface with a scraper, wire, ultrasound or other mechanical separator prior to the lyophilization process. Once the material is removed from the surface of the belt, platen, roller or plate the surface is free to receive additional material.

In embodiments, the surface is cooled by a cryogenic solid, a cryogenic gas, a cryogenic liquid or a heat transfer fluid capable of reaching cryogenic temperatures or temperatures below the freezing point of the liquid formulation (e.g., at least 30° C. less than the temperature of the droplet). In embodiments, the liquid formulation further includes one or more excipients selected from surfactants, polymeric surfactants, vesicles, polymers, including copolymers and homopolymers and biopolymers, dispersion aids, and serum albumin. In embodiments, the temperature differential between the droplet and the surface is at least 30° C. In embodiments, the excipients or stabilizers that can be included in the liquid formulations that are to be frozen as described herein include: cryoprotectants, lyoprotectants, surfactants, fillers, stabilizers, polymers, protease inhibitors, antioxidants and absorption enhancers. Specific nonlimiting examples of excipients that may be included in the formulations described herein include: sucrose, trehalose, Span 80, Tween 20, Brij 35, Brij 98, Pluronic, sucroester 7, sucroester 11, sucroester 15, sodium lauryl sulfate, oleic acid, laureth-9, laureth-8, lauric acid, vitamin E TPGS, Gelucire 50/13, Gelucire 53/10, Labrafil, dipalmitoyl phosphadityl choline, glycolic acid and salts, deoxycholic acid and salts, sodium fusidate, cyclodextrins, polyethylene glycols, labrasol, polyvinyl alcohols, polyvinyl pyrrolidones and tyloxapol.

In embodiments, the method may further include the step of removing the liquid (e.g., solvent or water) from the frozen liquid formulation to form a dry formulation. In embodiments, the solvent further includes at least one or more excipient or stabilizers selected from, e.g., surfactants, polymeric surfactants, vesicles, polymers, including copolymers and homopolymers and biopolymers, dispersion aids, and serum albumin. In embodiments, the temperature differential between the solvent and the surface is at least about 30° C.

In embodiments, is described a single-step, single-vial method for preparing a thin film or dry formulation by reducing the temperature of a vial wherein the vial has a temperature below the freezing temperature of a liquid formulation (e.g., a temperature differential of at least 30° C. between the liquid formulation and the vial) and spraying or dripping droplets of a liquid formulation directly into the vial such that the liquid formulation is exposed to a vapor-liquid interface of less than 500 cm⁻¹ area/volume, wherein the surface freezes the droplet into a thin film with a thickness of less than 500 micrometers and a surface area to volume between 25 to 500 cm⁻¹. In embodiments, the droplets freeze upon contact with the surface in less than about 50, 75, 100, 125, 150, 175, 200, 250, 500, 1,000, 2,000 or 3000 milliseconds (e.g., in about 50, 75, 100, 125, 150, 175, 200, 250, 500, 1,000 or 2,000 or 3000 milliseconds), and may freeze upon contact with the surface in about 50 or 150 to 500 milliseconds. In embodiments, a droplet has a diameter between 0.1 and 5 mm at room temperature (e.g., a diameter between 2 and 4 mm at room temperature). In embodiments, the droplet forms a thin film on the surface of between 50 micrometers to 5 mm, such as about 2-4 millimeters in thickness. In embodiments, the droplets have a cooling rate of between 50-250 K/s. In embodiments, the vial may be cooled by a cryogenic solid, a cryogenic gas, a cryogenic liquid, a freezing fluid, a freezing gas, a freezing solid, a heat exchanger, or a heat transfer fluid capable of reaching cryogenic temperatures or temperatures below the freezing point of the liquid formulation. In embodiments, the vial may be rotated as the spraying or droplets are delivered to permit the layering or one or more layers of the liquid formulation. In embodiments, the vial and the liquid formulation are pre-sterilized prior to spraying or dripping. In embodiments, the step of spraying or dripping is repeated to overlay one or more thin films on top of each other to fill the vial to any desired level up to totally full.

### V. Examples

The following examples as well as the figures are included to demonstrate preferred embodiments of the disclosure. It should be appreciated by those of skill in the art that the techniques disclosed in the examples or figures represent techniques discovered by the inventors to function well in the practice of the disclosure and thus can be considered to constitute preferred modes for its practice. However, those of skill in the art should, in light of the disclosure, appreciate that many changes can be made in the specific embodiments which are disclosed and still obtain a like or similar result without departing from the spirit and scope of the disclosure.

### Example 1: Dry Powder Compositions for Intranasal Delivery of Vaccines Containing a Liposomal Adjuvant

a. Materials

Chitosan (medium molecular weight), sodium alginate, gelatin, sodium carboxymethylcellulose (CMC), albumin from chicken egg white (Ovalbumin), lipid A, monophosphoryl from Salmonella enterica serotype minnesota Re 595 (MPL), and porcine mucin type III were from Sigma-Aldrich. Dulbecco's phosphate-buffered saline (DPBS) was from Gibco. QS-21 was from Dessert King International (San Diego, CA). 1,2-Dioleoyl-sn-glycero-3-phosphocholine (DOPC) was from Avanti Polar Lipids (Alabaster, AL). Cholesterol was from MP biomedicals (Irvine, CA).

b. Preparation of the AdjLMQ/OVA Model Vaccine.

The AdjLMQ/OVA model vaccine was prepared as previously described (AboulFotouh et al., 2022). Briefly, the liposome formulation was prepared by dissolving 1 mg DOPC, 0.25 mg of cholesterol, and 50 μg of MPL in 2 mL ethanol. A lipid film was formed by solvent evaporation under a gentle stream of nitrogen gas. The lipid film was then hydrated with phosphate buffered saline (PBS, 10 mM, pH 7.2) to form liposomes. QS-21 (50 μg) in aqueous solution was added to the liposomes and the volume was adjusted to 0.5 mL using PBS (10 mM, pH 7.2). AdjLMQ-adjuvanted OVA (AdjLMQ/OVA) model vaccine was prepared by mixing OVA, dissolved in PBS, with AdjLMQ at a concentration of 50 μg OVA/0.5 mL.

C. Preparation of AdjLMQ/OVA Model Vaccine with Different Mucoadhesive Agents.

To prepare the AdjLMQ/OVA model vaccine with different concentrations of chitosan, sodium alginate, gelatin, or CMC, 50 g of QS-21, 50 g of OVA, and 19.5 mg of sucrose were added to 125 μL of the liposome dispersion. The stock solution of chitosan was prepared by dissolving chitosan 2% (w/v) in 0.1 M acetic acid aqueous solution. The stock solutions of sodium alginate, alginate, and CMC were prepared by dissolving them (2% w/v) in DPBS. Different volumes of the mucoadhesive agent-containing solutions were then added to the concentrated AdjLMQ/OVA vaccine to achieve a final concentration of 0.1%, 0.2%, 0.4%, or 1% w/v, corresponding to 1.9%, 3.7%, 7.1%, or 16.0% w/w of the mucoadhesive agent vs. the theoretical total weight of all components, except water. The final volume of the AdjLMQ/OVA with different concentrations of mucoadhesive agents was then adjusted to 0.5 mL with DPBS. The vaccine preparations with different concentrations of mucoadhesive agents were converted into dry powders by TFFD as previously described (Xu et al., 2021). Briefly, the formulations were dropped into a dry ice cooled serum vial to form frozen thin films. The vial was then preserved in a −80° C. freezer until lyophilization using a lyophilizer (VirTis Advantage). A 60 h lyophilization cycle performed at pressures ≤80 mTorr could be divided into three stages: (1) The shelf temperature was maintained at −40° C. for 20 h, (2) gradually ramped to 25° C., over 20 h, and (3) maintained at 25° C. for 20 h. The vials were then back filled with nitrogen gas and stored at room temperature. The particle size of the vaccines before and after being subjected to TFFD and reconstitution (with water) was measured by dynamic light scattering (DLS) using a Malvern Nano ZS. Samples were diluted 50 times with PBS before the measurement. The pH value of all the samples was approximately 7.4.

### D. Evaluation of the Integrity of the OVA.

SDS-PAGE was applied to evaluate the integrity of OVA in AdjLMQ/OVA model vaccine compositions with 0%, 1.9%, or 3.7% of CMC before and after TFFD. Vaccine powders were reconstituted with water. The samples were mixed with Laemmli sample buffer 4× containing 10% 2-mercaptoethanol and boiled for 10 min at 100° C. Finally, 30 μL of each sample were loaded into the wells of a 4-20% precast polyacrylamide gel (Bio-Rad). The electrophoresis was performed at 90 V for 90 min. The SDS-PAGE gel was stained with Coomassie G-250, and the image was captured with a camera.

### E. In Vitro Mucoadhesion Test of the TFFD Vaccine Powders.

The in vitro mucoadhesion test was done following a previously reported procedure with modifications (Trenkel & Scherließ, 2021). To simulate the human nasal mucosal tissue, 100 mm square Petri dishes were coated with 1.5% (w/v) agar plus 2% (w/v) porcine mucin in DPBS (pH=6). The coating layer was solidified by incubating the Petri dishes for 2 h at room temperature. The dishes were then stored at 4° C. To track the movement of the TFF AdjLMQ/OVA thin films on the agar gel, 25 μL of the DPBS was replaced with 25 μL of DPBS containing crystal violet (1% w/v) when preparing the vaccine. To make each film with a similar geometry, the distance between the needle and the bottom of the vial was fixed, and the number of droplets per vial was limited to 5 when performing TFF.

The coated dishes were incubated at 32° C. in a Fisher Scientific incubator-shaker (Hampton, NH) until equilibrium (shaking not applied), and the TFF vaccine films were gently placed onto the gel (1 film per spot). The dishes were then turned vertically and incubated for 10 min at 32° C. The maximum downward movement of the vaccine film was measured using a ruler.

### F. Deposition Pattern of the TFFD Vaccine Powder

For this experiment, the OVA in the vaccine formulation was replaced with FITC-labeled OVA-FITC (i.e., AdjLMQ/OVA-FITC with 1.9% CMC). Two 3D-printed nasal casts based on the CT-scan images of a 48-years old male and a 7-years old female were used to perform the deposition test (Warnken et al., 2018). The inner surfaces of the nasal casts were coated with a simulated nasal mucus fluid (Bio Chemazone, Alberta, Canada), and the excess fluid was purged with air. Then, about 20 mg of the TFFD AdjLMQ/OVA-FITC with 1.9% CMC was loaded into a BD 1 mL syringe, which was then sprayed into the left nostril of the nasal casts by rapidly pushing the syringe plunger to reach a powder initial velocity of about 200 cm/s. The coronal angle, sagittal angle, and insertion depth of the syringe were 0°, 45°, and 0.5 cm, respectively. To determine the effect of breathing on the deposition pattern, the nasal cast was connected to a vacuum pump, and the flow rate was adjusted to 0 or 10 liters per minute (LPM). The nasal casts were then disassembled into the following parts: (1) anterior, (2) upper turbinate, (3) middle turbinate, (4) lower turbinate, (5) nasopharynx, and (6) filter. Each part was then rinsed with 5 mL of water, and the fluorescence intensities of the samples were measured with a microplate reader from BioTek (Winooski, VT).

### Results

### A. Screening of the Potential Mucoadhesive Agents.

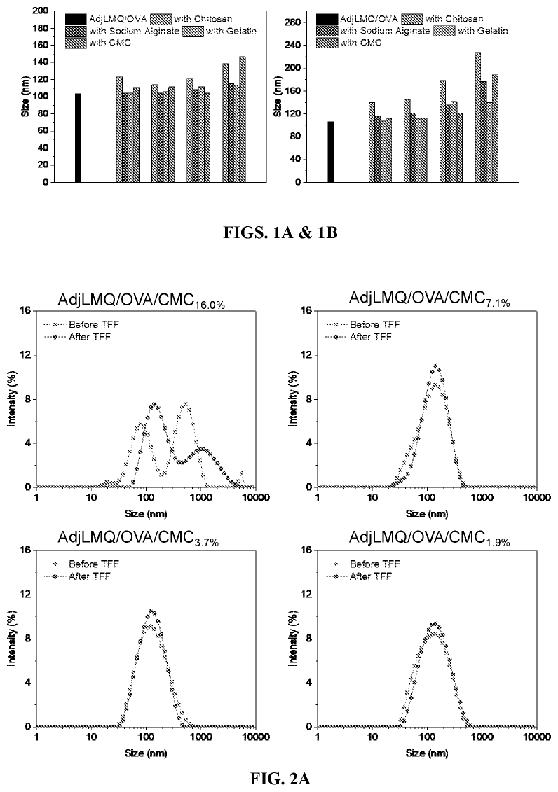

To render the vaccine mucosal adhesive, different mucoadhesive agents, chitosan, sodium alginate, gelatin, or CMC, were added to the AdjLMQ/OVA vaccine. The final concentrations of the mucoadhesive agents tested in the powders ranged from 1.9% to 16% by weight. FIG. 1A shows the particle sizes of the AdjLMQ/OVA vaccine containing different concentrations of the mucoadhesive agents before being subjected to TFFD. Sodium alginate, gelatin, and CMC did not significantly affect the particle size of AdjLMQ/OVA vaccine, while chitosan caused a significant particle size increase, even when the concentration was only 1.9% by weight. Moreover, chitosan and CMC caused a significant particle size increase at 16.0% by weight (FIG. 1A).

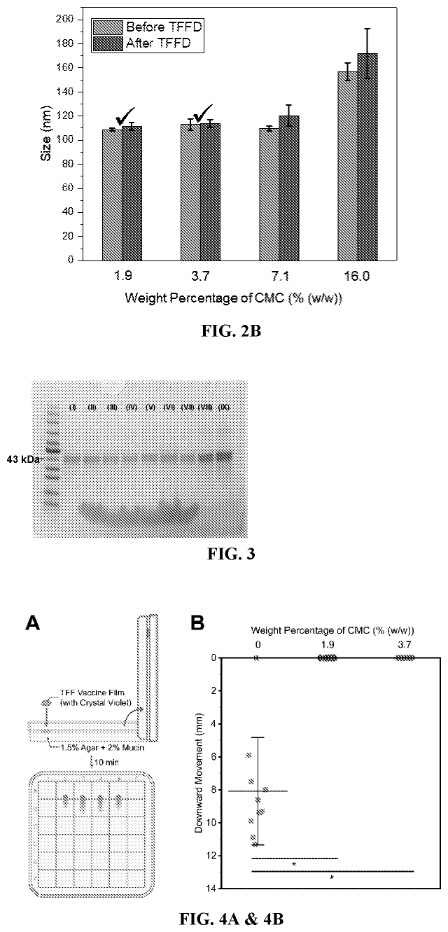

After TFFD, only the vaccine formulations contained gelatin or CMC (and too a less extent, the formulations contained sodium alginate) at 1.9% or 3.7% by weight maintained their particle size upon reconstitution (FIG. 1B). The AdjLMQ/OVA vaccine formulations with CMC at 1.9% or 3.7%, w/w were selected for further analysis. Detailed analysis of the particle size is shown in FIG. 2.

### B. The Integrity of the OVA in the AdjLMQ/OVA Vaccines Before and After Being Subjected to TFFD.

To evaluate the impact of the TFFD process on the integrity of OVA, SDS-PAGE was used. The SDS-PAGE result of the standard OVA solutions showed two bands of around 43 kDa (FIG. 3), which might be attributed to the different glycosylated forms of OVA. A comparison of the SDS-PAGE results of the AdjLMQ/OVA vaccine with different concentrations of CMC before and after TFFD showed that the chemical integrity of OVA was maintained after TFFD (FIG. 3).

### C. In Vitro Mucoadhesion Test of the AdjLMQ/OVA Powders.

Petri dishes coated with 1.5% (w/v) agar plus 2% (w/v) porcine mucin were used to simulate the surface of human mucosal tissue. In the in vitro mucoadhesion test, films of TFF AdjLMQ/OVA powders with 0, 1.9, or 3.7% w/w of CMC and about 1% w/w of crystal violet were placed on the agar plus porcine mucin layer. The dishes were turned vertically and incubated at 32° C. (FIG. 4A). The thin films swelled due to the moisture in the gel or the atmosphere and moved down slowly. The maximum traveling distances of the formulations at 10 min were then recorded. FIG. 4B shows the downward movement of all three TFF vaccine powders. Only the TFF AdjLMQ/OVA powders without CMC moved (8.1±3.3 mm) after 10 min of incubation. The TFF AdjLMQ/OVA powders with 1.9% or 3.7% w/w CMC did not move, indicating that they were adhesive to the porcine mucin-containing agar surface. Considering there is no difference between TFF AdjLMQ/OVA vaccine with 1.9% and 3.7% w/w CMC (FIG. 4B), the AdjLMQ/OVA with 1.9% CMC (AdjLMQ/OVA/CMC_{1.9%}) was selected for further studies.

D. Characterization of the AdjLMQ/OVA without or with 1.9% CMC.

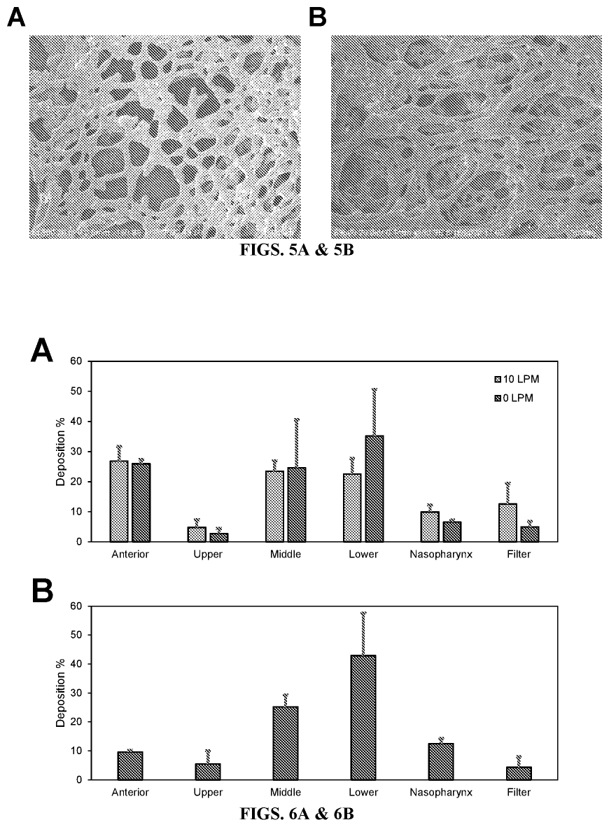

The surface morphology of the TFFD AdjLMQ/OVA and AdjLMQ/OVA with 1.9% CMC was characterized using SEM. The SEM images (FIG. 5) revealed that the TFFD AdjLMQ/OVA and AdjLMQ/OVA with 1.9% CMC powders were porous. Also, no phase separation was observed in both formulations.

### E. Deposition Pattern of the Thin-Film Freeze-Dried Vaccine Powder in Nasal Casts.

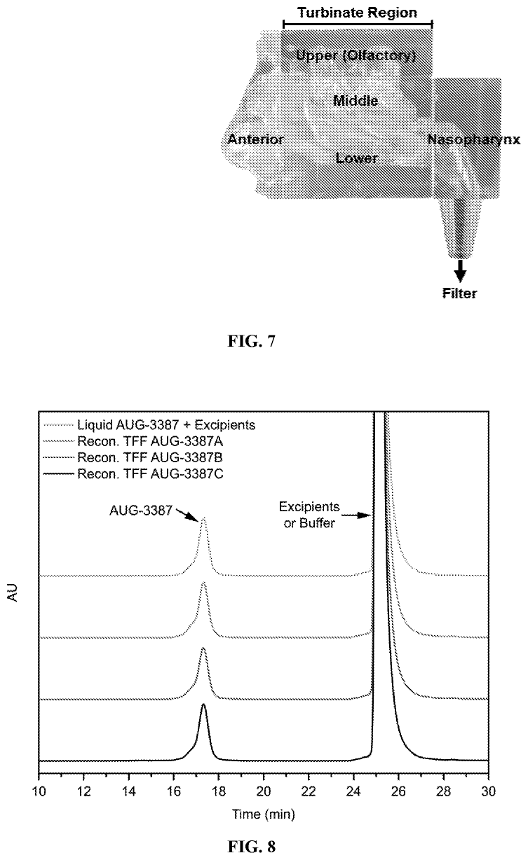

The deposition pattern of the thin-film freeze-dried AdjLMQ/OVA-FITC with 1.9% CMC powder in the nasal cast based on the CT scan of a 48-years old male is shown in FIG. 6A. When the coronal angle and the sagittal angle were 0° and 45°, respectively, the majority of the TFFD powder deposited in the anterior and turbinate regions without air flow. However, when an air flow of 10 LPM was applied to the nasal cast, the deposition percentage in the nasopharynx and filter region increased, which is expected. Nonetheless, the percentage deposited in the upper turbinate region where the olfactory is located was less than 10% in both conditions, indicating that the exposure of such vaccine powder to the olfactory can be minimal. We then repeated the experiment with a nasal cast based on the CT scan of a 7-years old female, while the flow rate was fixed at 0 LPM (FIG. 6B). The result indicated that most of the thin-film freeze-dried AdjLMQ/OVA-FITC with 1.9% CMC powder was deposited in the turbinate region and the nasopharynx region. Compared to the results in the adult nasal cast, the deposition of the powder in the anterior region was significantly lower in the children nasal cast, likely due to the shorter nasal cavity in the children nasal cast. A graphic representation of each of these regions can be seen in FIG. 7.

### Example 2—Monoclonal Antibody Dry Powder Compositions for Intranasal Delivery

Materials: AUG-3387 mAb was from TFF Pharmaceuticals, Inc. (Austin, TX). L-histidine and fluorescein isothiocyanate isomer I (FITC) were from Acros Organics (Geel, Belgium). D-mannitol, Bradford reagent, and Tween 20 were from Sigma-Aldrich (St. Louis, MO). Leucine was from Spectrum Chemical Mfg. Corp. (New Brunswick, NJ). Artificial nasal mucus was from Biochemazone (Leduc, Canada).

Preparation of AUG-3387 dry powders: AUG-3387 was dialyzed overnight in a histidine buffer (20 mM, pH 6) containing 0.02% of Tween 20. The composition of the histidine buffer was based on Haeuser et al. (2020). Then, histidine buffer containing mannitol and leucine were added to the mAb solution according to the ratios in Table 1. Histidine buffer was used to adjust the final volume. The liquid mAb formulations were then converted to powders by thin-film freeze-drying. Briefly, the liquid formulations were dropped onto the surface of a rotating cryogenically cooled stainless steel drum with a BD 1 mL syringe equipped with a 21 G needle. The temperature of the drum was controlled at −70 to −100° C. The frozen films were collected into a container containing liquid nitrogen and transferred to 5 mL glass vials. The vials were semi-stoppered, and the formulations were dried using a SP Scientific Virtis Advantage Pro lyophilizer (Warminster, PA). The lyophilization process consisted of a 20 h primary drying step at −40° C., a ramping step from −40° C. to 25° C. over 20 h, and a 20 h secondary drying step at 25° C. The chamber pressure of the lyophilyzer was maintained at 80 mTorr. After the lyophilization process, the vials were back-filled with nitrogen, stoppered, crimped, and then stored at room temperature.

TABLE 1
AUG-3387 mAb formulations with different solid contents.
The final volume was adjusted to with histidine buffer.

| | AUG-3387A | AUG-3387B | AUG-3387C |
|---|---|---|---|
| AUG-3387 (g/L) | 1.49 | 2.98 | 4.47 |
| Mannitol (g/L) | 8.03 | 16.06 | 24.09 |
| Leucine (g/L) | 0.423 | 0.846 | 1.269 |
| Final volume (μL) | 500 | 250 | 167 |

Size exclusive chromatography analysis: An Agilent 1260 Infinity II liquid chromatography (LC) system (Santa Clara, CA) equipped with an AdvanceBio SEC 300 Å2.7 μm 7.8×300 mm column was used to evaluate mAb monomer and high molecular weight (HMW) species. The mAb samples were diluted to 0.298 mg/mL, filtered through a 2.0 μm glass microfiber syringe filter and a 0.45 μm polyethersulfone (PES) syringe filter, and stored in a refrigerator. The HPLC conditions were sample volume, 5 μL; mobile phase, 0.15 M phosphate buffer (pH=7); run time, 40 min; flow rate, 0.5 mL/min; and detection wavelength, 220 nm. The peak area of the mAb on the chromatogram was determined using Agilent OpenLab Software, and the numerical data of the chromatogram was exported with a UniChrom software.

Micro-flow imaging (MFI) analysis: MFI was applied to analyze proteinaceous particles in the mAb formulations. The TFF mAb formulations were first reconstituted to the original volume with water. Histidine buffer was then used to dilute the samples to reach a final mAb concentration of 0.310 mg/mL. The samples were analyzed using an MFI 5100 micro-flow imaging system (ProteinSimple, San Jose, CA) with an auto-sampler. The analyzed sample volume was 0.35 mL. The spread sheet containing the equivalent circular diameter (ECD), area, perimeter, circularity, aspect ratio, and intensity standard deviation profiles of each detected particles were exported with the ProteinSimple MVSS software. To exclude the contaminant particles such as silicone oil droplets and air bubbles, a filter was applied to exclude the particles with the aspect ratio ≥0.8 and intensity standard deviation >100 according to the literature (Guo et al., 2022). Also, the particles presence on the edge of the images were removed, while the “remove slow and stuck particles” feature was not applied.

The particle counts were converted to mass of proteinaceous particles following the ellipsoid-volume (E-V) method by Kalonia et al. (2015). Briefly, the mass of an individual particle k (mₖ) was first determined using following equation (1):

$${m}_{k} = \frac{π}{6} \times {d}^{3} \times \sqrt{1.70763 - 1.70864 \times {( 1 - C )}^{0.45} - 0.70638 C} \times 1.41 g / mL \times f \qquad ( 1 )$$

Where d is the ECD of the particle, C is the circularity of the particle, and f is the protein fraction of the particle. A value of 0.2 was chosen for f by assuming proteinaceous particles contain 20% protein and 80% solvent.
The total mass of proteinaceous particles (M) were then calculated following equation (2):

$$M = {\sum }_{k = 1}^{n} {m}_{k} \qquad ( 2 )$$

Where n is the number of particles.

Finally, the mass percentage of the particles was calculated using following equation (3), in which we divided the M with the theoretical concentration of mAb in the solution (i.e., 0.3104 mg/mL) multiplied by the volume being analyzed (Vₐ).

$$Mass percentage of the particles ( \% ) = \frac{M ( mg )}{0.3104 ( mg / mL ) \times {V}_{a} ( mL )} \times 100 \% \qquad ( 3 )$$

Measurement of the water content: The water content in the TFF AUG-3387C powder was determined by Karl Fischer titration using a Mettler Toledo C20 coulometer (Columbus, OH). Briefly, 1 mL of the HYDRANAL™-Coulomat AG solution (Honeywell, Charlotte, NC) was drawn from the buffer tank and injected into the vial containing the TFF mAb powder. The TFF AUG-3387 powder was dissolved with the Coulomat AG solution, and 0.5 mL of the mixture was injected back into the buffer tank. The water content (%) of the sample was then calculated using the following equation (4):

$$Water moisture ( \% ) = \frac{Measured water content ( mg )}{Weight of the sample ( mg )} \times 100 \qquad ( 4 )$$

Measurement of the specific surface area: The specific surface area of the TFF mAb powder was measured using a Brunauer-Emmett-Teller (BET) surface area analysis. The sample was loaded into the sample cells and outgassed under helium flow for 1 day. The nitrogen absorption was then performed with p/p0 ranging from 0.1 to 0.3 with an Anton Paar Quantachrome AutoFlow BET+ instrument (Graz, Austria). The specific surface area of the TFF mAb powder was then calculated with the BET method.

X-ray diffraction (XRD): The TFF AUG-3387 and TFF mAb powders were mounted with mineral oil and loaded onto a sample loop. The 2D XRD pattern of the sample was measured with a Rigaku Spider instrument (Tokyo, Japan). The background of the sample's 2D diffraction pattern was subtracted using the diffraction pattern of mineral oil. The 2D diffraction pattern was then transformed into 1D pattern and the 1D pattern was smoothed with the Rigaku 2D Data Processing (2DP) software. The crystallographic information files (CIF) of the mannitol (Fronczek et al., 2003), leucine (Coll et al., 1986), and histidine (Madden et al., 1972a; Madden et al., 1972b) were obtained from the Cambridge Crystallographic Data Centre (CCDC), and the simulated XRD patterns were generated with the CCDC mercury software.

Scanning electron microscopy: The scanning electron microscopic (SEM) images of the TFF AUG-3387C powder was taken with a Hitachi S-5500 field emission SEM instrument (Tokyo, Japan). The bulk TFF powder was fixed on the sample stub with a carbon tape. The sample was coated in a sputter coated equipped with an Au/Pd (60:40) target from Electron Microscopy Sciences (Hatfield, PA). The SEM images were then acquired with an acceleration voltage of 30 kV.

Deposition pattern of the TFF AUG3387 powder: FITC-labeled AUG-3387 mAb was used to prepare the TFF AUG-3387C powder for the deposition studies. The AUG-3387 mAb was labeled with FITC following the instruction of the Sigma-Aldrich FluoroTag FITC conjugation kit, and the product was purified by ultrafiltration. The TFF AUG-3387C powder was loaded into a BD 1 mL TB syringe equipped with 21 G×1 (0.8 mm×25 mm) needle and sprayed into the nasal replica casts 3D-printed based on the CT-scan images of the nasal cavities of a 48-year-old adult male or a 7-year-old child female. Before spraying the powder, the nasal replica cast was coated with simulated nasal mucus. A Cytiva 2 μm glass microfiber syringe filter (Marlborough, MA) was connected after the nasopharynx part. The powder was sprayed into the left nostril of the replica casts. The coronal angel was fixed at 0°, and the sagittal angle was fixed at 45°. The insertion depth for the adult and child nasal casts was 0.5 cm. To study the effect of the flow rate on the deposition, for the adult nasal cast, the flow rate of 0 and 10 L/min were studied, and for the child nasal cast, flow rates of 0 and 3 L/min were studied.

After the powder was sprayed, the replica cast was dissembled into anterior, upper, middle, and lower turbinate, nasopharynx, and filter parts. Each part was rinsed with 5 mL of water, and the fluorescence intensity of the FITC-labeled TFF AUG-3387C (λ_{EX}=485 nm, λ_{EM}=528 nm) was quantified using a BioTek Synergy Microplate Reader (Winooski, VT). A calibration curve was used to convert the fluorescence intensity into mAb concentration.

Statistical analysis: All the data are presented as mean±standard deviation (SD). One-way analysis of variance (ANOVA) followed by Tukey's post hoc was performed using Microsoft Office Excel with the Real Statistics Resource Pack (Release 8.5).

Results. Preparation and characterization of TFF mAb powders: AUG-3387 is a human-derived mAb against SARS-CoV2. Previously AUG-3387 mAb has been thin-film freeze-dried with mannitol and leucine (95:5) as the excipients into dry powders with desired aerosol properties for pulmonary delivery into the lungs, while preserving its integrity and activity (Emig et al., 2021). Here, the composition has been modified by changing the buffer from the phosphate-buffered saline (PBS) to a histidine buffer and including a small amount of Tween 20 (Table 1). Three liquid formulations, AUG-3387A, AUG-3387B, and AUG-3387C, were prepared with final mAb concentrations at 1.49 g/L, 2.98 g/L, or 4.47 g/L, respectively, while the concentration of the buffer and Tween 20 remained the same. The mAb liquid formulations were then subjected to thin-film freeze-drying to produce three different dry powders.

Characterization of the TFF mAb powders: The water contents in the TFF mAb powders ranged from 1.68% to 2.32%. Aggregation of protein-based therapeutic agents such as mAbs after they are subjected to freezing and/or drying stresses is a key concern. Therefore, the formation of high molecular weight (HMW) species were evaluated after the mAbs were subjected to thin-film freeze-drying using size exclusion chromatography (SEC). HMW species were not detectable in the AUG-3387 liquid formulation (FIG. 8) and subjecting the mAbs to thin-film freeze-drying did not appear to cause an increase in HMW species in any of the AUG-3387 formulations (FIG. 8), indicating that the TFF process did not cause the formation of significant HMW species in any of the AUG-3387 formulations.

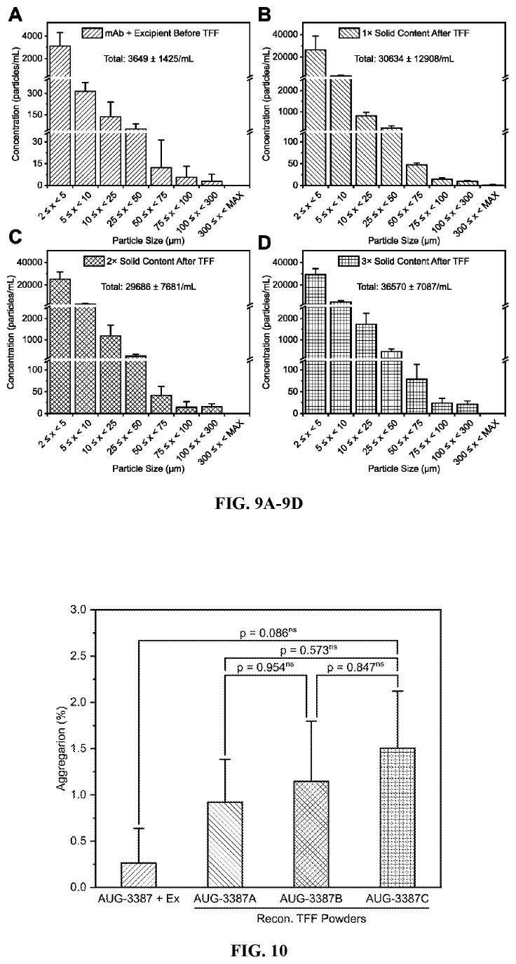

Recently, MFI has emerged as an attractive method to quantify the subvisible particles (SVPs) or proteinaceous particles in protein formulations (Sharma et al., 2010a). By combining a digital microscope with a fluidic system, the images of each particle in a protein formulation can be captured, and the information of each particle such as ECD, circularity, and aspect ratio can be calculated with an image analysis software (Sharma et al., 2010b). Shown in FIGS. 9A-D are the counts of particles in the ranges of [2 mm, 5 mm), [5 mm, 10 mm), [10 mm, 25 mm), [25 mm, 100 mm), and 100 mm or larger in the TFF mAb powders upon reconstitution. Particles of larger than 100 mm are considered visible, and those that are smaller than 100 mm are considered subvisible. In products such as injectable protein products, there are recommended limits for the number of particles larger than 10 mm and larger than 25 mm (Glücklich et al., 2020), though such limits are unknown for products intended for intranasal or pulmonary administration. For all the powders, most of the SVPs were in the ranges of 2≤x<5 μm and 5≤x<10 μm. Subjecting the mAb formulations to the TFF process caused an increase in the concentration of SVPs, but the concentrations of SVPs in all three TFF AUG-3387 powders were not different (FIG. 10). At least two methods have previously been applied to calculate the mass of the proteinaceous particles using particle count data from MFI. In the method by Barnard et al. (2011), it is assumed that the proteinaceous particles are spherical, the volume fraction of the proteins in the particles is 75%, and the density of the protein is 1.43 g/mL. However, this method often overestimates the mass of protein aggregates, and the sum of the calculated mass is usually higher than the experimentally determined value (Kalonia et al., 2015). In the E-V method reported by Kalonia et al. (2015), the 2D images of the proteinaceous particles are converted into the prolate ellipsoids of revolution, and the masses of the particles are then calculated by assuming the volume fraction of the proteins in the particles is 20% and the density of the protein is 1.41 g/mL. The mass of proteinaceous particles in each of the TFF mAb powders was estimated following Kalonia et al. (2015). As shown in FIG. 10, the mass percentage of the particles in all three TFF AUG-3387 mAb powders was lower than 2%, and there was not a significant difference among those three TFF mAb powders in particle mass.

According to the Karl Fischer titration, the water contents of the TFF AUG-3387A, AUG-3387B, AUG-3387C powders were 1.6±0.3%, 2.0±0.5%, and 2.3±0.7%, respectively. Since all three powders were similar in their moisture contents and the concentrations of HMW species and the SVP masses were not different among them upon reconstitution, AUG-3387C was used for additional studies. The TFF AUG-3387C powder also had the highest mAb content, allowing the filling of more mAbs in a device for intranasal spraying.

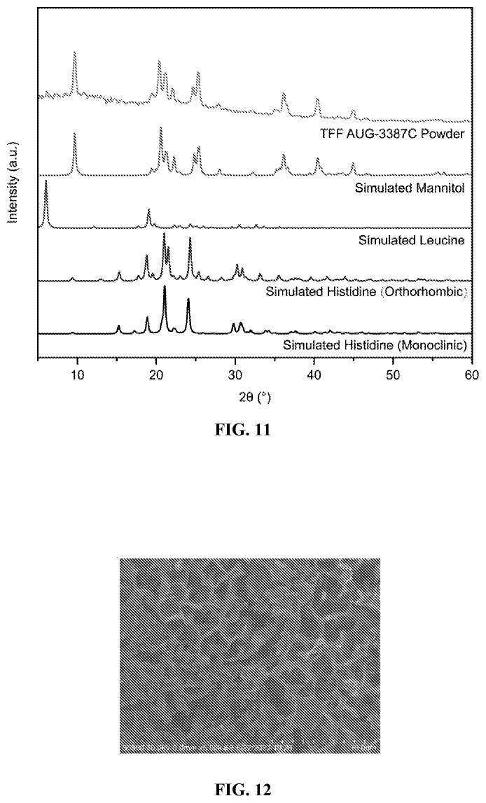

The average BET specific surface area of the TFF AUG-3387C powder was 24.934±3.354 m²/g, indicating that it was porous. FIG. 11 shows the X-ray diffractogram of the TFF AUG-3387C powder, in which most of the diffraction peaks match the simulated peaks of the crystalline mannitol (S form). The peaks of crystalline leucine are not easily identifiable, although freeze-dried leucine is usually crystalline, likely because leucine accounted for only 5% of the excipients. The histidine from the histidine buffer was also amorphous in the TFF AUG-3387C powder. The diffraction patterns of two possible form of histidine crystals were simulated to confirm the absence of crystalline histidine (FIG. 11). It is generally believed that crystals could damage mAbs during freezing. It is unclear why the mannitol crystals did not cause more aggregation of the AUG-3387 mAbs than observed, although the “particle isolation hypothesis” posits that mannitol may offer partial protection against particle aggregation by separating particles from one another within the unfrozen fraction during freezing (Allison et al., 2000). Of course, histidine in the formulation is a known cryoprotectant (Mohammed et al., 2007).

The morphology of the TFF AUG-3387C powder was examined using scanning electron microscopy. The TFFD mAb powder was porous (FIG. 12), which explains its relatively high specific surface area.

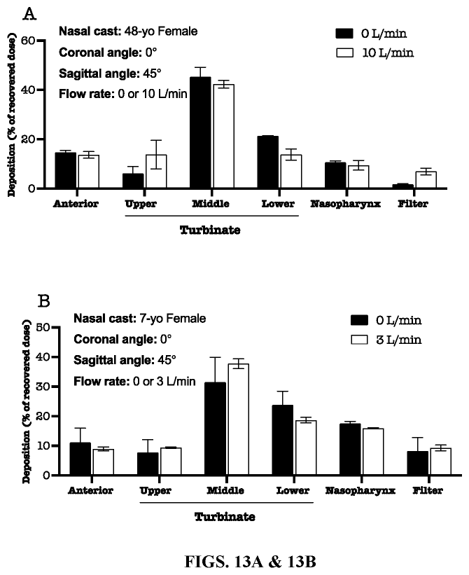

Deposition patterns of TFF AUG-3387C powder in nasal replica casts: The deposition patterns of the TFF AUG-3387C powder in nasal replica casts based on the CT-scans of an adult and a child were studied using FITC-labeled AUG-3387. FIG. 13A shows the deposition pattern of the TFF AUG-3387C powder in the nasal replica cast of a 48-year-old male. Most of the powder was deposited in the middle and lower turbinates and the nasopharynx region. FIG. 13B shows the deposition pattern of the TFF AUG-3387C powder in the nasal replica cast of a 7-year-old female. Again, most of the powder was deposited in the middle and lower turbinates and the nasopharynx region, although the deposition in the nasopharynx region was the highest. Moreover, applying a flow rate (i.e., 10 L/min in adult cast and 3 L/min in child cast) did not significantly affect the deposition patterns, except in the filter in adult cast (FIG. 13). Intranasally delivered mAbs can potentially treat or prevent several diseases located in the brain or the respiratory tract, and the target region of the mAbs in the nasal cavity depends on the sites of the diseases and the transport mechanism of the mAbs. In the case of treating disease located in the brain, pre-clinical studies in animals showed that intranasal delivery of the antibodies or antibodies-containing drugs targeting the brain has the potential to treat glioblastoma or Alzheimer's disease. Chu et al. functionalized temozolomide-loaded PLGA nanoparticles with anti-EPHA3 antibodies for nose-to-brain delivery (Chu et al., 2018). They demonstrated that intranasal administration could more efficiently deliver the drug into the brain of glioma-bearing rats than intravenous administration. Musumeci et al. utilized chitosan-coated poly(lactic-glycolic acid) (PLGA) nanoparticles or nanostructured lipid carriers (NLC) to carry mAb-neutralizing tumor necrosis factor-related apoptosis-inducing ligand (TRAIL) for the treatment of Alzheimer disease (Musumeci et al., 2022). Their results indicated that through intranasal administration, the drug complexes could efficiently reach the brain of the mice. Although these studies did not specify the targeted region in the nasal cavity of the mAb, literature mentioning the general transportation mechanism of the pharmaceutics from the nasal cavity to the brain is available. Patel et al. summarized that drugs can be transported via the olfactory, trigeminal, and systemic pathways (Patel et al., 2022). Specifically, olfactory epithelium should be targeted in the olfactory pathway, maxillary and ophthalmic nerves should be targeted in the trigeminal pathway, and the capillary blood vessels in the nasal mucosa should be targeted for the systemic pathway (Patel et al., 2022).

In the case of respiratory tract diseases, intranasal delivery of the mAb can passively immunize the respiratory tract mucosal surface and prevent viral infections (Mazanec et al., 1992; Weltzin and Monath, 1999). Ye et al. (2010) delivered mAb specific to H5 hemagglutinin intranasally to the mice before or after they were inoculated with a sublethal dose of H5N1 influenza viruses. Their results indicate that intranasal delivery of mAb could provide protection against H5N1 virus infections (Ye et al., 2010). Weltzin et al. delivered a mAb that neutralizes respiratory syncytial virus (RSV) intranasally to the mice. Their results indicate that intranasal delivery of mAb prior to RSV challenge can protect the mice from both nasal and lung infections. Interestingly, the protection against lung infection can last as long as 3 days, and the mAb was still present in the lungs for 4 days, while the protection against nasal before RSV challenge cannot last for one day, which is likely because of the rapid clearance of the nasal mucus into the nasopharynx (Weltzin et al., 1994). In another study, Weltzin et al. delivered the same mAb intranasally to the Rhesus monkeys daily 2 days before and 4 days after RSV challenge and showed that the mAb protected the monkeys from infections (Weltzin et al., 1996). They also found that the mAb remained in nasal secretions for a few days after intranasal administration, while neutralizing concentration remained for more than one day after the treatment (Weltzin et al., 1996).

Recently, the outbreak of COVID-19 raised interest in passive immunization by applying intranasal mAbs. Halwe et al. (2021) indicated that intranasal delivery of DZIF-10c, a neutralizing antibody against SARS-COV-2, can protect hACE2-transduced mice from infection (Halwe et al., 2021). However, they did not analyze the exact biodistribution of the DZIF-10c following intranasal administration. Ku et al. (2021) delivered an engineered immunoglobulin M (IgM) neutralizing antibody (IgM-14) to the nasal cavities of mice for the prophylactic treatment of COVID-19. To study the biodistribution of IgM-14 following intranasal administration, they labeled the IgM-14 with Alexa Fluor 750 (AF750) dye and applied an IVIS imager to determine its distribution in vivo and ex vivo (Ku et al., 2021). Their results show that following intranasal administration, IgM-14 has long-term retention in both the nasal cavity (168 h) and lung (96 h), which conferred protection against respiratory infection since nasal epithelium is the primary location where SARS-CoV-2 starts to infect, and the virus can be aspirated into the lungs (Hou et al., 2020; Ku et al., 2021).

### Example 3—Dry Powder Compositions of CpG Oligodeoxynucleotides-Adjuvanted Vaccines for Intranasal Delivery

Materials: Class B CpG 1826 ODNs specific for murine Toll-like receptor (TLR) 9 were from InvivoGen (San Diego, CA). Ovalbumin and Bradford reagent were from Sigma-Aldrich (St. Louis, MO). Fluorescein isothiocyanate isomer I (FITC) was from Acros Organic (Geel, Belgium). Anhydrous lactose and leucine were from Spectrum Chemical Mfg. Corp. (Gardena, CA). Simulated nasal mucus was from Biochemazone (Leduc, Canada).

Preparation of CpG 1826/OVA dry powder: The liquid formulation of the model vaccine comprised CpG 1826 ODNs (16 nmol/mL), lactose (0.48% w/v) and leucine (0.32% w v) in Tris-EDTA buffer (10 mM Tris, 1 mM EDTA, pH 8.0). FITC-OVA was added to a final concentration of 100 μg/mL as a model antigen. The liquid formulation was then converted to dry powder using thin-film freeze-drying. The temperature of the drum was controlled at −80° C. The vials were semi-stoppered, and the formulations were dried in an SP Scientific Virtis Advantage Pro lyophilizer using the lyophilization cycle described in Example 1.

Deposition patterns of the CpG 1826/OVA dry powder in nasal casts: CpG 1826/OVA dry powder (OVA labeled with FITC) was loaded into a BD 1 mL TB syringe equipped with 21 G×1 (0.8 mm×25 mm) and sprayed into the nasal replica casts 3D-printed based on the CT-scan images of the nasal cavities of a 48-year-old male or a 7-year-old female as described in Example 1. The coronal angel was fixed at 0°, and the sagittal angle was fixed at 45°. The insertion depth for the adult and child nasal casts was 0.5 cm. For the adult nasal cast, a flow rate of 0 or 10 L/min was applied, and for the child nasal cast, a flow rate of 0 or 3 L/min was studied. After the powder was sprayed, the replica cast was dissembled into anterior, upper, middle, and lower turbinate, nasopharynx, and filter parts. Each part was rinsed with 5 mL of milli-Q water, and the fluorescence intensity of the FITC-OVA (λ_{EX}=485 nm, λ_{EM}=528 nm) was quantified using a BioTek Synergy Microplate Reader (Winooski, VT). A calibration curve was used to calculate the amount of FITC-OVA powder deposited on each part.

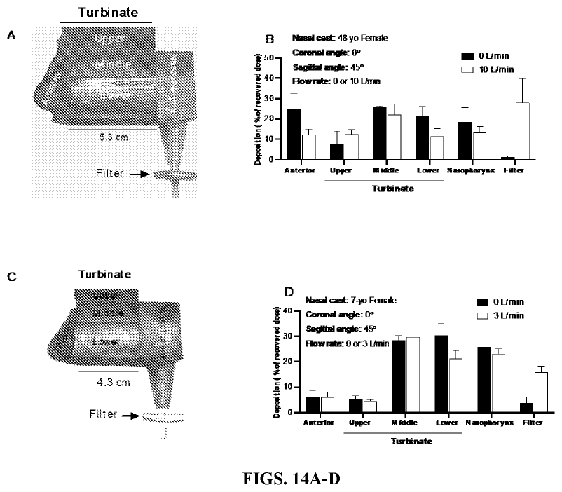

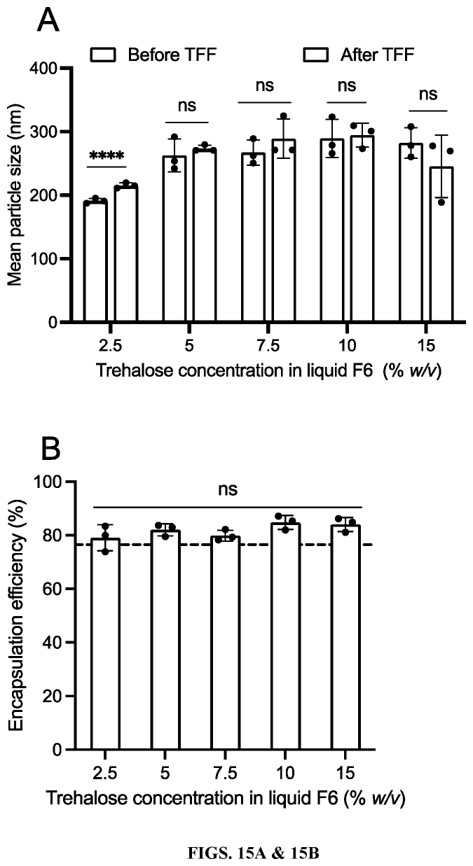

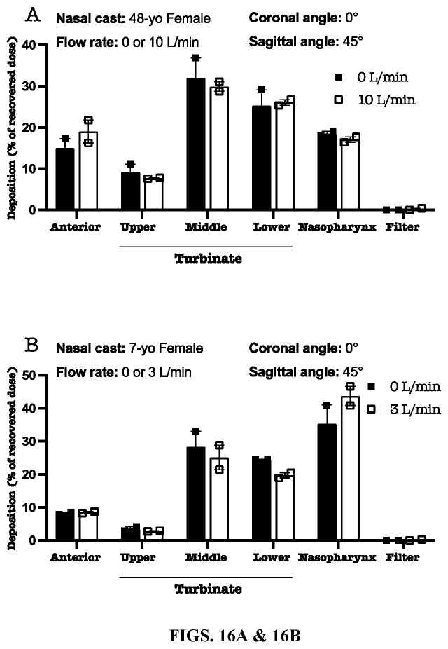

Results. At 0 L/min flow rate, about 66 or 85% w/w of the CpG 1826/OVA dry powder was deposited into the targeted regions (i.e., middle, and lower turbinate, and the nasopharynx) of the adult or child nasal cast, respectively (FIG. 14). Additionally, only ~1 or ~4% w/w of the CpG 1826/OVA dry powder was recovered from the filter connected to the nasopharynx region of the adult or child nasal cast, respectively, indicating minimal reach of the powders to the lower airways following intranasal deposition. Applying a flow rate (i.e., 10 L/min for the adult and 3 L/min for the child nasal cast) did not improve the deposition of the model vaccine in the desired regions of the nasal cavity (i.e., middle and lower turbinate regions and nasopharynx); instead it increased the recovery of the powder in the filter (i.e., about 28% and 16% w/w in the adult nasal cast and the child nasal cast, respectively), likely due to the porous, brittle matrix of the dry powder. Therefore, the CpG 1826 ODNs-adjuvanted vaccine is to be applied to human subjects intranasally, then the subjects should be advised against actively inhaling the vaccine while it is intranasally administered. Of course, the optimal application conditions need to be identified based on the nasal dry powder spraying device used.

### Example 4. Dry Powder Compositions of mRNA-Lipid Nanoparticles for Intranasal Delivery

In this example, dry powders of mRNA-LNPs for intranasal delivery were engineered using thin film freeze-drying technology. Poly(A) (MW 700-3,500 kDa) was employed as a model mRNA and trehalose was utilized as a lyoprotectant. The effect of drying on mRNA encapsulation efficiency and average particle size was explored. Additionally, the feasibility of delivering the dry powder to the desirable regions in the nasal cavity was validated by spraying the powder to nasal replica casts 3D-printed based on the CT-scan images of the nasal cavities of a 48-year-old male or a 7-year-old female.

Materials: Distearoylphosphatidylcholine (DSPC) and PEG₂₀₀₀-DSPE (MW ~2790) were from Avanti Polar Lipids (Alabaster, AL). DLin-MC3-DMA was from MedChem Express (Monmouth Junction, NJ). Cholesterol was from Sigma Aldrich (St. Louis, MO). Poly(A) was from Roche Giadnostics GmbH (Mannheim, Germany). D-(+)-Trehalose dihydrate (trehalose) was from TCI Co., LTD (Tokyo, Japan).

Formulation of poly(A)-LNPs: Poly(A)-LNPs were prepared using the nanoprecipitation technique (Whitehead et al., 2014; Hassett et al., 2021). DLin-MC3-DMA, DSPC, cholesterol and PEG₂₀₀₀-DSPE were dissolved in ethanol at a molar ratio of 40:5:38.5:0.5. Poly(A) was dissolved in RNase-free water (Thermo Fisher Scientific) at a concentration of 10 mg/mL and diluted in RNase-free citrate buffer (pH 3, 25 mM) such that the final nitrogen: phosphate (N:P) ratio of DLin-MC3-DMA:poly(A) was 3:1. The ethanolic solution of lipids and the aqueous solution of poly(A) were rapidly mixed at a ratio of 1:3 v/v, respectively, by pipetting. Following mixing, the poly(A)-LNPs were dialyzed overnight against 250× sample volume of RNase-free water using a Spectra Pro Float-A-Lyzer G2 Dialysis Device with a molecular wight cutoff of 100 kDa (Spectrum Laboratories, Inc.). The dialysis medium was exchanged twice (Davies et al., 2021). Then, the poly(A)-LNPs were diluted with trehalose in RNase-free water and RNase-free tris buffer (pH 8, final molarity 0.2 mM).

Thin film freeze-drying of poly(A)-LNPs: Poly(A)-LNPs were diluted with a trehalose in RNase-free water directly after dialysis. The final trehalose concentrations in the liquid LNP formulation were 2.5, 5, 7.5, 10 or 15%, w v. All formulations contained RNase-free Tris buffer (pH 8, 0.2 mM). The liquid poly(A)-LNP formulations were then converted to dry powder using TFFD in RNase-free environment. The temperature of the drum was controlled at −80±5° C. The vials were semi-stoppered, and the formulations were dried in an SP Scientific Virtis Advantage Pro lyophilizer. The lyophilization process consisted of a 20 h primary drying step at −40° C. and a ramping step from −40° C. to +4° C. over 1 h. The chamber pressure of the lyophilizer was maintained at 80 mTorr. After the lyophilization process, the vials were back-filled with nitrogen, stoppered, crimped, and then stored at 4° C.

Dynamic light scattering: The average particle size of poly(A)-LNPs before and after thin-film freeze-drying and reconstitution in RNase-free water was measured by dynamic light scattering (DLS) using a Malvern Zeta Sizer Nano ZS (Worcestershire, UK). Samples were diluted in PBS (pH 7.4, 10 mM) at a volumetric ratio of 1:9 prior to measurement.

mRNA encapsulation efficiency: The mRNA encapsulation efficiency was determined using the Quant-iT Ribogreen RNA assay (Invitrogen) as previously described (Hassett et al., 2021). Briefly, mRNA-LNP formulations were diluted in 10 mM Tris-HCl and 1 mM EDTA buffer (pH 7.5) in the presence and absence of 1% Triton X-100 (Sigma-Aldrich). Samples were then incubated with the Ribogreen reagent for 3-5 min at room temperature in dark. Fluorescence intensities (λ_{EX} 480 and λ_{EM} 520 nm) were measured for total mRNA bound to Ribogreen dye after release from the LNPs by Triton X-100 and unencapsulated mRNA bound to Ribogreen dye in the absence of Triton X-100.

Deposition patterns of mRNA-LNP dry powders in nasal replica casts: The thin-film freeze-dried powder of mRNA-LNP formulation that contained 2.5% w/v trehalose was loaded into a BD 1 mL TB syringe with 21 G×1 (0.8 mm×25 mm) and sprayed into the nasal replica casts 3D-printed based on the CT-scan images of the nasal cavities of a 48-year-old male or a 7-year-old female as described previously. The coronal angel was fixed at 0°, and the sagittal angle was fixed at 45°. The insertion depth for the adult and child nasal casts was 0.5 cm. For the adult nasal cast, a flow rate of 0 or 10 L/min was applied, and for the child nasal cast, a flow rate of 0 or 3 L/min was studied. After the powder was sprayed, the replica cast was dissembled into anterior, upper, middle, and lower turbinate, nasopharynx, and filter parts. Each part was rinsed with 5 mL of milli-Q water, and trehalose was quantified using an HPLC system (1220 Infinity II HPLC, Agilent, Santa Clara, CA) as previously described (AboulFotouh et al., 2022). The sugar was detected using an Evaporative Light Scattering Detector (1290 Infinity II ELSD, Agilent, Santa Clara, CA).

Statistical analysis: Student's t-test or one-way analysis of variance (ANOVA) followed by Tukey's or Dunnet multiple comparison test were performed using GraphPad Prism version 8.0.0 (GraphPad Software, San Diego, CA). Differences were deemed significant if p≤0.05.

Results. Poly(A)-LNPs were dried using thin-film freeze-drying technology. Trehalose was added to the liquid LNPs at different final concentrations (2.5, 5, 7.5, 10 or 15% w/v) as a lyoprotectant. As shown in FIG. 15A, the mean particle size of poly(A)-LNPs was maintained after thin-film freeze-drying when trehalose was employed as a lyoprotectant at a concentration in the range of 5 and 15% w/v. Similarly, trehalose at 2.5-15% w/v successfully maintained the poly(A) encapsulation efficiency after thin-film freeze-drying (FIG. 15B). It was reported that mRNA-LNPs with an average size between 200 and 500 nm can induce dendritic cell maturation and gene expression (Sasaki et al., 2022; Okuda et al., 2022). The poly(A)-LNP powder dried with 2.5% w/v trehalose was selected for intranasal deposition studies in nasal replica casts of a 48-year-old male and a 7-year-old female.

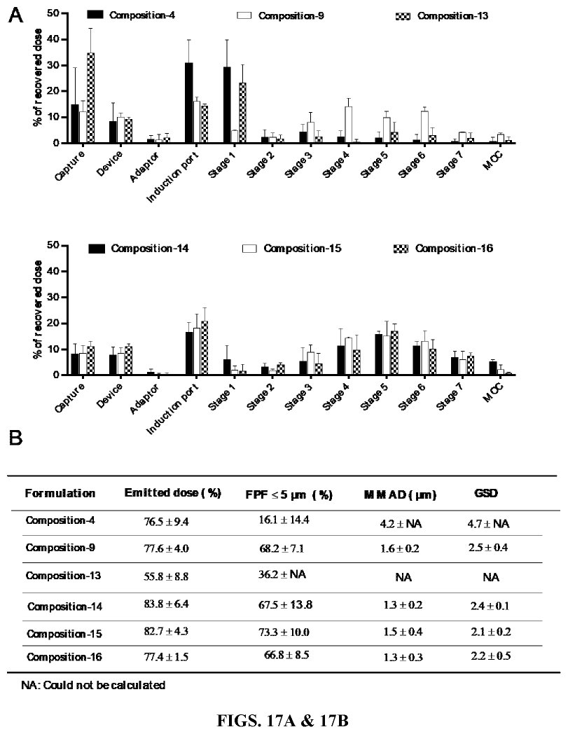

About 74% and 85% w/w of the poly(A) dry powder was deposited in the targeted regions (i.e., middle and lower turbinates and the nasopharynx) of the adult and child nasal casts, respectively (FIGS. 16A & B), regardless of the air flow applied. Additionally, almost no powder was recovered from the filter connected to the nasopharynx region of the adult or child nasal casts, indicating minimal to absence powder delivery beyond the nasal cavity following intranasal administration.

Overall, it is concluded that the mRNA-LNP dry powder prepared by thin-film freeze-drying can be delivered to the targeted regions within the human nasal cavity independent of the patients' breath pattern. If the LNPs are loaded with an antigen-encoding mRNA, converted to a dry powder by thin-film freeze-drying, and then sprayed into the nasal cavity of human subjects, then one may expect the mRNA-LNPs in dry powder to elicit desirable immune responses within the respiratory tract, in addition to specific systemic immune response.

### Example 5: Inhalable Dry Powder Compositions of CpG 1826 Oligodeoxynucleotides

Methods. Thin-film freeze-drying. Dry powders of class B CpG 1826 ODNs (InvivoGen) were prepared using thin-film freeze-drying technology [3]. Before thin-film freeze-drying, leucine and lyoprotectants were dissolved in a Tris-EDTA buffer (TE, pH 8.0, 10 mM Tris, 1 mM EDTA). Subsequently, leucine as well as a lyoprotectant selected from sucrose, trehalose, lactose, and mannitol were mixed with the CpG 1826 ODNs in sterile water based on Table 2. Liquid formulations were frozen into thin films by dropping it onto the surface of a rotating, cryogenically cooled drum using BD 1 mL TB syringe. The drum temperature was controlled at −80±5° C. The frozen thin films were collected in 5 mL-glass vials filled with liquid nitrogen and stored at −80° C. until lyophilization. Finally, semi-stoppered vials were dried using an SP Virtis Advantage Pro lyophilizer. Primary and secondary drying were accomplished at shelf temperatures of −40° C. or 25° C., respectively, for 20 h each. Shelf temperature ramping from −40° C. to 25° C. was completed over 20 h. The chamber pressure of the lyophilizer was maintained at 80 mTorr throughout the lyophilization process. After the lyophilization process, the vials were back-filled with nitrogen, stoppered, crimped, and then stored until analysis.

TABLE 2
Compositions of liquid CpG 1826 ODNs formulations.

| | CpG 1826 | Sucrose | Trehalose | Mannitol | Lactose | Leucine | TE buffer | Total solid |
|---|---|---|---|---|---|---|---|---|
| Formulation # | ODNs (% w/v) | (% w/v) | (% w/v) | (% w/v) | (% w/v) | (% w/v) | salts (% w/v) | content (% w/v) |
| Formulation-1 | 0.0026 | 0.24 | 0 | 0 | 0 | 0.16 | 0.19 | 0.59 |
| Formulation-2 | 0.00256 | 0.34 | 0 | 0 | 0 | 0.06 | 0.19 | 0.59 |
| Formulation-3 | 0.00256 | 0 | 0.24 | 0 | 0 | 0.16 | 0.19 | 0.59 |
| Formulation-4 | 0.00256 | 0 | 0.34 | 0 | 0 | 0.06 | 0.19 | 0.59 |
| Formulation-5 | 0.00256 | 0 | 0 | 0.24 | 0 | 0.16 | 0.19 | 0.59 |
| Formulation-6 | 0.00256 | 0 | 0 | 0.34 | 0 | 0.06 | 0.19 | 0.59 |
| Formulation-7 | 0.00256 | 0.48 | 0 | 0 | 0 | 0.32 | 0.19 | 0.99 |
| Formulation-8 | 0.00256 | 0.68 | 0 | 0 | 0 | 0.12 | 0.19 | 0.99 |
| Formulation-9 | 0.00256 | 0 | 0.48 | 0 | 0 | 0.32 | 0.19 | 0.99 |
| Formulation-10 | 0.00256 | 0 | 0.68 | 0 | 0 | 0.12 | 0.19 | 0.99 |
| Formulation-11 | 0.00256 | 0 | 0 | 0.48 | 0 | 0.32 | 0.19 | 0.99 |
| Formulation-12 | 0.00256 | 0 | 0 | 0.68 | 0 | 0.12 | 0.19 | 0.99 |
| Formulation-13 | 0.00256 | 0 | 0 | 0 | 0.34 | 0.06 | 0.19 | 0.59 |
| Formulation-14 | 0.00256 | 0 | 0 | 0 | 0.48 | 0.32 | 0.19 | 0.99 |
| Formulation-15 | 0.01024 | 0 | 0 | 0 | 0.48 | 0.32 | 0.18 | 0.99 |
| Formulation-16 | 0.01024 | 0 | 0.48 | 0 | 0 | 0.32 | 0.18 | 0.99 |

In vitro aerosol performance study. The aerodynamic properties of CpG 1826 ODN dry powders (Table 3) prepared using thin-film freeze-drying were evaluated using a Next Generation Impactor (NGI, MSP Corp., Shoreview, MN) as described before [4]. NGI collection cups were coated with a thin layer of 1.5% w/v Tween 20 in methanol and dried before analysis. Approximately 3-4 mg of each dry powder formulation was loaded in a size 3 hydroxypropyl methylcellulose capsule (VCaps® plus, Lonza, Inc., Morristown, NJ), which was then placed in a high resistance Plastiape® RS00 inhaler (Plastiape S.p.A, Osnago, Italy). Powders were dispersed for 4 s at a flow rate of 60 L/min providing 4 kPa pressure drop across the device. After actuation, the remaining powder in the capsule as well as powders deposited in the inhaler device, adapter, induction port, stages 1-7, and micro-orifice collector (MOC) were collected in ultrapure water. Leucine content was assayed using 2,4,6-trinitrobenzene sulfonic acid (TNBSA) to estimate the amount of powder deposited on each stage. Briefly, samples were diluted 1:1 v/v with sodium bicarbonate buffer (pH 9.0, 0.1 M). Then, freshly prepared TNBSA solution (0.01% w/v in methanol) was added to the samples in a 96-well plate. The plate was sealed and incubated at 37° C. for 50 min. Finally, the absorbance was measured at λₘₐₓ of 335 nm using a BioTek Synergy Microplate Reader (Winooski, VT).

TABLE 3
Dry powder compositions of CpG 1826 ODNs prepared using thin-film freeze-drying.

| | CpG 1826 ODNs | Sucrose | Trehalose | Mannitol | Lactose | Leucine | TE buffer slats |
|---|---|---|---|---|---|---|---|
| Composition | (% w/w) | (% w/w) | (% w/w) | (% w/w) | (% w/w) | (% w/w) | (% w/w) |
| Composition-1 | 0.4 | 41 | 0 | 0 | 0 | 27 | 31.6 |
| Composition-2 | 0.4 | 58 | 0 | 0 | 0 | 10 | 31.6 |
| Composition-3 | 0.4 | 0 | 41 | 0 | 0 | 27 | 31.6 |
| Composition-4 | 0.4 | 0 | 58 | 0 | 0 | 10 | 31.6 |
| Composition-5 | 0.4 | 0 | 0 | 41 | 0 | 27 | 31.6 |
| Composition-6 | 0.4 | 0 | 0 | 58 | 0 | 10 | 31.6 |
| Composition-7 | 0.3 | 49 | 0 | 0 | 0 | 32 | 18.7 |
| Composition-8 | 0.3 | 69 | 0 | 0 | 0 | 12 | 18.7 |
| Composition-9 | 0.3 | 0 | 49 | 0 | 0 | 32 | 18.7 |
| Composition-10 | 0.3 | 0 | 69 | 0 | 0 | 12 | 18.7 |
| Composition-11 | 0.3 | 0 | 0 | 49 | 0 | 32 | 18.7 |
| Composition-12 | 0.3 | 0 | 0 | 69 | 0 | 12 | 18.7 |
| Composition-13 | 0.4 | 0 | 0 | 0 | 58 | 10 | 31.6 |
| Composition-14 | 0.3 | 0 | 0 | 0 | 49 | 32 | 18.7 |
| Composition-15 | 1 | 0 | 0 | 0 | 48 | 32 | 19 |
| Composition-16 | 1 | 0 | 48 | 0 | 0 | 32 | 19 |

Agarose gel electrophoresis. A 2% w/v agarose gel was prepared and pre-stained with 1 μg/mL ethidium bromide. The same concentration of dye was used in the TAE running buffer (40 mM Tris-acetate, 1 mM EDTA, pH 8.3). Samples (i.e., reconstituted composition-15 and composition-16 dry powders, their liquid counterparts and original CpG 1826 ODNs in TE) were loaded in a loading dye. The electrophoresis was run for 30 min at 100 V.

Immunostimulatory capacity of representative CpG 1826 ODN dry powder compositions in J774A.1 murine macrophages. Cells (1×10⁴ cells/well) were treated TE buffer as a negative control, lipopolysaccharide (LPS, 100 ng/mL), original CpG 1826 ODNs in TE buffer, composition-15 or composition-16, before and after TFFD and reconstitution for 24 h. The CpG 1826 ODN concentration was 2 μM. The concentrations of cytokines (IL-6, IL-12 (p40) and TNF-α) in the culture supernatants were determined using ELISA kits.

Dry powder characterization. Powder crystallinity was evaluated using a XtaLAB Synergy (Rigaku Oxford Diffraction) Dual Source Diffractometer equipped with a HyPix6000E detector (Tokyo, Japan) using a microfocus sealed X-ray tube with CuKα radiation source (λ 1.5418 Å) as described before [3, 4]. The microstructures of representative dry powders (i.e., composition-15 and composition-16) were analyzed using a Hitachi S5500 scanning electron microscope with an acceleration voltage of 20 kV. A thin film was loaded onto a conductive carbon tape. The sample was then coated with a layer of Au/Pd (60:40) using a sputter coater (40 mA, 1 min) before capturing the images. The Residual moisture contents in composition-15 and composition-16 powders were determined by coulometric Karl Fischer titration using a Mettler Toledo C20 coulometer (Columbus, OH) [5]. Briefly, the powder content of a vial was dissolved in 2.5 mL of Coulomat AG solution in air-tight conditions. The solution was mixed well until the powder was dissolved, and 2.0 mL of the solution was then injected back to the solution tank. The amount of water in the samples was determined and the residual moisture content (% w/w) in the powders was calculated using the equation below:

$$Residual moisture ( \% w / w ) = \frac{Amount of water ( mg )}{Weight of powder ( mg )} \times 100$$

Results. For pulmonary delivery into the lungs, an adequate amount of aerosolized dry powders must deposit in the target regions of the lungs (Gagnadoux et al., 2008). The deposition pattern of dry powder within the human respiratory tract is mainly mediated by the powder's aerodynamic diameter (Zhang et al., 2021). The aerosol performance of CpG 1826 ODN dry powders was studied using an NGI. FIG. 17 demonstrates the aerosol properties of representative dry powder compositions. The aerodynamic diameter is usually expressed as the median mass aerodynamic diameter (MMAD) (Carvalho et al., 2011). The MMAD of the dry powder compositions ranged between 1.3 and 4.2 μm (FIG. 17). Therefore, it is expected that the powders can potentially achieve efficient deposition within the lungs following oral inhalation (AboulFotouh et al., 2020). In particular, particles with a MMAD value around 1.5 μm mainly deposit in the alveolar region (Borghardt et al., 2018). As illustrated in FIG. 17, >75% of the emitted doses of compositions 4, 9, 14, 15 and 16 had a MMAD of ≤5 μm (FPF≤5 μm). Thus, the major fraction of the emitted dose is expected to reach the deep lungs (i.e., the alveolar region). NSCLC originates from the epithelial cells of central bronchi to terminal alveoli, and therefore deep lung deposition is critical for efficient anticancer activity of primary lung cancer and pulmonary metastases. Thin-film freeze-drying commonly leads to the formation of fragile matrix powders characterized by a significantly porous microstructure, which can be fragmented into inhalable microparticles through the utilization of passive DPIs (Watts et al., 2011). Overall, data in FIG. 17 indicated that thin-film freeze-drying can be used to develop dry powders of CpG 1826 ODNs that can potentially deposit in the alveolar region upon oral inhalation using a DPI device. CpG 1826 ODN compositions 15 and 16 contained a high percentage of CpG 1826 ODNs and showed desirable aerosol properties. Therefore, they were used for additional studies.

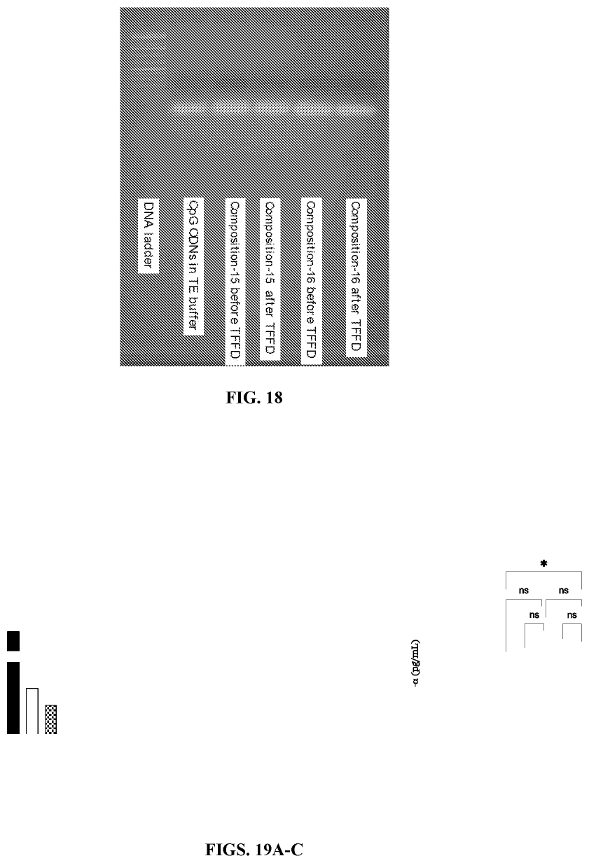

The effect of thin-film freeze-drying on the integrity of CpG 1826 ODNs was investigated using agarose gel electrophoresis. As depicted in FIG. 18, the integrity of CpG 1826 ODNs was maintained in the thin-film freeze-dried powder following reconstitution. Then the immunostimulatory capacity of the CpG 1826 ODNs following drying and reconstitution was studied in J774A.1 murine macrophages. The results showed that the CpG 1826 ODNs reconstituted from dry powder compositions 15 and 16 effectively stimulated the J774A.1 murine macrophages to produce cytokines such as IL-6, IL-12 (p40), and TNF-α, and as illustrated in FIG. 19, the concentrations of the cytokines elicited by CpG 1826 ODNs reconstituted from dry powder compositions 15 and 16 were comparable to those elicited by the corresponding liquid formulations before thin-film freeze-drying or the original CpG 1826 ODNs.

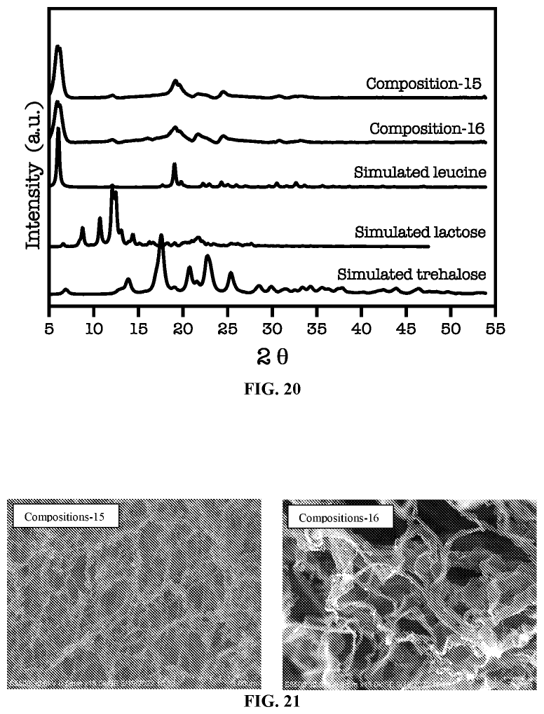

Powder X-ray diffraction (PXRD) patterns of dry powder compositions 15 and 16 comprising leucine and lactose or trehalose as a stabilizer, respectively, at a concentration of 0.48% w/w are shown in FIG. 20. Trehalose and lactose showed amorphous glassy matrices while leucine was crystalline. During drying, water molecules are replaced by the formation of hydrogen bonds between the lyoprotectant (i.e., trehalose or lactose) and the polar groups in the CpG 1826 ODNs (Clegg et al., 1982). The hydrogen bonding capability is maximized in the amorphous state since the molecules are less restrained than the ordered crystalline state (Jensen et al., 2012).

Representative scanning electron micrographs of dry powder compositions 15 and 16 prepared using lactose or trehalose as a stabilizer, respectively, are shown in FIG. 21. Both dry powder compositions were porous, though with different matrix microstructures based on the stabilizer used (lactose vs trehalose).

The moisture content in dry powder compositions 15 and 16 was about 1% w/w (Table 4). Minimizing the residual moisture content in the dry powders is crucial to ensure the stability of the powders as well as the active ingredient(s) in the powders. High residual moisture in the dry powders is associated with numerous undesirable effects including microbial growth, RNase contamination, particle aggregation, low glass transition temperatures (Tg), poor powder flow, compromised aerosol performance, and ultimately poor product quality [4, 9, 15-17]. Therefore, it is recommended to maintain the residual moisture content in the biologic powders at sufficiently low level (typically <2%) (Preston & Randolph, 2021).

TABLE 4
Residual moisture content in dry powder compositions 15
and 16. Data are mean ± S.D. (n = 3).

| Formulation | Residual moisture content (% w/w) |
|---|---|
| Composition-15 | 0.68 ± 0.44 |
| Composition-16 | 1.01 ± 0.66 |

### Example 6. Inhalable Dry Powder Compositions of Toll-Like Receptor Agonists Prepared Using the Thin-Film Freeze-Drying Technology

In this example, thin-film freeze-drying was utilized to formulate inhalable dry powder compositions for various TLR agonists namely, polyadenylic-polyuridylic acid (poly(A:U), a TLR3 agonist, InvivoGen), poly(U) (a TLR8 agonist, InvivoGen), and peptidoglycan from B. subtills (PGN-BS, a TLR2 agonist, InvivoGen). Liquid formulations of various TLR agonists comprising leucine and lactose or trehalose (Table 5) were dried using thin-film freeze-drying as described in Example 7. Subsequently, the aerosol properties of various dry powder compositions were determined using an NGI.

TABLE 5
Compositions of liquid formulations comprising TLR agonists.

| | Poly(A:U) | Poly(U) | PGN-BS | Lactose | Trehalose | Leucine |
|---|---|---|---|---|---|---|
| Composition | (% w/v) | (% w/v) | (% w/v) | (% w/v) | (% w/v) | (% w/v) |
| Composition-20 | 0.01 | 0 | 0 | 0.48 | 0 | 0.32 |
| Composition-21 | 0 | 0.001 | 0 | 0.48 | 0 | |
| Composition-22 | 0 | 0 | 0.001 | 0 | 0.48 | |

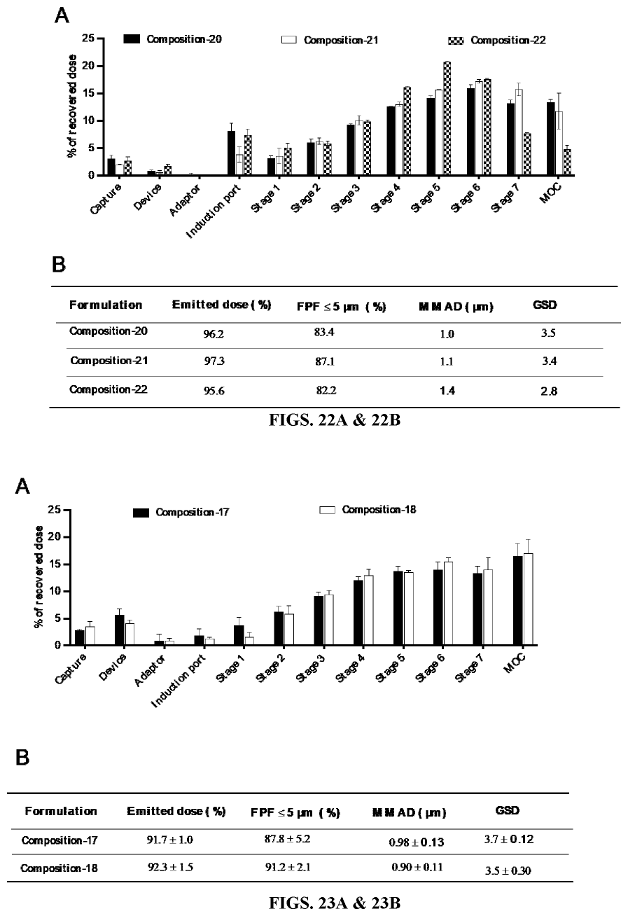

FIG. 22 demonstrates the aerosol properties of dry powder compositions comprising various TLR agonists. The MMAD of the dry powder compositions ranged between 1.0 and 1.4 μm (FIG. 22). In particular, particles with an MMAD value around 1.5 μm mainly deposit in the alveolar region (Borghardt et al., 2018). Additionally, >80% of the emitted doses had an MMAD of ≤5 μm (FPF≤5 μm). Therefore, it is expected that the TLR ligand powders can potentially achieve efficient deposition within the lungs following oral inhalation (AboulFotouh et al., 2020).

### Example 7: Inhalable Dry Powder Compositions of CpG 1826 ODN-Adjuvanted Vaccines

Vaccine delivery to different regions of the respiratory tract can elicit both local and systemic immune responses. However, antigen distribution within the respiratory tract prominently affects the elicited immune responses. It is expected that lung deposition is beneficial for eliciting protective immune responses (Tonnis et al., 2012). For instance, the extent of virus-specific immune responses elicited by a monovalent H3N2 influenza vaccine increased with the depth of antigen deposition within the respiratory tract. Deep lung deposition of the vaccine produced significantly higher mucosal specific IgA levels than when the vaccine was deposited main in the upper or central airways. It also resulted in shifting the cellular immune response towards Th-1 compared to the intramuscular injection (Minne et al., 2007).

In this example, thin-film freeze-drying was utilized to formulate inhalable dry powder compositions for CpG 1826 ODN-adjuvanted ovalbumin (OVA, Sigma-Aldrich). OVA was used as a model vaccine antigen. Liquid formulations of various vaccines in PBS (pH 7.4, 10 mM) or TE buffer comprising leucine and lactose were dried using thin-film freeze-drying as described in Example 7. Subsequently, the aerosol properties of various dry powder compositions (Table 6) were determined using NGI as described in Example 1.

TABLE 6
The compositions of the CpG 1826 ODN-adjuvanted OVA model
vaccines dry powders prepared using thin-film freeze-drying

| | CpG 1826 ODNs | Ovalbumin | Lactose | Leucine | Tris-EDTA buffer | PBS buffer |
|---|---|---|---|---|---|---|
| Composition | (% w/w) | (% w/w) | (% w/w) | (% w/w) | slats (% w/w) | salts (% w/w) |
| Composition- | 1 | 3 | 47 | 31 | 18 | 0 |
| 17 | | | | | | |
| Composition- | 0.1 | 1.9 | 26 | 18 | 0 | 54 |
| 18 | | | | | | |

FIG. 23 demonstrates the aerosol properties of dry powder compositions of CpG 1826 ODN-adjuvanted OVA model vaccines. The MMAD values of the dry powder compositions were about 1 μm (FIG. 23). Additionally, >85% of the emitted doses had a MMAD of ≤5 μm (FPF≤5 μm). Therefore, it is expected that the powders can potentially achieve efficient deposition within the lungs following oral inhalation for pulmonary immunization (AboulFotouh et al., 2020).

### Example 8: Dry Powder Compositions of Peptides, Plasmid DNA, Live-Attenuated Viruses, Bacteriophages, and Bacteria for Respiratory Delivery

Background. Respiratory delivery (e.g., intranasal and/or pulmonary delivery) is non-invasive and can be used in a range of local and systemic conditions. It provides numerous advantages including ease of administration, rapid onset of action, and bypassing the first-pass metabolism (Grassin-Delyle et al., 2012). Additionally, mucosal vaccination can potentially induce both mucosal and systemic immune responses (Xu et al., 2014). For instance, intranasal administration of spike protein-encoding DNA complexed in the cationic polymer polyethyleneimine induced antigen-specific humoral and cellular immune responses in mice (Shim et al., 2010). Similarly, pulmonary or intranasal administration of live-attenuated measles vaccine completely protected rhesus macaques from infection with wild-type measles virus for more than one year (Lin et al., 2011). In addition, respiratory delivery is effective in combating bacterial infections. For instance, pulmonary delivery of bacteriophages can directly combat bacterial infections in the lungs in particular those caused be antibiotic-resistant bacteria (Abedon, 2015).

To prepare dry powder compositions loaded with various biologics and/or nucleic acid-based products such as peptides, proteins, modified proteins (e.g., PEGylated cytokines), enzymes, protein-drug conjugates, viruses, bacteriophages, bacteria, plasmid DNA, and other nucleic acid-based products (e.g., siRNA, miRNA, short RNA, antisense oligos, RNA in extracellular vesicles) for respiratory delivery using the thin-film freeze-drying technology, the active ingredient will be mixed with various excipients before thin-film freezing and drying. Excipients include but are not limited to: (i) one or more sugars or sugar alcohol such as sucrose, trehalose, lactose, and mannitol; (ii) buffers such as Tris buffer, histidine buffer, HEPES buffer, Tris-EDTA buffer, citrate buffer, phosphate buffers, or phosphate-buffered saline; (iii) mucoadhesive agents such as carboxymethyl cellulose, chitosan, N-trimethyl chitosan chloride, chitosan glutamate, Carbopol, and hyaluronic acid; (iv) amino acids such as leucine; (v) polymers such as polyethyleneimine, and poly(lactic-co-glycolic acid) and (vi) lipids such as DSPC, DOPE and cholesterol. Liquid formulations will be frozen into thin films at optimized temperatures (i.e., between about −20 to −180° C.) and then lyophilized using an optimized lyophilization cycle.

The dry powders prepared using thin-film freeze-drying are expected to be porous with brittle matrices that can be sheared into particles with the optimal size range for either intranasal or pulmonary deposition using the appropriate intranasal delivery device or dry powder inhaler, respectively. The aerosol properties of the powder can be adjusted by adjusting the solid content of the liquid formulations before drying. For instance, dry powders with large aerodynamic particle size that are expected to deposit in the nasal cavity and the upper respiratory tract may be prepared using solid content of about 3% (w v) or above, whereas powder with small aerodynamic diameters that tend to deposit in the lungs may be prepared using solid content of less than 3% (w v). The residence time of the powders in the respiratory tract may be modulated by adjusting the amount of mucoadhesive polymer(s) in the formulation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### V. References

The following references, to the extent that they provide exemplary procedural or other details supplementary to those set forth herein, are specifically incorporated herein by reference.
U.S. Patent Application No. 2010/0221343
Abedon, Bacteriophage, 2015. 5(1): p. e1020260.
AboulFotouh et al., Int J Pharm, 587 (2020) 119711.
AboulFotouh et al., Int J Pharm, 2022. 622: p. 121825.
Barnard et al., J. Pharm. Sci., 2011. 100(2): p. 492-503.
Birkhoff et al., Indian J Pharm Sci, 2009, 71(6): p. 729-731.
Borghardt et al., Can Respir J, 2018 (2018) 2732017.
Cahn et al., Nat Rev Bioeng, 2023. 1(2): p. 83-84.
Carvalho et al., Int J Pharm, 2011 Vol. 406 Issue 1-2 Pages 1-10.
Clegg et al., Cryobiology, 1982 Vol. 19 Issue 3 Pages 306-16
Davies et al., Mol Ther Nucleic Acids, 2021. 24: p. 369-384.
Dekina et al., Carbohydr Polym, 2016. 147: p. 208-215.
Djupesland, Drug. Deliv. Transl. Res., 2013. 3(1): p. 42-62.
Emig et al., bioRxiv, 2021: p. 2021.10.12.464150.
Engstrom et al., Pharm. Res., 2008. 25(6): p. 1334-1346.
Filipović-Grčić & Hafner, Pharmaceutical Sciences Encyclopedia. p. 1-32.
Gagnadoux et al., J Aerosol Med Pulm Drug Deliv, 2008 Vol. 21 Issue 1 Pages 61-70.
Glücklich et al., Int. J. Pharm., 2020. 591: p. 119934.
Grabovac et al., Adv Drug Deliv Rev, 2005. 57(11): p. 1713-1723.
Grassin-Delyle et al., Pharmacol Ther, 2012. 134(3): p. 366-79.
Haeuser et al., J. Pharm. Sci., 2020. 109(1): p. 807-817.
Halwe et al., Viruses, 2021. 13(8): p. 1498.
Hassett et al., J Control Release, 2021. 335: p. 237-246.
Heo, Drugs, 2022. 82(4): p. 477-484.
Hufnagel et al., Int. J. Pharm., 2022. 618: p. 121637.
Igyarto et al., Curr Opin Virol, 2021. 48: p. 65-72.
Jain et al., StatPearls. 2022, StatPearls Publishing: Treasure Island (FL).
Jensen et al., J Control Release, 2012 Vol. 157 Issue 1 Pages 141-8.
Kalonia et al., J. Pharm. Sci., 2015. 104(2): p. 536-547.
Keam, Drugs, 2022. 82(9): p. 1001-1010.
Kelley et al., Curr. Opin. Biotechnol., 2022. 78: p. 102798.
Kesavan et al., Sci Pharm, 2010. 78(4): p. 941-957.
Kim & Jang, CEVR, 2017. 6(1): p. 15-21.
Li et al., Int. J. Pharm., 2000. 199(1): p. 65-76.
Lin et al., Proc Natl Acad Sci USA, 2011. 108(7): p. 2987-92.
Mohammed et al., Eur. J. Pharm. Sci., 2007. 30(5): p. 406-413.
Minne et al., Immunology, 122 (2007) 316-325.
Moreira et al., Front. Immunol., 2021. 12: p. 709861.
Nižić Nodilo et al., Pharmaceutics, 2021. 13(6): p. 795.
Okuda et al., J Control Release, 2022. 348: p. 648-659.
Perry et al., ACS Nano, 2020. 14(6): p. 7200-7215.
Petersen et al., Lancet Infect. Dis., 2020. 20(9): p. e238-e244.
Pormohammad et al., Rev. Med. Virol., 2021. 31(3): p. e2179.
Praphawatvet et al., Int. J Pharm., 2022. 629: p. 122357.
Preston & Randolph, Adv Drug Deliv Rev, 2021 Vol. 171 Pages 50-6.
Sahakijpijarn et al., Pharmaceutics, 2020. 12(11).
Sasaki, 2022. 14(8).
Schoenmaker et al., Int J Pharm, 2021. 601: p. 120586.
Shim et al., BMC Immunol, 2010. 11: p. 65.
Sogias et al., Biomacromolecules, 2008. 9(7): p. 1837-1842.
Tonnis et al., J Aerosol Med Pulm Drug Deliv, 25 (2012) 249-260.
Trenkel & Scherließ, Pharmaceutics, 2021. 13(3): p. 385.
van Riel et al., Am. J. Clin. Pathol., 2010. 176(4): p. 1614-1618.
Wang et al., Int. J Pharm., 2023. 630: p. 122408.
Warnken et al., Mol Pharmaceutics, 2018. 15(4): p. 1392-1402.
Watts et al., Pharm. Res. 2013, 30 (3), 813-25.
Weng et al., Biotechnol Adv, 2020. 40: p. 107534.
Whitehead et al., Nat Commun, 2014. 5: p. 4277.
Xu et al., Pharmaceutics, 2014. 6(3): p. 378-415.
Xu et al., Int J Pharm, 2021. 609: p. 121126.
Zhang et al., Adv Drug Deliv Rev, 2021 Vol. 172 Pages 183-210.
Zhang & Gao, Mater Sci Eng C Mater Biol Appl, 2017. 70(Pt 2): p. 935-946.

1. A pharmaceutical composition comprising:
(A) a therapeutic composition comprising:
(i) an active pharmaceutical ingredient;
(ii) an excipient, wherein the excipient is selected from a sugar, sugar alcohol, or an amino acid;
(iii) a mucoadhesive excipient; and
(B) a nasal delivery device, wherein the therapeutic composition is loaded in the nasal device or formulated into a container that can be attached to the nasal delivery device; and the nasal delivery device produces an initial velocity upon delivering the therapeutic composition of at least 200 cm/s.

2. (canceled)

3. (canceled)

4. The pharmaceutical composition of claim 1, wherein the active pharmaceutical ingredient further comprises an adjuvant.

5.-11. (canceled)

12. The pharmaceutical composition of claim 1, wherein the active pharmaceutical ingredient is an antigen.

13.-21. (canceled)

22. The pharmaceutical composition of claim 1, wherein the active pharmaceutical ingredient is formulated into or admixed a liposome with at least one lipid and one sterol.

23.-29. (canceled)

30. The pharmaceutical composition of claim 1, wherein the mucoadhesive excipient is a charged cellulosic polymer.

31. (canceled)

32. (canceled)

33. The pharmaceutical composition of claim 1, wherein the mucoadhesive excipient comprises an amount from about 0.1% to about 16% w/w of the composition relative to the total weight of the components.

34.-44. (canceled)

45. The pharmaceutical composition of claim 1, wherein the pharmaceutical composition further comprises a further excipient.

46. The pharmaceutical composition of claim 45, wherein the further excipient is a buffer.

47.-64. (canceled)

65. A method of preferentially delivering an active pharmaceutical ingredient to either the lower turbinate, middle turbinate, or the nasopharynx region of the nasal cavity comprising administering to the nasal cavity of the person a pharmaceutical composition of claim 1, wherein at least 45% of the active pharmaceutical ingredient is delivered to the lower turbinate, middle turbinate, or the nasopharynx region.

66.-83. (canceled)

84. A method of delivering a biologic active agent to the nasal cavity of a patient comprising:
(A) obtaining a pharmaceutical composition comprising:
(1) a biologic active agent;
(II) a sugar or sugar alcohol; and
(III) a buffer;
wherein the pharmaceutical composition comprises a plurality of drug particles having an average diameter from about 50 μm to about 1 cm;
(B) delivering the biologic active agent to the nasal cavity by ejecting the powder from a device into the nostril.

85. The method of claim 84, wherein the biologic active agent is an antibody or a fragment of an antibody.

86.-101. (canceled)

102. The method of claim 84, wherein the pharmaceutical composition further comprises one or more excipients.

103.-114. (canceled)

115. The method of claim 84, wherein the pharmaceutical composition comprises:
(A) a biologic active agent, wherein the biologic active agent is an antibody or antibody fragment;
(B) a sugar or sugar alcohol; wherein the ratio of the sugar or sugar alcohol to the biologic active agent is greater than 5:1;
(C) a buffer, wherein the buffer has a pH range is from about pH 6 to 8;
(D) an excipient, wherein the excipient is an amino acid; and
(E) a surfactant, wherein the surfactant is a non-ionic surfactant.

116.-127. (canceled)

128. The method of claim 84, wherein the pharmaceutical composition further comprises an excipient.

129. The method of claim 128, wherein the excipient is an amino acid.

130.-134. (canceled)

135. The method of claim 84, wherein the buffer further comprises a chelating compound.

136.-176. (canceled)

177. A method of preparing a pharmaceutical composition comprising:
(A) dissolving a precursor solution in a solvent to obtain a pharmaceutical mixture; wherein the precursor solution comprises:
(1) a biologic active agent;
(II) a sugar or sugar alcohol; and
(III) a buffer;
(B) applying the pharmaceutical mixture to a surface at a surface temperature below 0° C. to obtain a frozen pharmaceutical mixture; wherein the pharmaceutical mixture comprises at least about 3% by weight of the precursor solution; and
(C) collecting the frozen pharmaceutical mixture and drying the frozen pharmaceutical mixture to obtain a pharmaceutical composition.

178.-180. (canceled)

181. The method of claim 177, wherein the pharmaceutical mixture comprises a solid content from about 3% w/v to about 15% w/v of the precursor solution.

182.-195. (canceled)

196. The method of claim 177, wherein the frozen pharmaceutical composition is dried by lyophilization.

197.-271. (canceled)

272. The pharmaceutical composition of claim 1, wherein the active pharmaceutical ingredient is a nucleic acid in an extracellular vesicle.
